(19) United States
(12) Patent Application Publication
(10) Pub. No.: US 2007/0185107 A1
(43) Pub. Date: Aug. 9, 2007

(54) Chroman compounds

(75) Inventors: Peter Bernstein, Wilmington, DE (US); Daniel Hill, Wilmington, DE (US); David Nugiel, Wilmington, DE (US); Edward Pierson, Wilmington, DE (US); Ashokkumar Shenvi, Wilmington, DE (US); Robert Jacobs, Durham, NC (US)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: AstraZeneca AB, Sodertalje (SE)

(21) Appl. No.: 11/592,686

(22) Filed: Nov. 3, 2006

Related U.S. Application Data
(60) Provisional application No. 60/733,993, filed on Nov. 4, 2005.

(51) Int. Cl. A61K 31/5377; A61K 31/497; C07D 413/14; C07D 405/14

(52) U.S. Cl. 514/235.5; 514/252.11; 544/121; 544/357

(57) ABSTRACT

Compounds according to Formula (I): 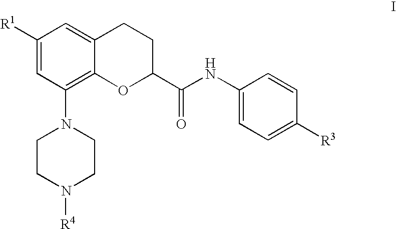
wherein R¹, R², R³, and R⁴ are as defined in the specification, pharmaceutically-acceptable salts, methods of making, pharmaceutical compositions containing and methods for using the same.

### FIELD OF THE INVENTION

This invention relates to novel chroman derivatives, methods for their preparation, pharmaceutical compositions containing them and their use in therapy.

### BACKGROUND OF THE INVENTION

Alterations in serotonin (5-hydroxytryptamine or 5-HT) activity has been implicated in many psychiatric disorders including but not limited to depression, generalized anxiety, eating disorders, dementia, panic disorder, and sleep disorders. Furthermore serotonin has been implicated in gastrointestinal disorders, cardiovascular regulation, motor disorders, endocrine disorders, vasospasm and sexual dysfunction. Serotonin receptors have been subdivided into at least 14 subtypes, see Barnes and Sharp, Neuropharmacology, 1999, 38, 1083-1152. These various subtypes are responsible for serotonin's action in many pathophysiological conditions. The 5-HT₁ families of receptors have high affinity for serotonin and consist of five related receptors. This family includes the 5-HT_{1B} and 5-HT_{1D} receptor subtypes. Compounds that interact with the 5-HT₁ families are known to have therapeutic potential in the above-mentioned disorders and diseases. In particular, compounds that are 5-HT_{1B} and 5-HT_{1D} antagonist have been known to be antidepressant and anxiolytic agents. The present invention discloses several 5-HT_{1B} antagonists that are useful for the treatment of anxiety disorders or mood disorders such as depression or dementia and other cognitive disorders such as Alzheimer's disease.

Provided herein are compounds in accord with Formula I: 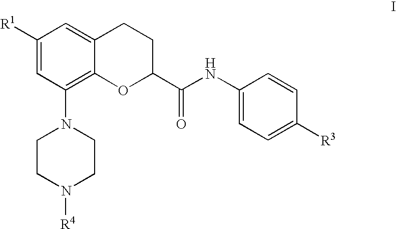
wherein:
R¹ is F or OCH₃;
R⁴ is H, CH₃, C₂₋₄alkyl, CH₂CH₂OCH₃, or CH₂CF₃; and
R³ is selected from morpholino or piperazine attached by nitrogen and optionally substituted on the other nitrogen (for piperazine) with —C(═O)CH₃, —CH₃, —C(═O)CH₂CH₃, —C(═O)N(CH₃)₂, —SO₂CH3, or —SO₂CH₂CH₃;
with the proviso that when R¹ is F and R⁴ is methyl then R³ cannot be morpholine; and
with the proviso that when R¹ is F and R⁴ is methyl then R³ cannot be piperazine substituted with —SO₂CH3, or —SO₂CH₂CH₃;

or a pharmaceutically-acceptable salt thereof.

Also provided are methods of using the compounds of formula I to treat diseases and conditions associated with a wide range of diseases or disorders in which 5-HT receptors are considered to have a role. Also provided are uses of the compounds of formula I as medicaments, uses of the compounds of formula I in the manufacture of medicaments and uses of the compounds of formula I for diagnostic and analytic purposes. Also provided are various administration methods either alone or in combination with other therapeutically active compounds or substances. Also provided are processes and intermediates used to prepare the compounds of formula I. Also provided are pharmaceutical compositions containing the compounds of formula I.

### DEFINITIONS

If Used Herein, the Following Terms have the Following Meanings:

The term “amine” or “amino” refers to radicals of the general formula —NRR′, wherein R and R′ are independently selected from hydrogen or a hydrocarbyl radical.

The term “hydrocarbyl” refers to any structure comprising only carbon and hydrogen atoms up to 14 carbon atoms.

The term “alkyl” used alone or as a suffix or prefix, refers to straight or branched chain hydrocarbyl radicals comprising 1 to about 12 carbon atoms.

The term “aromatic” refers to hydrocarbyl radicals having one or more polyunsaturated carbon rings having aromatic character, (e.g., 4n+2 delocalized electrons) and comprising 6 up to about 14 carbon atoms.

The term “aryl” refers to aromatic radicals including both monocyclic aromatic radicals comprising 6 carbon atoms and polycyclic aromatic radicals comprising up to about 14 carbon atoms.

The term “halo” or “halogen” refers to fluorine, chlorine, bromine and iodine radicals.

The term “heterocycle” or “heterocyclic” or “heterocyclic moiety” refers to ring-containing monovalent and divalent radicals having one or more heteroatoms, independently selected from N, O and S, as part of the ring structure and comprising at least 3 and up to about 20 atoms in the rings preferably 5 and 6 membered rings. Heterocyclic moieties may be saturated or unsaturated, containing one or more double bonds, and heterocyclic moieties may contain more than one ring.

The term “heteroaryl” refers to heterocyclic monovalent and divalent radicals having aromatic character.

Heterocyclic moieties include for Example monocyclic moieties such as: aziridine, oxirane, thiirane, azetidine, oxetane, thietane, pyrrolidine, pyrroline, imidazolidine, pyrazolidine, dioxolane, sulfolane 2,3-dihydrofuran, 2,5-dihydrofuran tetrahydrofuran, thiophane, piperidine, 1,2,3,6-tetrahydro-pyridine, piperazine, morpholine, thiomorpholine, pyran, thiopyran, 2,3-dihydropyran, tetrahydropyran, 1,4-dihydropyridine, 1,4-dioxane, 1,3-dioxane, dioxane, homopiperidine, 2,3,4,7-tetrahydro-1H-azepine homopiperazine, 1,3-dioxepane, 4,7-dihydro-1,3-dioxepin, and hexamethylene oxide. In addition heterocyclic moieties include heteroaryl rings such as: pyridyl, pyrazinyl, pyrimidinyl, pyridazinyl, thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4 oxadiazolyl. Additionally, heterocyclic moieties encompass polycyclic moieties such as: indole, indoline, quinoline, tetrahydroquinoline, isoquinoline, tetrahydroisoquinoline, 1,4-benzodioxan, coumarin, dihydrocoumarin, benzofuran, 2,3-dihydrobenzofuran, 1,2-benzisoxazole, benzothiophene, benzoxazole, benzthiazole, benzimidazole, benztriazole, thioxanthine, carbazole, carboline, acridine, pyrolizidine, and quinolizidine.

In addition to the polycyclic heterocycles described above, heterocyclic moieties include polycyclic heterocyclic moieties wherein the ring fusion between two or more rings comprises more than one bond common to both rings and more than two atoms common to both rings. Examples of such bridged heterocycles include quinuclidine, diazabicyclo[2.2.1]heptane and 7-oxabicyclo[2.2.1]heptane.

The term “anxiety disorders” includes but is not limited to one or more of the following, panic disorder, panic disorder without agoraphobia, panic disorder with agoraphobia, agoraphobia without history of panic disorder, specific phobia, social phobia, social anxiety disorder, obsessive-compulsive disorder, posttraumatic stress disorder, acute stress disorder, generalized anxiety disorder and generalized anxiety disorder due to a general medical condition.

The term “cognitive disorders” includes but is not limited to Alzheimer's disease, dementia, and dementia due to Alzheimer's disease, dementia due to Parkinson's disease.

The term “mammal” includes all air-breathing animals including humans.

The term “mood disorders” includes but is not limited to one or more of the following, depressive disorders, including but not limited to major depressive disorder and dysthymic disorder and b) bipolar depression and/or bipolar mania including but not limited to bipolar I, including but not limited to those with manic, depressive or mixed episodes, and bipolar II, c) cyclothymic disorder, mood disorder due to a general medical condition and manic episodes associated with bipolar disorder and mixed episodes associated with bipolar disorder.

The above conditions and disorder are defined for example in the American Psychiatric Association: Diagnostic and Statistical Manual of Mental Disorders, Fourth Edition, Text Revision, Washington, D.C., American Psychiatric Association, 2000.

### DETAILED DESCRIPTION OF THE INVENTION

A further aspect of the invention provides a compound according to Formula I wherein R¹ is F; or a pharmaceutically-acceptable salt thereof.

A further aspect of the invention provides a compound according to Formula I wherein R⁴ is CH₃, or C₂₋₄alkyl; or a pharmaceutically-acceptable salt thereof.

A further aspect of the invention provides a compound according to Formula I wherein R¹ is F and R⁴ is CH₃, —CH₂CH₃, —CH₂CH₂CH₃, or —CH(CH₃)₂; or a pharmaceutically-acceptable salt thereof.

A further aspect of the invention provides a compound according to Formula I wherein R¹ is F and R⁴ is —CH₂CH₃, —CH₂CH₂CH₃, or —CH(CH₃)₂; or a pharmaceutically-acceptable salt thereof.

A further aspect of the invention provides a compound according to Formula I wherein R¹ is F and R⁴ is CH₃; or a pharmaceutically-acceptable salt thereof.

A further aspect of the invention provides a compound according to Formula I wherein R¹ is F and R³ is morpholine, with the proviso that R⁴ is not methyl; or a pharmaceutically-acceptable salt thereof.

A further aspect of the invention provides a compound according to Formula I wherein R¹ is F and R³ is piperazine attached by nitrogen and optionally substituted on the other nitrogen with —C(═O)CH₃, —CH₃, —C(═O)CH₂CH₃, —C(═O)N(CH₃)₂, —SO₂CH₂CH₃, or —SO₂CH₃, or a pharmaceutically-acceptable salt thereof.

A further aspect of the invention provides a compound according to Formula I wherein R¹ is F and R³ is piperazine attached by nitrogen and optionally substituted on the other nitrogen with —C(═O)CH₃, —CH₃, —C(═O)CH₂CH₃, or —C(═O)N(CH₃)₂ or a pharmaceutically-acceptable salt thereof.

A further aspect of the invention provides a compound according to Formula I wherein R¹ is F and R³ is piperazine attached by nitrogen and optionally substituted on the other nitrogen with —C(═O)CH₃, or —C(═O)CH₂CH₃ or a pharmaceutically-acceptable salt thereof.

A further aspect of the invention provides a compound according to Formula I wherein R¹ is F and R³ is piperazine attached by nitrogen and optionally substituted on the other nitrogen with —C(═O)CH₃ or a pharmaceutically-acceptable salt thereof.

A further aspect of the invention provides a compound according to Formula I wherein R¹ is OCH₃; or a pharmaceutically-acceptable salt thereof.

A further aspect of the invention provides a compound according to Formula I wherein R¹ is OCH₃ and R⁴ is CH₃, or C₂₋₄alkyl;

or a pharmaceutically-acceptable salt thereof.

A further aspect of the invention provides a compound according to Formula I wherein R¹ is OCH₃ and R⁴ is CH₃, —CH₂CH₃, —CH₂CH₂CH₃, or —CH(CH₃)₂; or a pharmaceutically-acceptable salt thereof.

A further aspect of the invention provides a compound according to Formula I wherein R¹ is OCH₃ and R⁴ is —CH₂CH₃, —CH₂CH₂CH₃, or —CH(CH₃)₂ or a pharmaceutically-acceptable salt thereof.

A further aspect of the invention provides a compound according to Formula I wherein R¹ is OCH₃ and R⁴ is CH₃ or a pharmaceutically-acceptable salt thereof.

A further aspect of the invention provides a compound according to Formula I wherein R¹ is OCH₃ and R³ is morpholine, or a pharmaceutically-acceptable salt thereof.

A further aspect of the invention provides a compound according to Formula I wherein R¹ is OCH₃ and R³ is piperazine attached by nitrogen and optionally substituted on the other nitrogen with —C(═O)CH₃, —CH₃, —C(═O)CH₂CH₃, —C(═O)N(CH₃)₂, —SO₂CH₂CH₃, or —SO₂CH₃, or a pharmaceutically-acceptable salt thereof.

A further aspect of the invention provides a compound according to Formula I wherein R¹ is OCH₃ and R³ is piperazine attached by nitrogen and optionally substituted on the other nitrogen with —C(═O)CH₃, —CH₃, —C(═O)CH₂CH₃, or —C(═O)N(CH₃)₂ or a pharmaceutically-acceptable salt thereof.

A further aspect of the invention provides a compound according to Formula I wherein R¹ is OCH₃ and R³ is piperazine attached by nitrogen and optionally substituted on the other nitrogen with —C(═O)CH₃, or —C(═O)CH₂CH₃ or a pharmaceutically-acceptable salt thereof.

A further aspect of the invention provides a compound according to Formula I wherein R¹ is OCH₃ and R³ is piperazine attached by nitrogen and optionally substituted on the other nitrogen with —C(═O)CH3 or a pharmaceutically-acceptable salt thereof.

A further aspect of the invention provides a compound according to Formula I wherein: R¹ is F; and R⁴ is CH₃, or —CH₂CH₃; and R³ is piperazine attached by nitrogen and optionally substituted on the other nitrogen with —C(═O)CH₃ or a pharmaceutically-acceptable salt thereof.

A further aspect of the invention provides a compound according to Formula I wherein: R¹ is OCH₃; R⁴ is CH₃, or —CH₂CH₃; and R³ is morpholine; or a pharmaceutically-acceptable salt thereof.

A further aspect of the invention provides a compound selected from:
6-methoxy-8-(4-methylpiperazin-1-yl)-N-(4-morpholinophenyl)chroman-2-carboxamide;
(+)-6-methoxy-8-(4-methylpiperazin-1-yl)-N-(4-morpholinophenyl)chroman-2-carboxamide;
(−)-6-methoxy-8-(4-methylpiperazin-1-yl)-N-(4-morpholinophenyl)chroman-2-carboxamide;
N-(4-(4-acetylpiperazin-1-yl)phenyl)-6-fluoro-8-(4-methylpiperazin-1-yl)chroman-2-carboxamide;
(+)-N-(4-(4-acetylpiperazin-1-yl)phenyl)-6-fluoro-8-(4-methylpiperazin-1-yl)chroman-2-carboxamide;
(−)-N-(4-(4-acetylpiperazin-1-yl)phenyl)-6-fluoro-8-(4-methylpiperazin-1-yl)chroman-2-carboxamide;
N-(4-(4-ethanoylpiperazin-1-yl)phenyl)-6-fluoro-8-(4-methylpiperazin-1-yl)chroman-2-carboxamide;
(+)-N-(4-(4-ethanoylpiperazin-1-yl)phenyl)-6-fluoro-8-(4-methylpiperazin-1-yl)chroman-2-carboxamide;
(−)-N-(4-(4-ethanoylpiperazin-1-yl)phenyl)-6-fluoro-8-(4-methylpiperazin-1-yl)chroman-2-carboxamide;
6-fluoro-8-(4-methylpiperazin-1-yl)-N-(4-(4-propanoylpiperazin-1-yl)phenyl)chroman-2-carboxamide;
(+)-6-fluoro-8-(4-methylpiperazin-1-yl)-N-(4-(4-propanoylpiperazin-1-yl)phenyl)chroman-2-carboxamide;
(−)-6-fluoro-8-(4-methylpiperazin-1-yl)-N-(4-(4-propanoylpiperazin-1-yl)phenyl)chroman-2-carboxamide;
6-methoxy-8-(4-methylpiperazin-1-yl)-N-(4-(4-methylpiperazin-1-yl)phenyl)chroman-2-carboxamide;
(+)-6-methoxy-8-(4-methylpiperazin-1-yl)-N-(4-(4-methylpiperazin-1-yl)phenyl)chroman-2-carboxamide;
(−)-6-methoxy-8-(4-methylpiperazin-1-yl)-N-(4-(4-methylpiperazin-1-yl)phenyl)chroman-2-carboxamide;
6-fluoro-8-(4-methylpiperazin-1-yl)-N-(4-(4-methylpiperazin-1-yl)phenyl)chroman-2-carboxamide;
(+)-6-fluoro-8-(4-methylpiperazin-1-yl)-N-(4-(4-methylpiperazin-1-yl)phenyl)chroman-2-carboxamide;
(−)-6-fluoro-8-(4-methylpiperazin-1-yl)-N-(4-(4-methylpiperazin-1-yl)phenyl)chroman-2-carboxamide;
6-fluoro-8-(4-methylpiperazin-1-yl)-N-(4-(4-propanoylpiperazin-1-yl)phenyl)chroman-2-carboxamide;
(+)-6-fluoro-8-(4-methylpiperazin-1-yl)-N-(4-(4-propanoylpiperazin-1-yl)phenyl)chroman-2-carboxamide;
(−)-6-fluoro-8-(4-methylpiperazin-1-yl)-N-(4-(4-propanoylpiperazin-1-yl)phenyl)chroman-2-carboxamide;
N-(4-(4-(ethylsulfonyl)piperazin-1-yl)phenyl)-6-methoxy-8-(4-methylpiperazin-1-yl)chroman-2-carboxamide;
(+)-N-(4-(4-(ethylsulfonyl)piperazin-1-yl)phenyl)-6-methoxy-8-(4-methylpiperazin-1-yl)chroman-2-carboxamide;
(−)-N-(4-(4-(ethylsulfonyl)piperazin-1-yl)phenyl)-6-methoxy-8-(4-methylpiperazin-1-yl)chroman-2-carboxamide;
6-methoxy-N-(4-morpholinophenyl)-8-(4-(2,2,2-trifluoroethyl)piperazin-1-yl)chroman-2-carboxamide;
(+)-6-methoxy-N-(4-morpholinophenyl)-8-(4-(2,2,2-trifluoroethyl)piperazin-1-yl)chroman-2-carboxamide;
(−)-6-methoxy-N-(4-morpholinophenyl)-8-(4-(2,2,2-trifluoroethyl)piperazin-1-yl)chroman-2-carboxamide;
6-methoxy-8-(4-(2-methoxyethyl)piperazin-1-yl)-N-(4-morpholinophenyl)chroman-2-carboxamide;
(+)-6-methoxy-8-(4-(2-methoxyethyl)piperazin-1-yl)-N-(4-morpholinophenyl)chroman-2-carboxamide;
(−)-6-methoxy-8-(4-(2-methoxyethyl)piperazin-1-yl)-N-(4-morpholinophenyl)chroman-2-carboxamide;
8-(4-butylpiperazin-1-yl)-6-methoxy-N-(4-morpholinophenyl)chroman-2-carboxamide;
(+)-8-(4-butylpiperazin-1-yl)-6-methoxy-N-(4-morpholinophenyl)chroman-2-carboxamide;
(−)-8-(4-butylpiperazin-1-yl)-6-methoxy-N-(4-morpholinophenyl)chroman-2-carboxamide;
6-methoxy-N-(4-morpholinophenyl)-8-(4-propylpiperazin-1-yl)chroman-2-carboxamide;
(+)-6-methoxy-N-(4-morpholinophenyl)-8-(4-propylpiperazin-1-yl)chroman-2-carboxamide;
(−)-6-methoxy-N-(4-morpholinophenyl)-8-(4-propylpiperazin-1-yl)chroman-2-carboxamide;
8-(4-isopropylpiperazin-1-yl)-6-methoxy-N-(4-morpholinophenyl)chroman-2-carboxamide;
(+)-8-(4-isopropylpiperazin-1-yl)-6-methoxy-N-(4-morpholinophenyl)chroman-2-carboxamide;
(−)-8-(4-isopropylpiperazin-1-yl)-6-methoxy-N-(4-morpholinophenyl)chroman-2-carboxamide;
8-(4-ethylpiperazin-1-yl)-6-methoxy-N-(4-morpholinophenyl)chroman-2-carboxamide;
(+)-8-(4-ethylpiperazin-1-yl)-6-methoxy-N-(4-morpholinophenyl)chroman-2-carboxamide;
(−)-8-(4-ethylpiperazin-1-yl)-6-methoxy-N-(4-morpholinophenyl)chroman-2-carboxamide;
6-methoxy-N-(4-morpholinophenyl)-8-(piperazin-1-yl)chroman-2-carboxamide;
(+)-6-methoxy-N-(4-morpholinophenyl)-8-(piperazin-1-yl)chroman-2-carboxamide;
(−)-6-methoxy-N-(4-morpholinophenyl)-8-(piperazin-1-yl)chroman-2-carboxamide;
6-methoxy-8-(4-methylpiperazin-1-yl)-N-(4-(4-propanoylpiperazin-1-yl)phenyl)chroman-2-carboxamide;
(+)-6-methoxy-8-(4-methylpiperazin-1-yl)-N-(4-(4-propanoylpiperazin-1-yl)phenyl)chroman-2-carboxamide;
(−)-6-methoxy-8-(4-methylpiperazin-1-yl)-N-(4-(4-propanoylpiperazin-1-yl)phenyl)chroman-2-carboxamide;
4-(4-(6-methoxy-8-(4-methylpiperazin-1-yl)chroman-2-carboxamido)phenyl)-N,N-dimethylpiperazine-1-carboxamide;
(+)-4-(4-(6-methoxy-8-(4-methylpiperazin-1-yl)chroman-2-carboxamido)phenyl)-N,N-dimethylpiperazine-1-carboxamide;
(−)-4-(4-(6-methoxy-8-(4-methylpiperazin-1-yl)chroman-2-carboxamido)phenyl)-N,N-dimethylpiperazine-1-carboxamide;
4-(4-(6-methoxy-8-(4-methylpiperazin-1-yl)chroman-2-carboxamido)phenyl)-N,N-dimethylpiperazine-1-carboxamide;
(+)-4-(4-(6-methoxy-8-(4-methylpiperazin-1-yl)chroman-2-carboxamido)phenyl)-N,N-dimethylpiperazine-1-carboxamide;
(−)-4-(4-(6-methoxy-8-(4-methylpiperazin-1-yl)chroman-2-carboxamido)phenyl)-N,N-dimethylpiperazine-1-carboxamide;
4-(4-(6-fluoro-8-(4-methylpiperazin-1-yl)chroman-2-carboxamido)phenyl)-N,N-dimethylpiperazine-1-carboxamide;
(+)-4-(4-(6-fluoro-8-(4-methylpiperazin-1-yl)chroman-2-carboxamido)phenyl)-N,N-dimethylpiperazine-1-carboxamide;
(−)-4-(4-(6-fluoro-8-(4-methylpiperazin-1-yl)chroman-2-carboxamido)phenyl)-N,N-dimethylpiperazine-1-carboxamide;
6-methoxy-8-(4-methylpiperazin-1-yl)-N-(4-(4-(methylsulfonyl)piperazin-1-yl)phenyl)chroman-2-carboxamide;
(+)-6-methoxy-8-(4-methylpiperazin-1-yl)-N-(4-(4-(methylsulfonyl)piperazin-1-yl)phenyl)chroman-2-carboxamide;
(−)-6-methoxy-8-(4-methylpiperazin-1-yl)-N-(4-(4-(methylsulfonyl)piperazin-1-yl)phenyl)chroman-2-carboxamide;
N-(4-(4-ethanoylpiperazin-1-yl)phenyl)-6-methoxy-8-(4-methylpiperazin-1-yl)chroman-2-carboxamide;
(+)-N-(4-(4-ethanoylpiperazin-1-yl)phenyl)-6-methoxy-8-(4-methylpiperazin-1-yl)chroman-2-carboxamide;
(−)-N-(4-(4-ethanoylpiperazin-1-yl)phenyl)-6-methoxy-8-(4-methylpiperazin-1-yl)chroman-2-carboxamide;
6-methoxy-8-(4-methylpiperazin-1-yl)-N-(4-(4-propanoylpiperazin-1-yl)phenyl)chroman-2-carboxamide;
(+) 6-methoxy-8-(4-methylpiperazin-1-yl)-N-(4-(4-propanoylpiperazin-1-yl)phenyl)chroman-2-carboxamide;
(−)-6-methoxy-8-(4-methylpiperazin-1-yl)-N-(4-(4-propanoylpiperazin-1-yl)phenyl)chroman-2-carboxamide;
N-(4-(4-ethanoylpiperazin-1-yl)phenyl)-6-methoxy-8-(4-methylpiperazin-1-yl)chroman-2-carboxamide;
(+)-N-(4-(4-ethanoylpiperazin-1-yl)phenyl)-6-methoxy-8-(4-methylpiperazin-1-yl)chroman-2-carboxamide; and
(−)-N-(4-(4-ethanoylpiperazin-1-yl)phenyl)-6-methoxy-8-(4-methylpiperazin-1-yl)chroman-2-carboxamide; or a pharmaceutically-acceptable salt thereof.

A further aspect of the invention provides a compound according to Formula I wherein said compound is dextrorotatory, or a pharmaceutically-acceptable salt thereof.

A further aspect of the invention provides a compound selected from:
(+)-6-methoxy-8-(4-methylpiperazin-1-yl)-N-(4-morpholinophenyl)chroman-2-carboxamide;
(+)-N-(4-(4-ethanoylpiperazin-1-yl)phenyl)-6-fluoro-8-(4-methylpiperazin-1-yl)chroman-2-carboxamide;
(+)-N-(4-(4-ethanoylpiperazin-1-yl)phenyl)-6-fluoro-8-(4-methylpiperazin-1-yl)chroman-2-carboxamide;
(+)-6-fluoro-8-(4-methylpiperazin-1-yl)-N-(4-(4-propanoylpiperazin-1-yl)phenyl)chroman-2-carboxamide;
(+)-6-methoxy-8-(4-methylpiperazin-1-yl)-N-(4-(4-methylpiperazin-1-yl)phenyl)chroman-2-carboxamide;
(+)-6-fluoro-8-(4-methylpiperazin-1-yl)-N-(4-(4-methylpiperazin-1-yl)phenyl)chroman-2-carboxamide;
(+)-6-fluoro-8-(4-methylpiperazin-1-yl)-N-(4-(4-propanoylpiperazin-1-yl)phenyl)chroman-2-carboxamide;
(+)-N-(4-(4-(ethylsulfonyl)piperazin-1-yl)phenyl)-6-methoxy-8-(4-methylpiperazin-1-yl)chroman-2-carboxamide
(+)-8-(4-butylpiperazin-1-yl)-6-methoxy-N-(4-morpholinophenyl)chroman-2-carboxamide; (+)-6-methoxy-N-(4-morpholinophenyl)-8-(4-propylpiperazin-1-yl)chroman-2-carboxamide;
(+)-8-(4-isopropylpiperazin-1-yl)-6-methoxy-N-(4-morpholinophenyl)chroman-2-carboxamide;
(+)-8-(4-ethylpiperazin-1-yl)-6-methoxy-N-(4-morpholinophenyl)chroman-2-carboxamide;
(+)-6-methoxy-8-(4-methylpiperazin-1-yl)-N-(4-(4-propanoylpiperazin-1-yl)phenyl)chroman-2-carboxamide;
(+)-4-(4-(6-methoxy-8-(4-methylpiperazin-1-yl)chroman-2-carboxamido)phenyl)-N,N-dimethylpiperazine-1-carboxamide;
(+)-4-(4-(6-methoxy-8-(4-methylpiperazin-1-yl)chroman-2-carboxamido)phenyl)-N,N-dimethylpiperazine-1-carboxamide;
(+)-4-(4-(6-fluoro-8-(4-methylpiperazin-1-yl)chroman-2-carboxamido)phenyl)-N,N-dimethylpiperazine-1-carboxamide;
(+)-6-methoxy-8-(4-methylpiperazin-1-yl)-N-(4-(4-(methylsulfonyl)piperazin-1-yl)phenyl)chroman-2-carboxamide;
(+)-N-(4-(4-ethanoylpiperazin-1-yl)phenyl)-6-methoxy-8-(4-methylpiperazin-1-yl)chroman-2-carboxamide;
(+) 6-methoxy-8-(4-methylpiperazin-1-yl)-N-(4-(4-propanoylpiperazin-1-yl)phenyl)chroman-2-carboxamide; and
(+)-N-(4-(4-ethanoylpiperazin-1-yl)phenyl)-6-methoxy-8-(4-methylpiperazin-1-yl)chroman-2-carboxamide; or a pharmaceutically-acceptable salt thereof.

A further aspect of the invention provides a compound according to Formula I for use in the treatment of anxiety disorders or mood disorders or cognitive disorders in a mammal.

A further aspect of the invention provides a method of treating anxiety disorders in a mammal comprising administering to such mammal an effective amount of a compound of formula I 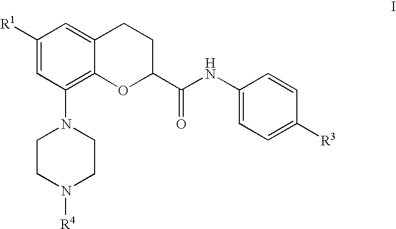
wherein:
R¹ is F or OCH₃;
R⁴is CH₃, or C₂₋₄alkyl; and
R³ is selected from morpholine or piperazine attached by nitrogen and optionally substituted on the other nitrogen (for piperazine) with —C(═O)CH₃, —CH₃, —C(═O)CH₂CH₃, —C(═O)N(CH₃)₂, —SO₂CH₂CH₃, or —SO₂CH₃,
with the proviso that when R¹ is F and R⁴ is methyl then R³ cannot be morpholine; and
with the proviso that when R¹ is F and R⁴ is methyl then R³ cannot be piperazine substituted with —CH₂OH, —CH₂CH₂OH, —SO₂CH₃, or —SO₂CH₂CH₃;
   or a pharmaceutically-acceptable salt thereof.

A further aspect of the invention provides a method of treating anxiety disorders in a mammal comprising administering to such mammal an effective amount of a compound selected from:
6-methoxy-8-(4-methylpiperazin-1-yl)-N-(4-morpholinophenyl)chroman-2-carboxamide;
(+)-6-methoxy-8-(4-methylpiperazin-1-yl)-N-(4-morpholinophenyl)chroman-2-carboxamide;
(−)-6-methoxy-8-(4-methylpiperazin-1-yl)-N-(4-morpholinophenyl)chroman-2-carboxamide;
N-(4-(4-ethanoylpiperazin-1-yl)phenyl)-6-fluoro-8-(4-methylpiperazin-1-yl)chroman-2-carboxamide;
(+)-N-(4-(4-ethanoylpiperazin-1-yl)phenyl)-6-fluoro-8-(4-methylpiperazin-1-yl)chroman-2-carboxamide;
(−)-N-(4-(4-ethanoylpiperazin-1-yl)phenyl)-6-fluoro-8-(4-methylpiperazin-1-yl)chroman-2-carboxamide;
N-(4-(4-ethanoylpiperazin-1-yl)phenyl)-6-fluoro-8-(4-methylpiperazin-1-yl)chroman-2-carboxamide;
(+)-N-(4-(4-ethanoylpiperazin-1-yl)phenyl)-6-fluoro-8-(4-methylpiperazin-1-yl)chroman-2-carboxamide;
(−)-N-(4-(4-ethanoylpiperazin-1-yl)phenyl)-6-fluoro-8-(4-methylpiperazin-1-yl)chroman-2-carboxamide;
6-fluoro-8-(4-methylpiperazin-1-yl)-N-(4-(4-propanoylpiperazin-1-yl)phenyl)chroman-2-carboxamide;
(+)-6-fluoro-8-(4-methylpiperazin-1-yl)-N-(4-(4-propanoylpiperazin-1-yl)phenyl)chroman-2-carboxamide;
(−)-6-fluoro-8-(4-methylpiperazin-1-yl)-N-(4-(4-propanoylpiperazin-1-yl)phenyl)chroman-2-carboxamide;
6-methoxy-8-(4-methylpiperazin-1-yl)-N-(4-(4-methylpiperazin-1-yl)phenyl)chroman-2-carboxamide;
(+)-6-methoxy-8-(4-methylpiperazin-1-yl)-N-(4-(4-methylpiperazin-1-yl)phenyl)chroman-2-carboxamide;
(−)-6-methoxy-8-(4-methylpiperazin-1-yl)-N-(4-(4-methylpiperazin-1-yl)phenyl)chroman-2-carboxamide;
6-fluoro-8-(4-methylpiperazin-1-yl)-N-(4-(4-methylpiperazin-1-yl)phenyl)chroman-2-carboxamide;
(+)-6-fluoro-8-(4-methylpiperazin-1-yl)-N-(4-(4-methylpiperazin-1-yl)phenyl)chroman-2-carboxamide;
(−)-6-fluoro-8-(4-methylpiperazin-1-yl)-N-(4-(4-methylpiperazin-1-yl)phenyl)chroman-2-carboxamide;
6-fluoro-8-(4-methylpiperazin-1-yl)-N-(4-(4-propanoylpiperazin-1-yl)phenyl)chroman-2-carboxamide;
(+)-6-fluoro-8-(4-methylpiperazin-1-yl)-N-(4-(4-propanoylpiperazin-1-yl)phenyl)chroman-2-carboxamide;
(−)-6-fluoro-8-(4-methylpiperazin-1-yl)-N-(4-(4-propanoylpiperazin-1-yl)phenyl)chroman-2-carboxamide;
N-(4-(4-(ethylsulfonyl)piperazin-1-yl)phenyl)-6-methoxy-8-(4-methylpiperazin-1-yl)chroman-2-carboxamide;
(+)-N-(4-(4-(ethylsulfonyl)piperazin-1-yl)phenyl)-6-methoxy-8-(4-methylpiperazin-1-yl)chroman-2-carboxamide;
(−)-N-(4-(4-(ethylsulfonyl)piperazin-1-yl)phenyl)-6-methoxy-8-(4-methylpiperazin-1-yl)chroman-2-carboxamide;
8-(4-butylpiperazin-1-yl)-6-methoxy-N-(4-morpholinophenyl)chroman-2-carboxamide;
(+)-8-(4-butylpiperazin-1-yl)-6-methoxy-N-(4-morpholinophenyl)chroman-2-carboxamide;
(−)-8-(4-butylpiperazin-1-yl)-6-methoxy-N-(4-morpholinophenyl)chroman-2-carboxamide;
6-methoxy-N-(4-morpholinophenyl)-8-(4-propylpiperazin-1-yl)chroman-2-carboxamide;
(+)-6-methoxy-N-(4-morpholinophenyl)-8-(4-propylpiperazin-1-yl)chroman-2-carboxamide;
(−)-6-methoxy-N-(4-morpholinophenyl)-8-(4-propylpiperazin-1-yl)chroman-2-carboxamide;
8-(4-isopropylpiperazin-1-yl)-6-methoxy-N-(4-morpholinophenyl)chroman-2-carboxamide;
(+)-8-(4-isopropylpiperazin-1-yl)-6-methoxy-N-(4-morpholinophenyl)chroman-2-carboxamide;
(−)-8-(4-isopropylpiperazin-1-yl)-6-methoxy-N-(4-morpholinophenyl)chroman-2-carboxamide;
8-(4-ethylpiperazin-1-yl)-6-methoxy-N-(4-morpholinophenyl)chroman-2-carboxamide;
(+)-8-(4-ethylpiperazin-1-yl)-6-methoxy-N-(4-morpholinophenyl)chroman-2-carboxamide;
(−)-8-(4-ethylpiperazin-1-yl)-6-methoxy-N-(4-morpholinophenyl)chroman-2-carboxamide;
6-methoxy-8-(4-methylpiperazin-1-yl)-N-(4-(4-propanoylpiperazin-1-yl)phenyl)chroman-2-carboxamide;
(+)-6-methoxy-8-(4-methylpiperazin-1-yl)-N-(4-(4-propanoylpiperazin-1-yl)phenyl)chroman-2-carboxamide;
(−)-6-methoxy-8-(4-methylpiperazin-1-yl)-N-(4-(4-propanoylpiperazin-1-yl)phenyl)chroman-2-carboxamide;
4-(4-(6-methoxy-8-(4-methylpiperazin-1-yl)chroman-2-carboxamido)phenyl)-N,N-dimethylpiperazine-1-carboxamide;
(+)-4-(4-(6-methoxy-8-(4-methylpiperazin-1-yl)chroman-2-carboxamido)phenyl)-N,N-dimethylpiperazine-1-carboxamide;
(−)-4-(4-(6-methoxy-8-(4-methylpiperazin-1-yl)chroman-2-carboxamido)phenyl)-N,N-dimethylpiperazine-1-carboxamide;
4-(4-(6-methoxy-8-(4-methylpiperazin-1-yl)chroman-2-carboxamido)phenyl)-N,N-dimethylpiperazine-1-carboxamide;
(+)-4-(4-(6-methoxy-8-(4-methylpiperazin-1-yl)chroman-2-carboxamido)phenyl)-N,N-dimethylpiperazine-1-carboxamide;
(−)-4-(4-(6-methoxy-8-(4-methylpiperazin-1-yl)chroman-2-carboxamido)phenyl)-N,N-dimethylpiperazine-1-carboxamide;
4-(4-(6-fluoro-8-(4-methylpiperazin-1-yl)chroman-2-carboxamido)phenyl)-N,N-dimethylpiperazine-1-carboxamide;
(+)-4-(4-(6-fluoro-8-(4-methylpiperazin-1-yl)chroman-2-carboxamido)phenyl)-N,N-dimethylpiperazine-1-carboxamide;
(−)-4-(4-(6-fluoro-8-(4-methylpiperazin-1-yl)chroman-2-carboxamido)phenyl)-N,N-dimethylpiperazine-1-carboxamide;
6-methoxy-8-(4-methylpiperazin-1-yl)-N-(4-(4-(methylsulfonyl)piperazin-1-yl)phenyl)chroman-2-carboxamide;
(+)-6-methoxy-8-(4-methylpiperazin-1-yl)-N-(4-(4-(methylsulfonyl)piperazin-1-yl)phenyl)chroman-2-carboxamide;
(−)-6-methoxy-8-(4-methylpiperazin-1-yl)-N-(4-(4-(methylsulfonyl)piperazin-1-yl)phenyl)chroman-2-carboxamide;
N-(4-(4-ethanoylpiperazin-1-yl)phenyl)-6-methoxy-8-(4-methylpiperazin-1-yl)chroman-2-carboxamide;
(+)-N-(4-(4-ethanoylpiperazin-1-yl)phenyl)-6-methoxy-8-(4-methylpiperazin-1-yl)chroman-2-carboxamide;
(−)-N-(4-(4-ethanoylpiperazin-1-yl)phenyl)-6-methoxy-8-(4-methylpiperazin-1-yl)chroman-2-carboxamide;
6-methoxy-8-(4-methylpiperazin-1-yl)-N-(4-(4-propanoylpiperazin-1-yl)phenyl)chroman-2-carboxamide;
(+) 6-methoxy-8-(4-methylpiperazin-1-yl)-N-(4-(4-propanoylpiperazin-1-yl)phenyl)chroman-2-carboxamide;
(−)-6-methoxy-8-(4-methylpiperazin-1-yl)-N-(4-(4-propanoylpiperazin-1-yl)phenyl)chroman-2-carboxamide;
N-(4-(4-ethanoylpiperazin-1-yl)phenyl)-6-methoxy-8-(4-methylpiperazin-1-yl)chroman-2-carboxamide;
(+)-N-(4-(4-ethanoylpiperazin-1-yl)phenyl)-6-methoxy-8-(4-methylpiperazin-1-yl)chroman-2-carboxamide; and
(−)-N-(4-(4-ethanoylpiperazin-1-yl)phenyl)-6-methoxy-8-(4-methylpiperazin-1-yl)chroman-2-carboxamide;
   or a pharmaceutically-acceptable salt thereof.

A further aspect of the invention provides a method of treating anxiety disorders in a mammal comprising administering to such mammal an effective amount of a compound selected from:
(+)-6-methoxy-8-(4-methylpiperazin-1-yl)-N-(4-morpholinophenyl)chroman-2-carboxamide;
(+)-N-(4-(4-ethanoylpiperazin-1-yl)phenyl)-6-fluoro-8-(4-methylpiperazin-1-yl)chroman-2-carboxamide;
(+)-N-(4-(4-ethanoylpiperazin-1-yl)phenyl)-6-fluoro-8-(4-methylpiperazin-1-yl)chroman-2-carboxamide;
(+)-6-fluoro-8-(4-methylpiperazin-1-yl)-N-(4-(4-propanoylpiperazin-1-yl)phenyl)chroman-2-carboxamide;
(+)-6-methoxy-8-(4-methylpiperazin-1-yl)-N-(4-(4-methylpiperazin-1-yl)phenyl)chroman-2-carboxamide;
(+)-6-fluoro-8-(4-methylpiperazin-1-yl)-N-(4-(4-methylpiperazin-1-yl)phenyl)chroman-2-carboxamide;
(+)-6-fluoro-8-(4-methylpiperazin-1-yl)-N-(4-(4-propanoylpiperazin-1-yl)phenyl)chroman-2-carboxamide;
)-N-(4-(4-(ethylsulfonyl)piperazin-1-yl)phenyl)-6-methoxy-8-(4-methylpiperazin-1-yl)chroman-2-carboxamide
(+)-8-(4-butylpiperazin-1-yl)-6-methoxy-N-(4-morpholinophenyl)chroman-2-carboxamide;
(+)-6-methoxy-N-(4-morpholinophenyl)-8-(4-propylpiperazin-1-yl)chroman-2-carboxamide;
(+)-8-(4-isopropylpiperazin-1-yl)-6-methoxy-N-(4-morpholinophenyl)chroman-2-carboxamide;
(+)-8-(4-ethylpiperazin-1-yl)-6-methoxy-N-(4-morpholinophenyl)chroman-2-carboxamide;
(+)-6-methoxy-8-(4-methylpiperazin-1-yl)-N-(4-(4-propanoylpiperazin-1-yl)phenyl)chroman-2-carboxamide;
(+)-4-(4-(6-methoxy-8-(4-methylpiperazin-1-yl)chroman-2-carboxamido)phenyl)-N,N-dimethylpiperazine-1-carboxamide;
(+)-4-(4-(6-methoxy-8-(4-methylpiperazin-1-yl)chroman-2-carboxamido)phenyl)-N,N-dimethylpiperazine-1-carboxamide;
(+)-4-(4-(6-fluoro-8-(4-methylpiperazin-1-yl)chroman-2-carboxamido)phenyl)-N,N-dimethylpiperazine-1-carboxamide;
(+)-6-methoxy-8-(4-methylpiperazin-1-yl)-N-(4-(4-(methylsulfonyl)piperazin-1-yl)phenyl)chroman-2-carboxamide;
(+)-N-(4-(4-ethanoylpiperazin-1-yl)phenyl)-6-methoxy-8-(4-methylpiperazin-1-yl)chroman-2-carboxamide;
(+) 6-methoxy-8-(4-methylpiperazin-1-yl)-N-(4-(4-propanoylpiperazin-1-yl)phenyl)chroman-2-carboxamide; and
(+)-N-(4-(4-ethanoylpiperazin-1-yl)phenyl)-6-methoxy-8-(4-methylpiperazin-1-yl)chroman-2-carboxamide;
   or a pharmaceutically-acceptable salt thereof.

A further aspect of the invention provides a method of treating mood disorders in a mammal comprising administering to such mammal an effective amount of a compound of formula I 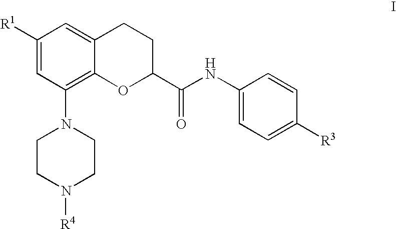
wherein:
R¹ is F or OCH₃;
R⁴ is CH₃, or C₂₋₄alkyl; and
R³ is selected from morpholine or piperazine attached by nitrogen and optionally substituted on the other nitrogen (for piperazine) with —C(═O)CH₃, —CH₃, —C(═O)CH₂CH₃, —C(═O)N(CH₃)₂, —SO₂CH₂CH₃, or —SO₂CH₃,
with the proviso that when R¹ is F and R⁴ is methyl then R³ cannot be morpholine; and
with the proviso that when R¹ is F and R⁴ is methyl then R³ cannot be piperazine substituted with —CH₂OH, —CH₂CH₂OH, —SO₂CH₃, or —SO₂CH₂CH₃;
   or a pharmaceutically-acceptable salt thereof.

A further aspect of the invention provides a method of treating mood disorders in a mammal comprising administering to such mammal an effective amount of a compound selected from:
6-methoxy-8-(4-methylpiperazin-1-yl)-N-(4-morpholinophenyl)chroman-2-carboxamide;
(+)-6-methoxy-8-(4-methylpiperazin-1-yl)-N-(4-morpholinophenyl)chroman-2-carboxamide;
(−)-6-methoxy-8-(4-methylpiperazin-1-yl)-N-(4-morpholinophenyl)chroman-2-carboxamide;
N-(4-(4-ethanoylpiperazin-1-yl)phenyl)-6-fluoro-8-(4-methylpiperazin-1-yl)chroman-2-carboxamide;
(+)-N-(4-(4-ethanoylpiperazin-1-yl)phenyl)-6-fluoro-8-(4-methylpiperazin-1-yl)chroman-2-carboxamide;
(−)-N-(4-(4-ethanoylpiperazin-1-yl)phenyl)-6-fluoro-8-(4-methylpiperazin-1-yl)chroman-2-carboxamide;
N-(4-(4-ethanoylpiperazin-1-yl)phenyl)-6-fluoro-8-(4-methylpiperazin-1-yl)chroman-2-carboxamide;
(+)-N-(4-(4-ethanoylpiperazin-1-yl)phenyl)-6-fluoro-8-(4-methylpiperazin-1-yl)chroman-2-carboxamide;
(−)-N-(4-(4-ethanoylpiperazin-1-yl)phenyl)-6-fluoro-8-(4-methylpiperazin-1-yl)chroman-2-carboxamide;
6-fluoro-8-(4-methylpiperazin-1-yl)-N-(4-(4-propanoylpiperazin-1-yl)phenyl)chroman-2-carboxamide;
(+)-6-fluoro-8-(4-methylpiperazin-1-yl)-N-(4-(4-propanoylpiperazin-1-yl)phenyl)chroman-2-carboxamide;
(−)-6-fluoro-8-(4-methylpiperazin-1-yl)-N-(4-(4-propanoylpiperazin-1-yl)phenyl)chroman-2-carboxamide;
6-methoxy-8-(4-methylpiperazin-1-yl)-N-(4-(4-methylpiperazin-1-yl)phenyl)chroman-2-carboxamide;
(+)-6-methoxy-8-(4-methylpiperazin-1-yl)-N-(4-(4-methylpiperazin-1-yl)phenyl)chroman-2-carboxamide;
(−)-6-methoxy-8-(4-methylpiperazin-1-yl)-N-(4-(4-methylpiperazin-1-yl)phenyl)chroman-2-carboxamide;
6-fluoro-8-(4-methylpiperazin-1-yl)-N-(4-(4-methylpiperazin-1-yl)phenyl)chroman-2-carboxamide;
(+)-6-fluoro-8-(4-methylpiperazin-1-yl)-N-(4-(4-methylpiperazin-1-yl)phenyl)chroman-2-carboxamide;
(−)-6-fluoro-8-(4-methylpiperazin-1-yl)-N-(4-(4-methylpiperazin-1-yl)phenyl)chroman-2-carboxamide;
6-fluoro-8-(4-methylpiperazin-1-yl)-N-(4-(4-propanoylpiperazin-1-yl)phenyl)chroman-2-carboxamide;
(+)-6-fluoro-8-(4-methylpiperazin-1-yl)-N-(4-(4-propanoylpiperazin-1-yl)phenyl)chroman-2-carboxamide;
(−)-6-fluoro-8-(4-methylpiperazin-1-yl)-N-(4-(4-propanoylpiperazin-1-yl)phenyl)chroman-2-carboxamide;
N-(4-(4-(ethylsulfonyl)piperazin-1-yl)phenyl)-6-methoxy-8-(4-methylpiperazin-1-yl)chroman-2-carboxamide;
(+)-N-(4-(4-(ethylsulfonyl)piperazin-1-yl)phenyl)-6-methoxy-8-(4-methylpiperazin-1-yl)chroman-2-carboxamide;
(−)-N-(4-(4-(ethylsulfonyl)piperazin-1-yl)phenyl)-6-methoxy-8-(4-methylpiperazin-1-yl)chroman-2-carboxamide;
8-(4-butylpiperazin-1-yl)-6-methoxy-N-(4-morpholinophenyl)chroman-2-carboxamide;
(+)-8-(4-butylpiperazin-1-yl)-6-methoxy-N-(4-morpholinophenyl)chroman-2-carboxamide;
(−)-8-(4-butylpiperazin-1-yl)-6-methoxy-N-(4-morpholinophenyl)chroman-2-carboxamide;
6-methoxy-N-(4-morpholinophenyl)-8-(4-propylpiperazin-1-yl)chroman-2-carboxamide;
(+)-6-methoxy-N-(4-morpholinophenyl)-8-(4-propylpiperazin-1-yl)chroman-2-carboxamide;
(−)-6-methoxy-N-(4-morpholinophenyl)-8-(4-propylpiperazin-1-yl)chroman-2-carboxamide;
8-(4-isopropylpiperazin-1-yl)-6-methoxy-N-(4-morpholinophenyl)chroman-2-carboxamide;
(+)-8-(4-isopropylpiperazin-1-yl)-6-methoxy-N-(4-morpholinophenyl)chroman-2-carboxamide;
(−)-8-(4-isopropylpiperazin-1-yl)-6-methoxy-N-(4-morpholinophenyl)chroman-2-carboxamide;
8-(4-ethylpiperazin-1-yl)-6-methoxy-N-(4-morpholinophenyl)chroman-2-carboxamide;
(+)-8-(4-ethylpiperazin-1-yl)-6-methoxy-N-(4-morpholinophenyl)chroman-2-carboxamide;
(−)-8-(4-ethylpiperazin-1-yl)-6-methoxy-N-(4-morpholinophenyl)chroman-2-carboxamide;
6-methoxy-8-(4-methylpiperazin-1-yl)-N-(4-(4-propanoylpiperazin-1-yl)phenyl)chroman-2-carboxamide;
(+)-6-methoxy-8-(4-methylpiperazin-1-yl)-N-(4-(4-propanoylpiperazin-1-yl)phenyl)chroman-2-carboxamide;
(−)-6-methoxy-8-(4-methylpiperazin-1-yl)-N-(4-(4-propanoylpiperazin-1-yl)phenyl)chroman-2-carboxamide;
4-(4-(6-methoxy-8-(4-methylpiperazin-1-yl)chroman-2-carboxamido)phenyl)-N,N-dimethylpiperazine-1-carboxamide;
(+)-4-(4-(6-methoxy-8-(4-methylpiperazin-1-yl)chroman-2-carboxamido)phenyl)-N,N-dimethylpiperazine-1-carboxamide;
(−)-4-(4-(6-methoxy-8-(4-methylpiperazin-1-yl)chroman-2-carboxamido)phenyl)-N,N-dimethylpiperazine-1-carboxamide;
4-(4-(6-methoxy-8-(4-methylpiperazin-1-yl)chroman-2-carboxamido)phenyl)-N,N-dimethylpiperazine-1-carboxamide;
(+)-4-(4-(6-methoxy-8-(4-methylpiperazin-1-yl)chroman-2-carboxamido)phenyl)-N,N-dimethylpiperazine-1-carboxamide;
(−)-4-(4-(6-methoxy-8-(4-methylpiperazin-1-yl)chroman-2-carboxamido)phenyl)-N,N-dimethylpiperazine-1-carboxamide;
4-(4-(6-fluoro-8-(4-methylpiperazin-1-yl)chroman-2-carboxamido)phenyl)-N,N-dimethylpiperazine-1-carboxamide;
(+)-4-(4-(6-fluoro-8-(4-methylpiperazin-1-yl)chroman-2-carboxamido)phenyl)-N,N-dimethylpiperazine-1-carboxamide;
(−)-4-(4-(6-fluoro-8-(4-methylpiperazin-1-yl)chroman-2-carboxamido)phenyl)-N,N-dimethylpiperazine-1-carboxamide;
6-methoxy-8-(4-methylpiperazin-1-yl)-N-(4-(4-(methylsulfonyl)piperazin-1-yl)phenyl)chroman-2-carboxamide;
(+)-6-methoxy-8-(4-methylpiperazin-1-yl)-N-(4-(4-(methylsulfonyl)piperazin-1-yl)phenyl)chroman-2-carboxamide;
(−)-6-methoxy-8-(4-methylpiperazin-1-yl)-N-(4-(4-(methylsulfonyl)piperazin-1-yl)phenyl)chroman-2-carboxamide;
N-(4-(4-ethanoylpiperazin-1-yl)phenyl)-6-methoxy-8-(4-methylpiperazin-1-yl)chroman-2-carboxamide;
(+)-N-(4-(4-ethanoylpiperazin-1-yl)phenyl)-6-methoxy-8-(4-methylpiperazin-1-yl)chroman-2-carboxamide;
(−)-N-(4-(4-ethanoylpiperazin-1-yl)phenyl)-6-methoxy-8-(4-methylpiperazin-1-yl)chroman-2-carboxamide;
6-methoxy-8-(4-methylpiperazin-1-yl)-N-(4-(4-propanoylpiperazin-1-yl)phenyl)chroman-2-carboxamide;
(+) 6-methoxy-8-(4-methylpiperazin-1-yl)-N-(4-(4-propanoylpiperazin-1-yl)phenyl)chroman-2-carboxamide;
(−)-6-methoxy-8-(4-methylpiperazin-1-yl)-N-(4-(4-propanoylpiperazin-1-yl)phenyl)chroman-2-carboxamide;
N-(4-(4-ethanoylpiperazin-1-yl)phenyl)-6-methoxy-8-(4-methylpiperazin-1-yl)chroman-2-carboxamide;
(+)-N-(4-(4-ethanoylpiperazin-1-yl)phenyl)-6-methoxy-8-(4-methylpiperazin-1-yl)chroman-2-carboxamide; and
(−)-N-(4-(4-ethanoylpiperazin-1-yl)phenyl)-6-methoxy-8-(4-methylpiperazin-1-yl)chroman-2-carboxamide;
   or a pharmaceutically-acceptable salt thereof.

A further aspect of the invention provides a method of treating mood disorders in a mammal comprising administering to such mammal an effective amount of a compound selected from:
(+)-6-methoxy-8-(4-methylpiperazin-1-yl)-N-(4-morpholinophenyl)chroman-2-carboxamide;
(+)-N-(4-(4-ethanoylpiperazin-1-yl)phenyl)-6-fluoro-8-(4-methylpiperazin-1-yl)chroman-2-carboxamide;
(+)-N-(4-(4-ethanoylpiperazin-1-yl)phenyl)-6-fluoro-8-(4-methylpiperazin-1-yl)chroman-2-carboxamide;
(+)-6-fluoro-8-(4-methylpiperazin-1-yl)-N-(4-(4-propanoylpiperazin-1-yl)phenyl)chroman-2-carboxamide;
(+)-6-methoxy-8-(4-methylpiperazin-1-yl)-N-(4-(4-methylpiperazin-1-yl)phenyl)chroman-2-carboxamide;
(+)-6-fluoro-8-(4-methylpiperazin-1-yl)-N-(4-(4-methylpiperazin-1-yl)phenyl)chroman-2-carboxamide;
(+)-6-fluoro-8-(4-methylpiperazin-1-yl)-N-(4-(4-propanoylpiperazin-1-yl)phenyl)chroman-2-carboxamide;
(+)-N-(4-(4-(ethylsulfonyl)piperazin-1-yl)phenyl)-6-methoxy-8-(4-methylpiperazin-1-yl)chroman-2-carboxamide
(+)-8-(4-butylpiperazin-1-yl)-6-methoxy-N-(4-morpholinophenyl)chroman-2-carboxamide;
(+)-6-methoxy-N-(4-morpholinophenyl)-8-(4-propylpiperazin-1-yl)chroman-2-carboxamide;
(+)-8-(4-isopropylpiperazin-1-yl)-6-methoxy-N-(4-morpholinophenyl)chroman-2-carboxamide;
(+)-8-(4-ethylpiperazin-1-yl)-6-methoxy-N-(4-morpholinophenyl)chroman-2-carboxamide;
(+)-6-methoxy-8-(4-methylpiperazin-1-yl)-N-(4-(4-propanoylpiperazin-1-yl)phenyl)chroman-2-carboxamide;
(+)-4-(4-(6-methoxy-8-(4-methylpiperazin-1-yl)chroman-2-carboxamido)phenyl)-N,N-dimethylpiperazine-1-carboxamide;
(+)-4-(4-(6-methoxy-8-(4-methylpiperazin-1-yl)chroman-2-carboxamido)phenyl)-N,N-dimethylpiperazine-1-carboxamide;
(+)-4-(4-(6-fluoro-8-(4-methylpiperazin-1-yl)chroman-2-carboxamido)phenyl)-N,N-dimethylpiperazine-1-carboxamide;
(+)-6-methoxy-8-(4-methylpiperazin-1-yl)-N-(4-(4-(methylsulfonyl)piperazin-1-yl)phenyl)chroman-2-carboxamide;
(+)-N-(4-(4-ethanoylpiperazin-1-yl)phenyl)-6-methoxy-8-(4-methylpiperazin-1-yl)chroman-2-carboxamide;
(+) 6-methoxy-8-(4-methylpiperazin-1-yl)-N-(4-(4-propanoylpiperazin-1-yl)phenyl)chroman-2-carboxamide; and
(+)-N-(4-(4-ethanoylpiperazin-1-yl)phenyl)-6-methoxy-8-(4-methylpiperazin-1-yl)chroman-2-carboxamide;
   or a pharmaceutically-acceptable salt thereof.

A further aspect of the invention provides a method of treating cognitive disorders in a mammal comprising administering to such mammal an effective amount of a compound of formula I 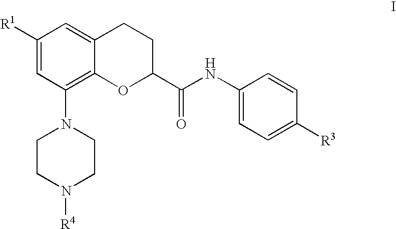
wherein:
R¹ is F or OCH₃;
R⁴ is CH₃, or C₂₋₄alkyl; and
R³ is selected from morpholine or piperazine attached by nitrogen and optionally substituted on the other nitrogen (for piperazine) with —C(═O)CH₃, —CH₃, —C(═O)CH₂CH₃, —C(═O)N(CH₃)₂, —SO₂CH₂CH₃, or —SO₂CH₃,
with the proviso that when R¹ is F and R⁴ is methyl then R³ cannot be morpholine; and
with the proviso that when R¹ is F and R⁴ is methyl then R³ cannot be piperazine substituted with —CH₂OH, —CH₂CH₂OH, —SO₂CH₃, or —SO₂CH₂CH₃;
   or a pharmaceutically-acceptable salt thereof.

A further aspect of the invention provides a method of treating cognitive disorders in a mammal comprising administering to such mammal an effective amount of a compound selected from:
6-methoxy-8-(4-methylpiperazin-1-yl)-N-(4-morpholinophenyl)chroman-2-carboxamide;
(+)-6-methoxy-8-(4-methylpiperazin-1-yl)-N-(4-morpholinophenyl)chroman-2-carboxamide;
(−)-6-methoxy-8-(4-methylpiperazin-1-yl)-N-(4-morpholinophenyl)chroman-2-carboxamide;
N-(4-(4-ethanoylpiperazin-1-yl)phenyl)-6-fluoro-8-(4-methylpiperazin-1-yl)chroman-2-carboxamide;
(+)-N-(4-(4-ethanoylpiperazin-1-yl)phenyl)-6-fluoro-8-(4-methylpiperazin-1-yl)chroman-2-carboxamide;
(−)-N-(4-(4-ethanoylpiperazin-1-yl)phenyl)-6-fluoro-8-(4-methylpiperazin-1-yl)chroman-2-carboxamide;
N-(4-(4-ethanoylpiperazin-1-yl)phenyl)-6-fluoro-8-(4-methylpiperazin-1-yl)chroman-2-carboxamide;
(+)-N-(4-(4-ethanoylpiperazin-1-yl)phenyl)-6-fluoro-8-(4-methylpiperazin-1-yl)chroman-2-carboxamide;
(−)-N-(4-(4-ethanoylpiperazin-1-yl)phenyl)-6-fluoro-8-(4-methylpiperazin-1-yl)chroman-2-carboxamide;
6-fluoro-8-(4-methylpiperazin-1-yl)-N-(4-(4-propanoylpiperazin-1-yl)phenyl)chroman-2-carboxamide;
(+)-6-fluoro-8-(4-methylpiperazin-1-yl)-N-(4-(4-propanoylpiperazin-1-yl)phenyl)chroman-2-carboxamide;
(−)-6-fluoro-8-(4-methylpiperazin-1-yl)-N-(4-(4-propanoylpiperazin-1-yl)phenyl)chroman-2-carboxamide;
6-methoxy-8-(4-methylpiperazin-1-yl)-N-(4-(4-methylpiperazin-1-yl)phenyl)chroman-2-carboxamide;
(+)-6-methoxy-8-(4-methylpiperazin-1-yl)-N-(4-(4-methylpiperazin-1-yl)phenyl)chroman-2-carboxamide;
(−)-6-methoxy-8-(4-methylpiperazin-1-yl)-N-(4-(4-methylpiperazin-1-yl)phenyl)chroman-2-carboxamide;
6-fluoro-8-(4-methylpiperazin-1-yl)-N-(4-(4-methylpiperazin-1-yl)phenyl)chroman-2-carboxamide;
(+)-6-fluoro-8-(4-methylpiperazin-1-yl)-N-(4-(4-methylpiperazin-1-yl)phenyl)chroman-2-carboxamide;
(−)-6-fluoro-8-(4-methylpiperazin-1-yl)-N-(4-(4-methylpiperazin-1-yl)phenyl)chroman-2-carboxamide;
6-fluoro-8-(4-methylpiperazin-1-yl)-N-(4-(4-propanoylpiperazin-1-yl)phenyl)chroman-2-carboxamide;
(+)-6-fluoro-8-(4-methylpiperazin-1-yl)-N-(4-(4-propanoylpiperazin-1-yl)phenyl)chroman-2-carboxamide;
(−)-6-fluoro-8-(4-methylpiperazin-1-yl)-N-(4-(4-propanoylpiperazin-1-yl)phenyl)chroman-2-carboxamide;
N-(4-(4-(ethylsulfonyl)piperazin-1-yl)phenyl)-6-methoxy-8-(4-methylpiperazin-1-yl)chroman-2-carboxamide;
(+)-N-(4-(4-(ethylsulfonyl)piperazin-1-yl)phenyl)-6-methoxy-8-(4-methylpiperazin-1-yl)chroman-2-carboxamide;
(−)-N-(4-(4-(ethylsulfonyl)piperazin-1-yl)phenyl)-6-methoxy-8-(4-methylpiperazin-1-yl)chroman-2-carboxamide;
8-(4-butylpiperazin-1-yl)-6-methoxy-N-(4-morpholinophenyl)chroman-2-carboxamide;
(+)-8-(4-butylpiperazin-1-yl)-6-methoxy-N-(4-morpholinophenyl)chroman-2-carboxamide;
(−)-8-(4-butylpiperazin-1-yl)-6-methoxy-N-(4-morpholinophenyl)chroman-2-carboxamide;
6-methoxy-N-(4-morpholinophenyl)-8-(4-propylpiperazin-1-yl)chroman-2-carboxamide;
(+)-6-methoxy-N-(4-morpholinophenyl)-8-(4-propylpiperazin-1-yl)chroman-2-carboxamide;
(−)-6-methoxy-N-(4-morpholinophenyl)-8-(4-propylpiperazin-1-yl)chroman-2-carboxamide;
8-(4-isopropylpiperazin-1-yl)-6-methoxy-N-(4-morpholinophenyl)chroman-2-carboxamide;
(+)-8-(4-isopropylpiperazin-1-yl)-6-methoxy-N-(4-morpholinophenyl)chroman-2-carboxamide;
(−)-8-(4-isopropylpiperazin-1-yl)-6-methoxy-N-(4-morpholinophenyl)chroman-2-carboxamide;
8-(4-ethylpiperazin-1-yl)-6-methoxy-N-(4-morpholinophenyl)chroman-2-carboxamide;
(+)-8-(4-ethylpiperazin-1-yl)-6-methoxy-N-(4-morpholinophenyl)chroman-2-carboxamide;
(−)-8-(4-ethylpiperazin-1-yl)-6-methoxy-N-(4-morpholinophenyl)chroman-2-carboxamide;
6-methoxy-8-(4-methylpiperazin-1-yl)-N-(4-(4-propanoylpiperazin-1-yl)phenyl)chroman-2-carboxamide;
(+)-6-methoxy-8-(4-methylpiperazin-1-yl)-N-(4-(4-propanoylpiperazin-1-yl)phenyl)chroman-2-carboxamide;
(−)-6-methoxy-8-(4-methylpiperazin-1-yl)-N-(4-(4-propanoylpiperazin-1-yl)phenyl)chroman-2-carboxamide;
4-(4-(6-methoxy-8-(4-methylpiperazin-1-yl)chroman-2-carboxamido)phenyl)-N,N-dimethylpiperazine-1-carboxamide;
(+)-4-(4-(6-methoxy-8-(4-methylpiperazin-1-yl)chroman-2-carboxamido)phenyl)-N,N-dimethylpiperazine-1-carboxamide;
(−)-4-(4-(6-methoxy-8-(4-methylpiperazin-1-yl)chroman-2-carboxamido)phenyl)-N,N-dimethylpiperazine-1-carboxamide;
4-(4-(6-methoxy-8-(4-methylpiperazin-1-yl)chroman-2-carboxamido)phenyl)-N,N-dimethylpiperazine-1-carboxamide;
(+)-4-(4-(6-methoxy-8-(4-methylpiperazin-1-yl)chroman-2-carboxamido)phenyl)-N,N-dimethylpiperazine-1-carboxamide;
(−)-4-(4-(6-methoxy-8-(4-methylpiperazin-1-yl)chroman-2-carboxamido)phenyl)-N,N-dimethylpiperazine-1-carboxamide;
4-(4-(6-fluoro-8-(4-methylpiperazin-1-yl)chroman-2-carboxamido)phenyl)-N,N-dimethylpiperazine-1-carboxamide;
(+)-4-(4-(6-fluoro-8-(4-methylpiperazin-1-yl)chroman-2-carboxamido)phenyl)-N,N-dimethylpiperazine-1-carboxamide;
(−)-4-(4-(6-fluoro-8-(4-methylpiperazin-1-yl)chroman-2-carboxamido)phenyl)-N,N-dimethylpiperazine-1-carboxamide;
6-methoxy-8-(4-methylpiperazin-1-yl)-N-(4-(4-(methylsulfonyl)piperazin-1-yl)phenyl)chroman-2-carboxamide;
(+)-6-methoxy-8-(4-methylpiperazin-1-yl)-N-(4-(4-(methylsulfonyl)piperazin-1-yl)phenyl)chroman-2-carboxamide;
(−)-6-methoxy-8-(4-methylpiperazin-1-yl)-N-(4-(4-(methylsulfonyl)piperazin-1-yl)phenyl)chroman-2-carboxamide;
N-(4-(4-ethanoylpiperazin-1-yl)phenyl)-6-methoxy-8-(4-methylpiperazin-1-yl)chroman-2-carboxamide;
(+)-N-(4-(4-ethanoylpiperazin-1-yl)phenyl)-6-methoxy-8-(4-methylpiperazin-1-yl)chroman-2-carboxamide;
(−)-N-(4-(4-ethanoylpiperazin-1-yl)phenyl)-6-methoxy-8-(4-methylpiperazin-1-yl)chroman-2-carboxamide;
6-methoxy-8-(4-methylpiperazin-1-yl)-N-(4-(4-propanoylpiperazin-1-yl)phenyl)chroman-2-carboxamide;
(+) 6-methoxy-8-(4-methylpiperazin-1-yl)-N-(4-(4-propanoylpiperazin-1-yl)phenyl)chroman-2-carboxamide;
(−)-6-methoxy-8-(4-methylpiperazin-1-yl)-N-(4-(4-propanoylpiperazin-1-yl)phenyl)chroman-2-carboxamide;
N-(4-(4-ethanoylpiperazin-1-yl)phenyl)-6-methoxy-8-(4-methylpiperazin-1-yl)chroman-2-carboxamide;
(+)-N-(4-(4-ethanoylpiperazin-1-yl)phenyl)-6-methoxy-8-(4-methylpiperazin-1-yl)chroman-2-carboxamide; and
(−)-N-(4-(4-ethanoylpiperazin-1-yl)phenyl)-6-methoxy-8-(4-methylpiperazin-1-yl)chroman-2-carboxamide;
   or a pharmaceutically-acceptable salt thereof.

A further aspect of the invention provides a method of treating cognitive disorders in a mammal comprising administering to such mammal an effective amount of a compound selected from:
(+)-6-methoxy-8-(4-methylpiperazin-1-yl)-N-(4-morpholinophenyl)chroman-2-carboxamide;
(+)-N-(4-(4-ethanoylpiperazin-1-yl)phenyl)-6-fluoro-8-(4-methylpiperazin-1-yl)chroman-2-carboxamide;
(+)-N-(4-(4-ethanoylpiperazin-1-yl)phenyl)-6-fluoro-8-(4-methylpiperazin-1-yl)chroman-2-carboxamide;
(+)-6-fluoro-8-(4-methylpiperazin-1-yl)-N-(4-(4-propanoylpiperazin-1-yl)phenyl)chroman-2-carboxamide;
(+)-6-methoxy-8-(4-methylpiperazin-1-yl)-N-(4-(4-methylpiperazin-1-yl)phenyl)chroman-2-carboxamide;
(+)-6-fluoro-8-(4-methylpiperazin-1-yl)-N-(4-(4-methylpiperazin-1-yl)phenyl)chroman-2-carboxamide;
(+)-6-fluoro-8-(4-methylpiperazin-1-yl)-N-(4-(4-propanoylpiperazin-1-yl)phenyl)chroman-2-carboxamide;
(+)-N-(4-(4-(ethylsulfonyl)piperazin-1-yl)phenyl)-6-methoxy-8-(4-methylpiperazin-1-yl)chroman-2-carboxamide
(+)-8-(4-butylpiperazin-1-yl)-6-methoxy-N-(4-morpholinophenyl)chroman-2-carboxamide;
(+)-6-methoxy-N-(4-morpholinophenyl)-8-(4-propylpiperazin-1-yl)chroman-2-carboxamide;
(+)-8-(4-isopropylpiperazin-1-yl)-6-methoxy-N-(4-morpholinophenyl)chroman-2-carboxamide;
(+)-8-(4-ethylpiperazin-1-yl)-6-methoxy-N-(4-morpholinophenyl)chroman-2-carboxamide;
(+)-6-methoxy-8-(4-methylpiperazin-1-yl)-N-(4-(4-propanoylpiperazin-1-yl)phenyl)chroman-2-carboxamide;
(+)-4-(4-(6-methoxy-8-(4-methylpiperazin-1-yl)chroman-2-carboxamido)phenyl)-N,N-dimethylpiperazine-1-carboxamide;
(+)-4-(4-(6-methoxy-8-(4-methylpiperazin-1-yl)chroman-2-carboxamido)phenyl)-N,N-dimethylpiperazine-1-carboxamide;
(+)-4-(4-(6-fluoro-8-(4-methylpiperazin-1-yl)chroman-2-carboxamido)phenyl)-N,N-dimethylpiperazine-1-carboxamide;
(+)-6-methoxy-8-(4-methylpiperazin-1-yl)-N-(4-(4-(methylsulfonyl)piperazin-1-yl)phenyl)chroman-2-carboxamide;
(+)-N-(4-(4-ethanoylpiperazin-1-yl)phenyl)-6-methoxy-8-(4-methylpiperazin-1-yl)chroman-2-carboxamide;
(+) 6-methoxy-8-(4-methylpiperazin-1-yl)-N-(4-(4-propanoylpiperazin-1-yl)phenyl)chroman-2-carboxamide; and
(+)-N-(4-(4-ethanoylpiperazin-1-yl)phenyl)-6-methoxy-8-(4-methylpiperazin-1-yl)chroman-2-carboxamide;
   or a pharmaceutically-acceptable salt thereof.

A further aspect of the invention provides the use of a compound according to Formula I in the preparation of a medicament for the treatment of anxiety disorders or mood disorders or cognitive disorders.

A further aspect of the invention provides a pharmaceutical composition comprising a compound according to Formula I or a pharmaceutically acceptable salt thereof and a pharmaceutically acceptable carrier.

A further aspect of the invention provides a process for the preparation of a compound according to Formula I or a pharmaceutically acceptable salt thereof, which process comprises reacting a compound of formula (II): 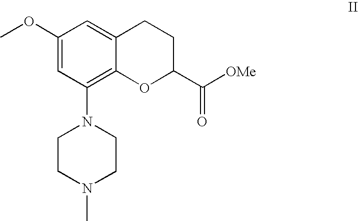
with lithium hydroxide to yield a compound of formula (III): 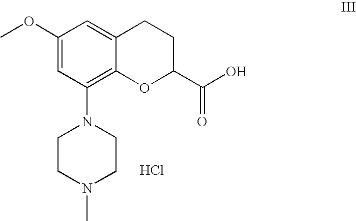
to which can be added an appropriate aniline to achieve the desired compound as shown in formula IV.

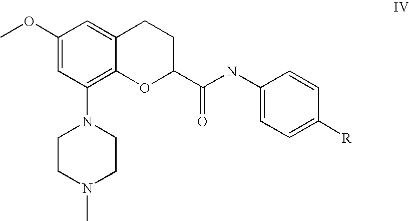

Compounds of the invention have a chiral center at the 2-position of the chroman structure. Such compounds exist in S-and R-forms illustrated below:

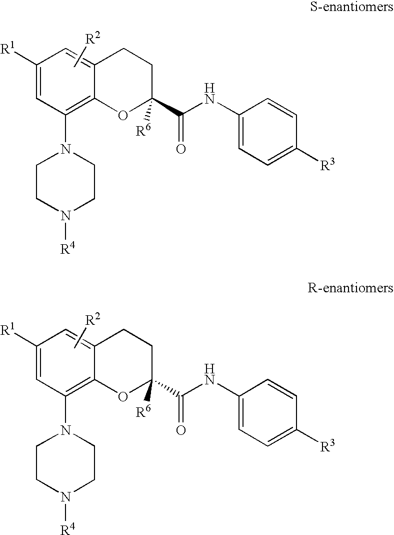

Such forms may be fractionated by chiral chromatography and it is believed that following fractionation that a dextrorotatory compound has greater antagonist activity than a laevorotatory compound. While not wishing to be bound by any theory it is currently believed that the (+) isomers are the (R) entantiomers and the (−) isomers are the (S) entantiomers. Thus, while dextrorotatory, (D) or (+) or (R), and laevorotatory, (L) or (−) compounds, are compounds of the invention, particular compounds of the invention are dextrorotatory, (D) or (+), compounds.

Particular compounds described herein illustrate, but do not limit the invention, other compounds within the scope of the invention will be apparent to those of skill in the art upon contemplation of the processes, methods and compounds described herein.

The compounds provided herein are useful in the form as a free base, but may also be provided in the form of a pharmaceutically acceptable salt, and/or in the form of a pharmaceutically acceptable hydrate. For example pharmaceutically acceptable salts of compounds of Formula I, include those derived from mineral acids such as for example: methane sulfonic acid, hydrochloric acid, nitric acid, phosphoric acid, sulfuric acid, hydrobromic acid, hydroiodic acid, nitrous acid, and phosphorous acid. Pharmaceutically acceptable salts may also be developed with organic acids including aliphatic mono and dicarboxylates and aromatic acids.

Other pharmaceutically acceptable salts of compounds of the present invention include for example sulfate, pyrosulfate, bisulfate, bisulfite, nitrate, and phosphate.

Compounds of Formula I can be made by processes known in the chemical arts for the production of structurally analogous compounds. Accordingly, the compounds of this invention may be prepared by employing procedures known in the literature starting from known compounds or readily prepared intermediates. For example, intermediate compounds designated as hydrochlorides also contain in most instances one equivalent of lithium chloride. For example, the core bicyclic, heterocyclic structure may be made by first preparing a chromone, quinolone or quinoline. The compounds of the present invention are made by the general procedure for amide coupling, that is, by coupling an anime with an acid using known coupling procedures. The amines used in the current invention if not commercially available may be made by known techniques. For example as a first step in the process of making compound of Formula I, a nitro compound may be reduced to an amine. The nitro compound may be a nitrophenyl compound. The resulting amines may be reacted with an acid.

Provided herein are synthetic methods for the preparation of precursor compounds or use in practicing aspects of the present invention.

It will be appreciated by those skilled in the art that certain compounds of the present invention contain for example asymmetrically substituted carbon, and accordingly may exist in and be isolated in, optically-active and racemic forms. Some compounds may exhibit polymorphism, thus it is to be understood that the present invention encompasses racemic, optically active, polymorphic or stereoisomeric forms, or mixtures thereof, which forms possess properties useful in the treatment of the disorders set forth below. Preparation of optically active forms is well known in the art (for example by resolution of racemic forms by recrystallization techniques, synthesis from optically active starting materials, chiral synthesis, or by chromatographic separation using a chiral stationary phase.

Compounds of Formula I have been found to be 5-HT_{1B} antagonists. The compounds of Formula I, and their pharmaceutically acceptable salts, may also be used in a method for the treatment of anxiety disorders, cognitive disorders, or mood disorders. The treatment of such disorders comprises administering to a warm-blooded animal, preferably a mammal, more preferably a human, in need of such treatment, an effective amount of a compound of Formula I or a pharmaceutically acceptable salt of said compound.

Further provided is the use of a compound of Formula I in the preparation of a medicament for the treatment of a disorder such as migraine in a warm-blooded animal, preferably a mammal, more preferably a human, suffering from such disorder.

The invention further provides a pharmaceutical composition suitable for the treatment of the above describe disorders comprising administering to a warm-blooded animal having such disorder an effective amount of a pharmaceutical composition of a compound of Formula I, or a pharmaceutically acceptable salt.

The invention also provides a pharmaceutical composition comprising a compound of Formula I, as defined herein, or a pharmaceutically acceptable salt, in combination with a pharmaceutically acceptable carrier. Preferred compounds of Formula I, for use in the compositions of the invention are as described above.

Compounds described herein demonstrate binding affinities (observed Ki values), in an assay described herein, of better than 10 μM. Selected compounds of the present invention are found to be active antagonists with activity of less than 100 μM/kg. In addition, selected compounds of the present invention demonstrate 5-HT_{1B} antagonist activity by reversing 5-HT_{1B} agonist-induced hypothermia in the guinea pig.

The compounds described herein may be provided or delivered in a form suitable for oral use, for example in a tablet, lozenge, hard and soft capsule, aqueous solution, oily solution, emulsion, and suspension. The compounds may be also be provided for topical administration, for example, as a cream, ointment, gel, spray, or aqueous solutions, oily solutions, emulsions or suspensions. The compounds described herein may also be provided in a form suitable for nasal administration for example, as a nasal spray, nasal drops, or dry powder. The compositions may also be administered to the vagina or rectum in the form of a suppository. The compounds described herein may also be administered parentally, for example by intravenous, intravesicular, subcutaneous, or intramuscular injection or infusion. The compounds may be administered by insufflation (for example as a finely divided powder). The compounds may also be administered transdermally or sublingually. The compounds of the invention may accordingly be obtained by conventional procedures using conventional pharmaceutical excipients, well known in the art. Thus, compositions intended for oral use may contain, for example, one or more coloring, sweetening, flavoring and/or preservative agents.

The amount of active ingredient that is combined with one or more excipients to produce a single dosage form will necessarily vary depending upon the host treated and the particular route of administration. The size of the dose for therapeutic or prophylactic purposes of a compound of the Formula I, will naturally vary according to the nature and severity of the conditions, the age and sex of the animal or patient and the route of administration, according to well known principles of medicine. Various assays and in vivo tests are known for determining the utility of the compounds in the disorders noted above and specifically as agonists and antagonists of 5-HT_{1B} and 5-HT_{1D} receptors.

A compound of formula (I) or a pharmaceutically acceptable salt, solvate or in vivo hydrolysable ester thereof, or a pharmaceutical composition or formulation comprising a compound of formula (I) is administered concurrently, simultaneously, sequentially or separately with another compound or compounds selected from the following:
(i) antidepressants such as amitriptyline, amoxapine, bupropion, citalopram, clomipramine, desipramine, doxepin duloxetine, elzasonan, escitalopram, fluvoxamine, fluoxetine, gepirone, imipramine, ipsapirone, maprotiline, nortriptyline, nefazodone, paroxetine, phenelzine, protriptyline, reboxetine, sertraline, sibutramine, thionisoxetine, tranylcypromaine, trazodone, trimipramine, venlafaxine and equivalents including but not limited to pharmaceutically acceptable salts and pharmaceutically active isomer(s) and metabolite(s) thereof.
(ii) atypical antipsychotics including for example quetiapine and lithium and equivalents including but not limited to pharmaceutically acceptable salts pharmaceutically and active isomer(s) and metabolite(s) thereof.
(iii) antipsychotics including for example amisulpride, aripiprazole, asenapine, benzisoxidil, bifeprunox, carbamazepine, clozapine, chlorpromazine, debenzapine, divalproex, duloxetine, eszopiclone, haloperidol, iloperidone, lamotrigine, loxapine, mesoridazine, olanzapine, paliperidone, perlapine, perphenazine, phenothiazine, phenylbutlypiperidine, pimozide, prochlorperazine, risperidone, sertindole, sulpiride, suproclone, suriclone, thioridazine, trifluoperazine, trimetozine, valproate, valproic acid, zopiclone, zotepine, ziprasidone and equivalents including but not limited to pharmaceutically acceptable salts and pharmaceutically active isomer(s) and metabolite(s) thereof.
(iv) anxiolytics including for example alnespirone, azapirones, benzodiazepines, barbiturates such as adinazolam, alprazolam, balezepam, bentazepam, bromazepam, brotizolam, buspirone, clonazepam, clorazepate, chlordiazepoxide, cyprazepam, diazepam, diphenhydramine, estazolam, fenobam, flunitrazepam, flurazepam, fosazepam, lorazepam, lormetazepam, meprobamate, midazolam, nitrazepam, oxazepam, prazepam, quazepam, reclazepam, tracazolate, trepipam, temazepam, triazolam, uldazepam, zolazepam and equivalents including but not limited to pharmaceutically acceptable salts and pharmaceutically active isomer(s) and metabolite(s) thereof.
(v) anticonvulsants including for example carbamazepine, topiramate, valproate, lamotrigine, gabapentin and equivalents including but not limited to pharmaceutically acceptable salts and pharmaceutically active isomer(s) and metabolite(s) thereof.
(vi) Alzheimer's therapies including for example donepezil, memantine, tacrine and equivalents including but not limited to pharmaceutically acceptable salts and pharmaceutically active isomer(s) and metabolite(s) thereof.
(vii) Parkinson's therapies including for example deprenyl, L-dopa, Requip, Mirapex, MAOB inhibitors such as selegine and rasagiline, comP inhibitors such as Tasmar, A-2 inhibitors, dopamine reuptake inhibitors, NMDA antagonists, Nicotine agonists, Dopamine agonists and inhibitors of neuronal nitric oxide synthase and equivalents including but not limited to pharmaceutically acceptable salts and pharmaceutically active isomer(s) and metabolite(s) thereof.
(ix) stroke therapies including for example abciximab, activase, (NXY-059), citicoline, crobenetine, desmoteplase, repinotan, traxoprodil and equivalents including but not limited to pharmaceutically acceptable salts and pharmaceutically active isomer(s) and metabolite(s) thereof.
(x) over active bladder/urinary incontinence therapies including for example darifenacin, falvoxate, oxybutynin, propiverine, robalzotan, solifenacin, tispium, tolterodine and equivalents including but not limited to pharmaceutically acceptable salts and pharmaceutically active isomer(s) and metabolite(s) thereof.
(xi) neuropathic pain therapies including for example gabapentin, lidoderm, pregablin and equivalents including but not limited to pharmaceutically acceptable salts and pharmaceutically active isomer(s) and metabolite(s) thereof.
(xii) nociceptive pain therapies such as celecoxib, etoricoxib, lumiracoxib, rofecoxib, valdecoxib, dictofenac, loxoprofen, naproxen, paracetamol and equivalents including but not limited to pharmaceutically acceptable salts and pharmaceutically active isomer(s) and metabolite(s) thereof.
(xiii) insomnia therapies including for example allobarbital, alonimid, amobarbital, benzoctamine, butabarbital, capuride, chloral, cloperidone, clorethate, dexclamol, eszopiclone, ethchlorvynol, etomidate, glutethimide, halazepam, hydroxyzine, mecloqualone, melatonin, mephobarbital, methaqualone, midaflur, nisobamate, pentobarbital, phenobarbital, propofol, roletamide, triclofos, secobarbital, zaleplon, zolpidem and equivalents including but not limited to pharmaceutically acceptable salts and pharmaceutically active isomer(s) and metabolite(s) thereof.
Such combination products employ the compounds of this invention within the dosage range described herein and the other pharmaceutically active agent within approved dosage ranges and/or the dosage described in the publication reference.

The utility of the compounds for example to treat anxiety disorders or mood disorders such as depression or dementia and other cognitive disorders such as Alzheimer's disease may be shown via a learned helplessness test in guinea pigs, which is used extensively as correlative to antidepressant activity in humans. The learned helplessness test may be carried out as follows: Seventy male Hartley guinea pigs, each weighing about 350-425 gm are fed ad lib, and are housed under a 12-hour light/dark cycle. The procedure consists of two phases: The induction phase and the avoidance training phase. In the induction phase, subjects are placed into standard shuttle cages (20 L×16 W×21 centimeters H ) which are fitted with a grid floor. Electrical stimulation (1.25 mA, 10 sec duration) is delivered to the floor of the cage every 90-sec during 1 hour daily sessions. Subjects have no opportunity to escape or to avoid shocks. Induction is conducted for 2 consecutive days.

In avoidance training, testing may be conducted in the shuttle cages, except that the subjects are not returned to the same chamber in which induction had occurred. Additionally, all cages are fitted with a partition with an arch in the center of the cage, through which animals can pass between the left and right halves of the cage. The procedure employed is a standard shuttle avoidance procedure in which a compound, conditioned stimulus (a 10-sec presentation of a tone and turning on of a lamp on the side of the cage that the guinea pig was occupying) serves to indicate presentation of electrical current to the floor of the cage. Shock is presented for a 5 sec period, 5 sec after initiation of the conditioned stimulus. Entry into the opposite side of the shuttle cage via the arched partition prior to shock onset results in the end of the trial (avoidance response). If shock is delivered, entry into the opposite side of the cage results in termination of the shock and CS (escape). Reversal of learned helplessness in the induction subjects correlates to antidepressant activity of the test compound. Avoidance training, 45-min in duration, is conducted on 2 consecutive days, beginning 48 hr after the final induction session. Seventy subjects are assigned to 1 of 6 groups of 11-12 animals. The groups are as follows:
1) No induction group. The subjects are placed into the shuttle cages but are not given inescapable shock, the animals are subsequently trained in the avoidance procedure and the vehicle is administered;

Induction vehicle control group;

Imipramine 17.8 mg/kg;

0.3 mg/kg compounds;

1 mg/kg compounds; and

5 mg/kg compounds.

Groups 2-6 are given induction and avoidance training sessions. Injections are administered immediately following induction sessions and 1 hour prior to avoidance training sessions. A second injection is administered 7-8 hours following the first injection, for a total of 9 injections administered over 5 days. No injections are administered following the final avoidance training session.

Compounds of the present invention may be administered in a volume of 1 mL/kg bwt. Imipramine is dissolved in DI water. The compounds are dissolved in DI water, to which was added a few drops of lactic acid (pH 5.5). The vehicle control is DI water prepared with lactic acid to the same pH as the-treated groups.

The primary dependent variable is escape failure during avoidance training. 2-way analysis of variance (ANOVA) is used to assess overall treatment effect, with Dunn's post hoc analysis used to compare the vehicle-treated group with the drug-treated groups. The no-induction group is used to gauge whether learned helplessness is established, by comparison to the vehicle treated group.

Other assays that may be used to measure for example affinity of compounds of the present invention for 5-HT_{1B} and 5-HT_{1D} receptors are described in J. Med. Chem. 41:1218-1235, 1228 (1998) and J. Med. Chem. 42:4981-5001, (1999) and incorporated by reference herein. These assays may be used with some modifications: Frozen membrane preparations of a stably transfected chinese hamster ovary (CHO) cell line expressing 5-HT_{1B} receptors and 5-HT_{1D} receptors may be thawed rapidly, briefly vortexed, and diluted in assay buffer (AB) containing 50 mM Tris-HCl, 4 mM MgCl₂, 4 mM CaCl₂, 1 mM EDTA, and adjusted to pH 7.4 with NaOH. Final protein concentrations are −0.185 mg/ml for 5-HT_{1B}, and 0.4 mg/ml for 5-HT_{1D} membranes. Test compounds are evaluated in competition assays using [³H]-GR125743 (Amersham). The ligand concentration in both assays was 0.27 nM. Kd for [³H]-GR125743 may vary from 0.15 nM to 0.25 nM. The 5-HT_{1B} and 5-HT_{1D} assays are performed simultaneously on one 96-well assay plate, one drug/compound per plate. Ten serial dilutions (1 uM to 4 pM, final concentration) of compound are prepared in DMSO from 10 mM stock solutions. Incubation mixtures are prepared in quadruplicate in 96-deep well assay plates (Matrix 1 ml). Final assay volumes per well are 10 μl compound/nonspecific; 100 μl membranes; 100 μl [3H]-GR125743; and 790 μl AB. Specific binding is defined by using 10 uM Methiothepine. The assay plates are shaken for 5 min., and then incubated for an additional 55 min. Then the assay plates are filtered through Beckman GF/B filters (soaked >2 hrs. in PEI) using a Packard Filtermate 196. Filters are washed 2× with 1 ml ice-cold wash buffer (5 mM Tris-HCl-pH7.4 with NaOH). After the filters are dried, 35 μl of Microscint20 is added to each well. The plates are then counted on a Packard TopCount to determine CPM's per well. Ki values are determined for each test compound utilizing the graphic and analytical software package, GraphPad Prism. Compounds are then ranked in order of potency, and selectivity for 5-HT_{1B} over 5-HT_{1D} receptors.

A method that may be used to determine a compound's affinity for 5-HT_{1B} and 5-HT_{1D} receptors is a guinea pig cortical test. This assay is described in detail by Roberts, et al, Br. J. Pharmacol. 1996, 117, 384-388. The test is carried out as follows: Guinea pigs are decapitated and the cortici is dissected out, weighed and homogenized in 50 mM Tris-HCl, pH 7.7 with an Ultra-Turrax followed by centrifugation for 10 min at 48000×g and 5° C. The pellet is resuspended and recentrifuged. The final pellet is suspended in 0.32 M sucrose buffer to a concentration of 0.5 g original wet weight per mL and stored frozen at −70° C. The radioligand binding assay is carried out as follows: [³H]GR125743 saturation studies are tested in duplicate with 3-4 mg w.w. per tube in 5 mL buffer (50 mM Tris, 4 mM CaCl2, 4 mM MgCl2 and 1 mM EDTA at pH 7.7), and a concentration range of 0.012-2 nM (10-12 concentrations) for the radioligand. Non-specific binding is determined in the presence of 10 mM methiothepin. In competition experiments 4-8 mg w.w. per tube and a radioligand concentration of 0.2 nM are used with 10-12 concentrations of the competing drug. The assays are run for 2-4 hours at 30° C. and terminated by rapid filtration through Whatman GF/B filters (pretreated with 0.1% polyethyleneimine) using a Brandel cell harvester. Bovine serum albumin (0.1%) is added to the washing buffer to reduce non-specific binding. Data from the experiments may be analyzed using the iterative non-linear curve-fitting program LIGAND. The K_{d} values obtained from the saturation studies are used in the calculation of the Ki values by the LIGAND program. The K_{d} value of [³H]GR125743 may result in a measurement of 46±4 pM and the Bₘₐₓ in a measurement of 4.9±0.2 pmol/g w.w.

A GTPγS binding assay may be used to determine whether a compound is a 5-HT_{1B} or 5-HT_{1D} agonist or antagonist. One assay available measures agonist stimulated GTP binding for example as set forth by Lazareno, S. (1999) Methods in Molecular Biology 106: 231-245. Frozen membranes may be thawed, briefly sonicated, and diluted to 167 μg/ml protein in assay buffer containing 20 mM HEPES, 100 mM NaCl, 1 mM MgCL₂ and 1 μM GDP, pH adjusted to 7.4 with NaOH. Diluted membranes are briefly homogenized with a Polytron and allowed to equilibrate at room temperature for at least 15 minutes before use. Serial dilutions (10 μM to 1 pM, final concentration) of test compounds are prepared in buffer with and without 100 nM 5-HT (final concentration) from 10 mM DMSO stock solutions. Incubation mixtures are prepared in quadruplicate in 96-well, deep-well plates and consisted of 180 μL of membranes (30 μg protein) and 40 μL of compound with or without 5-HT. After an incubation period of 15 minutes at room temperature, 20 μL of [³⁵S]GTPγS (NEN; 100 pM final concentration) is added to begin the assay. Mixtures are shaken for 2 minutes and incubated at room temperature for an additional 28 minutes. The reaction is stopped by rapid filtration through Beckman GF/B glass fiber filters using a 96-well Packard cell harvester. Filters are washed four times with 1 mL ice-cold water. The filter plates are nominally dried and 30 μL of scintillation cocktail (MicroScint 40, Packard) is added to each well. CPMs for each well is determined using a TopCount Scintillation Counter (Packard). Maximum stimulation of [³⁵S]GTPγS binding is defined in the presence of 100 nM 5-HT. Basal [³⁵S]GTPγS binding is defined in buffer alone. IC₅₀ values are defined as the concentration of compound at which 50% of the 100 nM 5-HT response was obtained. Maximal intrinsic activity (IA) of a compound is defined as the percent maximal 5-HT-induced stimulation by 10 μM compound in the absence of 5-HT. As an inter-assay standard, a concentration response curve of 5-HT (1 μM to 1 pM final) in the absence of compounds was included in each assay and an EC₅₀ was determined.

The following reference examples illustrate the making of intermediates in the synthesis of the compounds of the present invention, and are not intended to limit the invention in any manner.

The resolution of racemic compounds was achieved by a variety of methods including: resolution of racemic forms by recrystallization techniques, synthesis from optically active starting materials, chiral synthesis, HPLC chromatagraphic separation using a chiral stationary phase and supercritical fluid chromatography (SFC) using a chiral stationary phase.

Preparative HPLC was performed using either 21×250 mm columns (˜20 mL/min flow rates) for samples up to 300 mg or 50×500 mm (˜150 mL/min flow rates) for samples above 300 mg. A variety of chiral stationary phases (Chiralpak AD, 10 micron, for example) and a variety of solvent mixtures are used and are described in the following synthetic details. UV detection was either single or multi-wavelength set at 220, 254 and 280 nm.

Preparative SFC was achieved using a Berger auto prep 2 instrument using 21.2×250 mm columns. UV detection was 280 nm. A variety of chiral stationary phases (Chiralpak AD-H, 5 micron for example) and a variety of solvent mixtures are used and are described in the following synthetic details. Flow rate was 50 mL/min.

### REFERENCE EXAMPLE 9

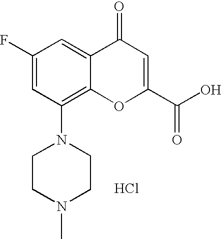

Preparation of 6-Fluoro-8-(4-methyl-piperazin-1-yl)-4-oxo-4-H-chromene-2-carboxylic acid hydrochloride. This material was prepared using the procedure described in AstraZeneca patent application WO2003037872.

### REFERENCE EXAMPLE 10

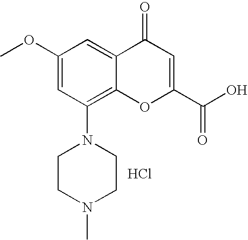

Preparation of 6-methoxy-8-(4-methyl-piperazin-1-yl)-4-oxo-4-H-chromene-2-carboxylic acid hydrochloride. This material was prepared using the procedure described in AstraZeneca patent application WO2003037872.

### REFERENCE EXAMPLE 11

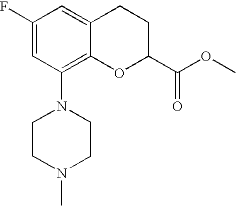

Resolution of (rac)-6-fluoro-8-(4-methyl-piperazin-1-yl)-chroman-2-carboxylic acid methyl ester to yield (+)-6-fluoro-8-(4-methyl-piperazin-1-yl)-chroman-2-carboxylic acid methyl ester

A solution of (rac)-6-fluoro-8-(4-methyl-piperazin-1-yl)-chroman-2-carboxylic acid methyl ester (Reference Example 17, 82.54 g) in methanol (300 mL) was treated with a solution of D-tartaric acid (42.24 g) in methanol (300 mL) and stirred rapidly for 16 h. The resulting solid was collected by filtration, redissolved in boiling methanol (600 mL), stirred for 16 h and the solid was filtered. This process was repeated for a total of 4 times to obtain the resolved tartrate salt of the title compound (30.29 g). A sample of this material was converted to the free base and analyzed by chiral HPLC (Chiralpak AD, 10% EtOH/Hexane, rt=7.75 min.) to determine the chiral purity of the material to be 95% ee. (The % ee enrichment varied slightly from batch to batch. Also, the ee could be further enriched by additional crystallizations.) A suspension of the resolved tartrate salt (30.29 g) in ethyl acetate (400 mL) was treated with 10% potassium carbonate solution and the organic layer was dried over anhydrous potassium carbonate, concentrated under reduced pressure to afford the title compound (20 g, 48% yield).

### REFERENCE EXAMPLE 12

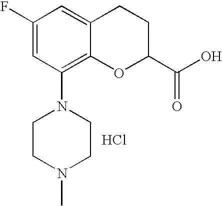

### Preparation of (+)-6-fluoro-8-(4-methyl-piperazin-1-yl)-chroman-2-carboxylic acid hydrochloride

(+)-6-fluoro-8-(4-methyl-piperazin-1-yl)-chroman-2-carboxylic acid methyl ester (Reference Example 11, 20.0 g) was dissolved in tetrahydrofuran (500 mL), treated with a solution of lithium hydroxide (3.12 g) in water (100 mL). Upon stirring for 1 h at room temperature the reaction mixture was acidified with 4M HCl in dioxane (33 mL) and concentrated under reduced pressure. The resulting oil was treated consecutively twice with tetrahydrofuran and once with acetonitrile (300 mL) and concentrated under reduced pressure. The material thus obtained was suspended in rapidly stirred acetonitrile (400 mL), treated with 4M HCl in dioxane (65 mL) and stirred for 1 h. The solid was filtered, resuspended in ethyl ether (300 mL) and filtered, dried under reduced pressure to obtain the desired product (25.7 g)

LC/MS: 295 (M+1). ¹H NMR (300.132 MHz, DMSO), δ 11.25 (s, 2H), 6.64-6.55 (m, 2H), 4.83 (dd, J=6.3, 4.0 Hz, 1H), 3.92 (d, J=12.8 Hz, 1H), 3.45-3.35 (m, 3H), 3.22-3.11 (m, 3H), 2.92 (t, J=11.3 Hz, 1H), 2.83-2.73 (m, 4H), 2.68-2.57 (m, 2H), 2.19-1.99 (m, 2H).

### REFERENCE EXAMPLE 13

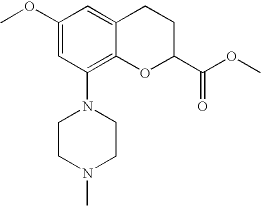

### Racemization of (−)-6-methoxy-8-(4-methyl-piperazin-1-yl)-chroman-2-carboxylic acid methyl ester to yield (rac)-6-methoxy-8-(4-methyl-piperazin-1-yl)-chroman-2-carboxylic acid methyl ester

To a solution containing (−)-6-methoxy-8-(4-methyl-piperazin-1-yl)-chroman-2-carboxylic acid methyl ester (Reference Example 18, 93.1 mmol) dissolved in anhydrous methanol (500 mL) under nitrogen atmosphere was added sodium methoxide (25% wt in MeOH, 106.7 mmol). The resulting solution was refluxed for 24 hours. The reaction was cooled to room temperature. Dropwise, concentrated sulfuric acid (162 mmol) was added and the solution was refluxed for 90 minutes. Reaction was cooled to room temperature. Solvent was removed under vacuum. Residue was suspended in ethyl acetate (500 ML). Dropwise, saturated sodium bicarbonate was added until pH was basic by litmus. The basic aqueous layer was extracted with additional ethyl acetate (2×400 mL). The combined ethyl acetate extracts are washed with saturated sodium bicarbonate (250 mL) then brine (300 mL). The organic phase was dried (MgSO₄), filtered and concentrated yielding an orange solid. Solid was washed with a solution containing ether (50 mL) and hexane (400 mL) then with hexane (50 mL) yielding a pale yellow solid (27 g, 91% yield). LC/MS (M+1), m/z: 321. ¹H-NMR (300 MHz, CDCl₃), δ 6.39 (d, 1H, J=3 Hz), 6.21 (d, 1H, J=3 Hz), 4.79 (dd, 1H, J₁=7.2 Hz, J₂=3.6 Hz), 3.77 (s, 3H), 3.73 (s, 3H), 3.38 (bm, 2H), 2.93 (bm, 2H), 2.74 (m, 2H), 2.63 (bm, 4H), 2.36 (s, 3H), 2.28 (m, 1H), 2.17 (m, 1H). Chiral HPLC (Chiralpak AD, 20% IPA/Hexane) RT₁=5.03 min, 49.6% (isomer 1), RT₂=6.98 min, 48.9% (isomer 2).

This method can also be used to racemize any of the other isomers described including: (+)-6-methoxy-8-(4-methyl-piperazin-1-yl)-chroman-2-carboxylic acid methyl ester,

(−)-6-fluoro-8-(4-methyl-piperazin-1-yl)-chroman-2-carboxylic acid methyl ester and (+)-6-fluoro-8-(4-methyl-piperazin-1-yl)-chroman-2-carboxylic acid methyl ester.

### REFERENCE EXAMPLE 14

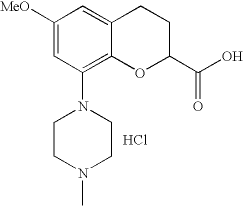

### Preparation of (Rac)-6-methoxy-8-(4-methyl-piperazin-1-yl)-chroman-2-carboxylic acid hydrochloride

A solution of 6-methoxy-8-(4-methyl-piperazin-1-yl)-4-oxo-4-H-chromene-2-carboxylic acid hydrochloride (Reference Example 10, 100 g) in acetic acid (1200 mL) was hydrogenated using 10% Pd/C (25 g) at 50 PSI hydrogen pressure at 60 C for 5 days. At the end of this period the reaction mixture was filtered through a pad of diatomaceous earth (washed with acetic acid [6×150 mL]) and concentrated under reduced pressure yielding a thick amber syrup. The diatomaceous earth pad was then washed with THF (5×150 mL). These washes are combined with the amber syrup and solvent removed under vacuo. Anhydrous toluene (4×500 mL) was added and the solution was again concentrated in vacuo. The syrup thus obtained was stirred with ethyl ether (1000 mL) for 1 h and then stored undisturbed overnight. The solid was collected by vacuum filtration, washed with ether (2×200 mL) and dried under vacuum at 50 C to yield a light beige powder (185.4 g). This beige powder was suspended in rapidly stirred acetonitrile (2500 mL). To this rapidly stirring suspension was added 4N HCl in dioxane (560 mL) dropwise over 15 minutes, and the resulting suspension stirred overnight. The off-white solid was collected by filtration, washed with acetonitrile (3×200 mL), dried under vacuum at 55 C overnight yielding the title compound (167.8 g, 87% yield as the mono-HCl salt). LC/MS: 307 (M+1).

### REFERENCE EXAMPLE 15

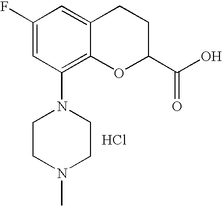

### Preparation of (Rac)-6-Fluoro-8-(4-methyl-piperazin-1-yl)-chroman-2-carboxylic acid hydrochloride

This material was prepared as described in Reference Example 14 but using 6-Fluoro-8-(4-methyl-piperazin-1-yl)-4-oxo-4-H-chromene-2-carboxylic acid hydrochloride (Reference Example 9, 150 g) instead of 6-methoxy-8-(4-methyl-piperazin-1-yl)-4-oxo-4-H-chromene-2-carboxylic acid hydrochloride and the reaction temperature was raised to 70 C yielding the title compound (141 g, 87% yield as the mono-HCl salt). LC/MS: 295 (M+1).

### REFERENCE EXAMPLE 16

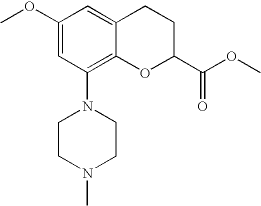

### Preparation of (Rac)-6-methoxy-8-(4-methyl-piperazin-1-yl)-chroman-2-carboxylic methyl ester

Concentrated sulfuric acid (1.0 mL) was added to a solution containing (Rac)-6-methoxy-8-(4-methyl-piperazin-1-yl)-chroman-2-carboxylic acid hydrochloride (Reference Example 14, 8.49 g) in anhydrous methanol (80 mL) and heated to reflux for 3 h. Reaction was cooled to room temp and solvent was removed under vacuo. Enough saturated aqueous sodium bicarbonate was added to give pH>8. Transfer to separatory funnel and extract with ethyl acetate (2×200 mL). The ethyl acetate extracts are washed with saturated sodium bicarbonate (75 mL) then brine (75 ml), dried over magnesium sulfate and filtered. Solvent was removed under vacuum yielding an orange solid. Solid was washed with hexane (75 mL) and dried yielding the title compound as an off-white solid (7.02 g, 99% yield). LC/MS: 321 (M+1).

### REFERENCE EXAMPLE 17

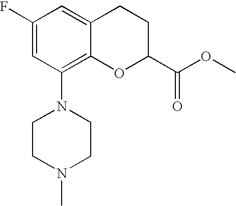

### Preparation of (Rac)-6-Fluoro-8-(4-methyl-piperazin-1-yl)-chroman-2-carboxylic acid methyl ester

This material was prepared as described in Reference Example 16 but using (Rac)-6-fluoro-8-(4-methyl-piperazin-1-yl)-chroman-2-carboxylic acid hydrochloride (Reference Example 15) instead of (Rac)-6-Methoxy-8-(4-methyl-piperazin-1-yl)-chroman-2-carboxylic acid hydrochloride yielding the title compound as an off-white solid. LC/MS: 309 (M+1).

### REFERENCE EXAMPLE 18

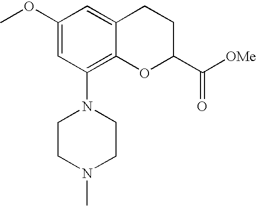

### Resolution of (rac)-6-methoxy-8-(4-methyl-piperazin-1-yl)-chroman-2-carboxylic acid methyl ester to yield (+)-6-methoxy-8-(4-methyl-piperazin-1-yl)-chroman-2-carboxylic acid methyl ester and (−)-6-methoxy-8-(4-methyl-piperazin-1-yl)-chroman-2-carboxylic acid methyl ester

(rac)-6-methoxy-8-(4-methyl-piperazin-1-yl)-chroman-2-carboxylic acid methyl ester (Reference Example 16) was resolved using preparative chiral HPLC (Chiralpak AD column [20μ, 5 cm×50 cm], 15% absolute ethanol in hexane, flow rate of 150 ml/min). Approximately 650 mg of (rac)-6-methoxy-8-(4-methyl-piperazin-1-yl)-chroman-2-carboxylic acid methyl ester (Reference Example 16) could be resolved per run.
Isomer 1 (−)-6-methoxy-8-(4-methyl-piperazin-1-yl)-chroman-2-carboxylic acid methyl ester

LC/MS: (M+1), m/z=321. ¹H NMR (300 MHz, CDCl₃), δ 6.39 (d, J=3.0 Hz, 1H), 6.21 (d, J=2.7 Hz, 1H), 4.78 (dd, J=7.2, 3.6 Hz, 1H), 3.78 (s, 3H), 3.73 (s, 3H), 3.38 (bm, 2H), 2.92 (bm, 2H), 2.73 (m, 2H), 2.63 (bm, 4H), 2.36 (s, 3H), 2.27 (m, 1H), 2.19 (m, 1H). Chiral HPLC (Chiralpak AD, 20% IPA/Hexane) RT=3.63 min, 98.6%. [α]_{D}²⁵=−95.8, (c 1, CCl₄).
Isomer 2 (+)-6-methoxy-8-(4-methyl-piperazin-1-yl)-chroman-2-carboxylic acid methyl ester

LC/MS: (M+1), m/z=321. ¹H NMR (300 MHz, CDCl₃), δ 6.39 (d, J=3.0 Hz, 1H), 6.21 (d, J=2.7 Hz, 1H), 4.78 (dd, J=7.6, 3.6 Hz, 1H), 3.78 (s, 3H), 3.73 (s, 3H), 3.38 (bm, 2H), 2.92 (bm, 2H), 2.73 (m, 2H) 2.63 (bm, 4H), 2.36 (s, 3H), 2.27 (m, 1H), 2.19 (m, 1H). Chiral HPLC (Chiralpak AD, 20% IPA/Hexane), RT=7.24 min, 99.6%. [α]_{D}²⁵=+96.3 (c 1, CCl₄).

### REFERENCE EXAMPLE 19

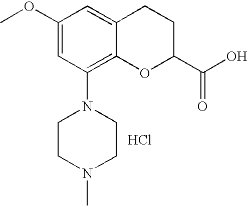

### Preparation of (+)-6-methoxy-8-(4-methyl-piperazin-1-yl)-chroman-2-carboxylic acid hydrochloride

This material was prepared as described in Reference Example 12 but using (+)-6-methoxy-8-(4-methyl-piperazin-1-yl)-chroman-2-carboxylic acid methyl ester (Reference Example 18, 10 g) instead of (+)-6-fluoro-8-(4-methyl-piperazin-1-yl)-chroman-2-carboxylic acid methyl ester yielding the title compound as an off-white solid (12.3 g, 100%).

LC/MS: 307 (M+1). ¹H NMR (300 MHz, CD₃OD), δ 6.47 (d, J=4.0 Hz, 1H), 6.41 (d, J=3.7 Hz, 1H), 4.02 (br s, 1H), 3.72 (s, 3H), 3.57 (br s, 3H), 3.4-3.2 (br m, 5H), 2.96 (s, 3H), 2.79 (m, 2H), 2.25 (m, 1H), 2.16 (m, 1H). [α]_{D}²⁵=+58 (c 1, MeOH).

### REFERENCE EXAMPLE 20

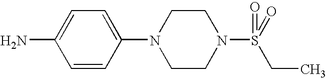

### Preparation of 4-(4-ethanesulfonyl-piperazin-1-yl)-phenylamine

### REFERENCE EXAMPLE 20A

### 4-(4-Nitrophenyl)-1-ethylsulfonylpiperazine

1-(4-Nitrophenyl) piperazine (100 g, 0.48 mol) was dissolved in dichloromethane (500 ml). Triethylamine (1.02 eq., 68 mL, 0.49 mol) was added and the reaction was cooled to 5 C. Ethanesulfonyl chloride (1.02 eq, 46.5 mL, 0.49 mol) was added dropwise over 1 hour keeping the temperature under 20 C. Allow reaction to stir and warm to room temperature over 3 hours. The solution was poured into saturated sodium bicarbonate and then extracted (3×) with dichloromethane, dried (MgSO₄), filtered and concentrated in vacuo to give 4-(4-nitrophenyl)-1-ethanesulfonylpiperazine as a yellow solid (143 g, quantitative yield).

GC/MS: (EI, M+), m/z=300.

### REFERENCE EXAMPLE 20B

### 4-(4-ethanesulfonyl-piperazin-1-yl)-phenylamine

4-(4-Nitrophenyl)-1-ethylsulfonylpiperazine (5.0 g, 16.7 mmol), as prepared above, was mixed in ethanol (100 ml) and 10% palladium on carbon (500 mg) was added. The mixture was hydrogenated on a Parr apparatus (50 psi) for 6 h. The catalyst was filtered, washed with ethanol and concentrated in vacuo to give 4-[4-(ethylsulfonyl)-1-piperazinyl]phenylamine as a light purple solid. This produce was used immediately.

### REFERENCE EXAMPLE 35

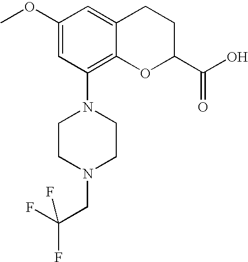

### Preparation of 6-methoxy-8-[4-(2,2,2-trifluoro-ethyl)-piperazin-1-yl]-chroman-2-carboxylic acid

### REFERENCE EXAMPLE 35A

### 6-methoxy-4-oxo-8-[4-(2,2,2-trifluoro-ethyl)-piperazin-1-yl]-4H-chromene-2-carboxylic acid ethyl ester

To a reaction flask was added 1-(2,2,2-trifluoro-ethyl)-piperazine (Reference Example 36, 284 mg, 1.69 mmol), 8-bromo-6-methoxy-4-oxo-4H-chromene-2-carboxylic acid ethyl ester (Reference Example 37, 570 mg, 1.74 mmol), toluene (10 mL), racemic-2,2′-bis(diphenylphosphino)-1,1′-binaphthyl (100 mg, 0.16 mmol), cesium carbonate (880 mg, 2.70 mmol), and palladium dba (70 mg, 0.076 mmol). Flask was evacuated and backfilled with N₂ (3 cycles). Reaction was heated to 100° C. for 18 h, cooled to rt, mixed with 50 mL 20% aqueous K₂CO₃, and extracted with DCM (4×30 mL). Extracts are combined, dried over Na₂SO₄, filtered, and evaporated under reduced pressure to give an oil. Product was purified by chromatography on silica, 200:1 to 100:1 to 50:1 to 25:1 DCM/Hexane:MeOH (w/0.25% aq conc. NH₃). This gave 319 mg of product (46%), MS: m/z 415 (M+H).

### REFERENCE EXAMPLE 35B

### 6-methoxy-4-oxo-8-[4-(2,2,2-trifluoro-ethyl)-piperazin-1-yl]-4H-chromene-2-carboxylic acid

To a solution of 6-methoxy-4-oxo-8-[4-(2,2,2-trifluoro-ethyl)-piperazin-1-yl]-4H-chromene-2-carboxylic acid ethyl ester (Reference Example 35a, 319 mg, 0.77 mmoles) in 6 mL of 5:1 THF:Methanol was added LiOH (62 mg, 1.48 mmol) dissolved in 1 mL Water. After 15 minutes the pH was then adjusted to ˜1 and solvents are evaporated under reduced pressure to give a yellow solid that was pumped down overnight. This gave 320 mg of product (89% as the HCl/LiCl salt). MS: m/z 387 (M+H).

### REFERENCE EXAMPLE 35C

### 6-methoxy-8-[4-(2,2,2-trifluoro-ethyl)-piperazin-1-yl]-chroman-2-carboxylic acid

6-methoxy-4-oxo-8-[4-(2,2,2-trifluoro-ethyl)-piperazin-1-yl]-4H-chromene-2-carboxylic acid salt with HCl/LiCl (Reference Example 35b, 320 mg, 0.69 mmol) was dissolved in 250 mL of HOAc and charged with 240 mg 10% Pd/C. Flask was attached to the Parr shaker, evacuated/backfilled with hydrogen (3 cycles). Mixture was heated to 75° C. and agitated for 8 h under 60-psi hydrogen. Reaction was cooled to RT, filtered through diatomaceous earth, and evaporated under reduced pressure. The resulting brown oil was pumped down under high vacuum overnight. This gave 196 mg (63%), MS: m/z 375 (M+H).

### REFERENCE EXAMPLE 36

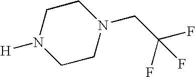

Preparation of 1-(2,2,2-trifluoro-ethyl)-piperazine. This material was prepared as described in JOC, 1966, 31, 3867-3868.

### REFERENCE EXAMPLE 37

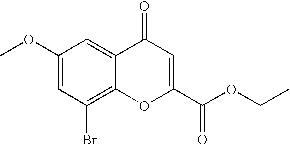

Preparation of 8-bromo-6-methoxy-4-oxo-4H-chromene-2-carboxylic acid ethyl ester. This material was prepared as described in AstraZeneca patent application WO2003037872.

### REFERENCE EXAMPLE 38

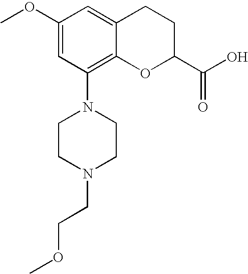

### Preparation of 6-methoxy-8-[4-(2-methoxy-ethyl)-piperazin-1-yl]-chroman-2-carboxylic acid

### REFERENCE EXAMPLE 38A

### 6-methoxy-8-[4-(2-methoxy-ethyl)-piperazin-1-yl]-4-oxo-4H-chromene-2-carboxylic acid ethyl ester

8-Bromo-6-methoxy-4-oxo-4H-chromene-2-carboxylic acid ethyl ester (Reference Example 37, 680 mg, 2.08 mmol) was coupled with 1-(2-methoxy-ethyl)-piperazine (320 mg, 2.22 mmol) in an analogous fashion to that described in Reference Example 35a to give 292 mg (36%) of product; MS: m/z 391 (M+H).

### REFERENCE EXAMPLE 38B

### 6-methoxy-8-[4-(2-methoxy-ethyl)-piperazin-1-yl]-4-oxo-4H-chromene-2-carboxylic acid

6-methoxy-8-[4-(2-methoxy-ethyl)-piperazin-1-yl]-4-oxo-4H-chromene-2-carboxylic acid ethyl ester (Reference Example 38a) was hydrolyzed using a procedure similar (reaction time was 30 minutes) to that described in Reference Example 35b to give 292 mg of product (essentially quantitative as the HCl/LiCl salt); MS: m/z 363 (M+H).

### REFERENCE EXAMPLE 38C

### 6-methoxy-8-[4-(2-methoxy-ethyl)-piperazin-1-yl]-chroman-2-carboxylic acid

6-methoxy-8-[4-(2-methoxy-ethyl)-piperazin-1-yl]-4-oxo-4H-chromene-2-carboxylic acid (Reference Example 38b) was reduced as described in Reference Example 35c except the reaction time was 50 h. This gave 310 mg of product (essentially quantitative). MS: m/z 351 (M+H).

### REFERENCE EXAMPLE 40

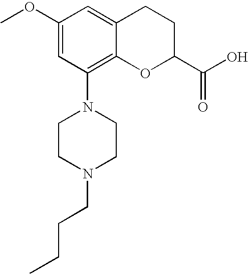

### Preparation of 8-(4-Butyl-piperazin-1-yl)-6-methoxy-chroman-2-carboxylic acid

### REFERENCE EXAMPLE 40A

### 8-(4-Butyl-piperazin-1-yl)-6-methoxy-4-oxo-4H-chromene-2-carboxylic acid ethyl ester

8-Bromo-6-methoxy-4-oxo-4H-chromene-2-carboxylic acid ethyl ester (Reference Example 37, 654 mg, 2.00 mmol) was coupled with 1-butyl-piperazine (360 mg, 2.53 mmol) in an analogous fashion to that described in Reference Example 35a. Resulting compound needed additional purification by Prep RPHPLC. [Conditions: 25-90% MeCN (w/0.1% TFA) over 20 minutes, 7 min equilibration at 25% MeCN, 5 min flush at 90% MeCN. Used a 2″ diameter radial compression C8 dynamax 60 Å column]. Residue after evaporation was mixed with 15 mL 20% aqueous K₂CO₃ and extracted with DCM (3×30 mL). Extracts are combined, dried over Na₂SO₄, filtered, and evaporated under reduced pressure to give 307 mg (41%) of product; ¹H NMR (300.132 MHz, CDCl3), δ 7.16 (d, J=2.9 Hz, 1H), 7.09 (s, 1H), 6.83 (d, J=2.9 Hz, 1H), 4.45 (q, J=7.1 Hz, 2H), 3.88 (s, 3H), 3.35-3.20 (m, 4H), 2.79-2.66 (m, 4H), 2.44 (t, J=7.6 Hz, 2H), 1.44 (t, J=7.1 Hz, 3H), 1.60-1.30 (m, 4H), 0.95 (t, J=7.2 Hz, 3H).

### REFERENCE EXAMPLE 40B

### 8-(4-Butyl-piperazin-1-yl)-6-methoxy-chroman-2-carboxylic acid

8-(4-Butyl-piperazin-1-yl)-6-methoxy-4-oxo-4H-chromene-2-carboxylic acid ethyl ester (Reference Example 40a, 307 mg, 0.79 mmol) was dissolved in 200 mL of HOAc and charged with 300 mg 10% Pd/C. Flask was attached to the Parr shaker and evacuated/backfilled with hydrogen (3 cycles). Mixture was heated to 75° C. and agitated for 5 days under 60-psi hydrogen. Reaction was cooled to room temperature, filtered through diatomaceous earth, and evaporated under reduced pressure. The resulting brown oil was triturated in diethyl ether, filtered, washed with more diethyl ether, and pumped down under high vacuum overnight. This gave 151 mg (55%), MS: m/z 349 (M+H).

### REFERENCE EXAMPLE 41

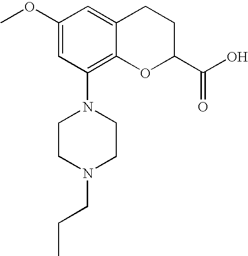

### Preparation of 8-(4-Propyl-piperazin-1-yl)-6-methoxy-chroman-2-carboxylic acid

### REFERENCE EXAMPLE 41A

### 6-methoxy-4-oxo-8-(4-propyl-piperazin-1-yl)-4H-chromene-2-carboxylic acid ethyl ester

8-Bromo-6-methoxy-4-oxo-4H-chromene-2-carboxylic acid ethyl ester (Reference Example 37, 701 mg, 2.14 mmol) was coupled with 1-propyl-piperazine (300 mg, 2.34 mmol) in an analogous fashion to that described in Reference Example 40a to give 312 mg (40%) of product; ¹H NMR (300.132 MHz, CDCl3), δ 7.16 (d, J=2.9 Hz, 1H), 7.09 (s, 1H), 6.83 (d, J=3.4 Hz, 1H), 4.45 (q, J=7.1 Hz, 2H), 3.88 (s, 3H), 3.35-3.20 (m, 4H), 2.80-2.65 (m, 4H), 2.42 (t, J=7.5 Hz, 2H), 1.66-1.49 (m, 2H), 1.44 (t, J=7.1 Hz, 3H), 0.95 (t, J=7.3 Hz, 3H).

### REFERENCE EXAMPLE 41B

### 6-methoxy-8-(4-propyl-piperazin-1-yl)-chroman-2-carboxylic acid

6-methoxy-4-oxo-8-(4-propyl-piperazin-1-yl)-4H-chromene-2-carboxylic acid ethyl ester (Reference Example 41a) was reduced as described in Reference Example 40b. This gave 104 mg of product (36%), MS: m/z 335 (M+H).

### REFERENCE EXAMPLE 42

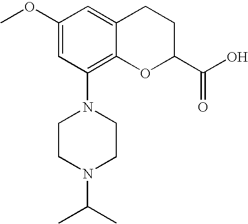

### Preparation of 8-(4-Isopropyl-piperazin-1-yl)-6-methoxy-chroman-2-carboxylic acid

### REFERENCE EXAMPLE 42A

### 6-methoxy-4-oxo-8-(4-isopropyl-piperazin-1-yl)-4H-chromene-2-carboxylic acid ethyl ester

8-Bromo-6-methoxy-4-oxo-4H-chromene-2-carboxylic acid ethyl ester (Reference Example 37, 696 mg, 2.13 mmol) was coupled with 1-isopropyl-piperazine (273 mg, 2.13 mmol) in an analogous fashion to that described in Reference Example 40a to give 380 mg of product (48%); ¹H NMR (300.132 MHz, CDCl3), δ 7.16 (d, J=2.9 Hz, 1H), 7.10 (s, 1H), 6.83 (d, J=2.9 Hz, 1H), 4.45 (q, J=7.1 Hz, 2H), 3.88 (s, 3H), 3.35-3.20 (m, 4H), 2.88-2.70 (m, 5H), 1.45 (t, J=7.1 Hz, 3H), 1.12 (d, J=6.5 Hz, 6H).

### REFERENCE EXAMPLE 42B

### 6-methoxy-8-(4-isopropyl-piperazin-1-yl)-chroman-2-carboxylic acid

6-methoxy-4-oxo-8-(4-isopropyl-piperazin-1-yl)-4H-chromene-2-carboxylic acid ethyl ester (Reference Example 42a) was reduced as described in Reference Example 40b. This gave 242 mg of product (72%); MS: m/z 334 (M+H).

### REFERENCE EXAMPLE 43

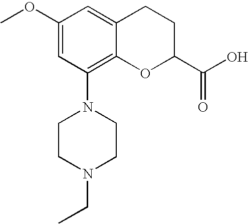

### Preparation of 8-(4-Ethyl-piperazin-1-yl)-6-methoxy-chroman-2-carboxylic acid

### REFERENCE EXAMPLE 43A

### 6-methoxy-4-oxo-8-(4-ethyl-piperazin-1-yl)-4H-chromene-2-carboxylic acid ethyl ester

8-Bromo-6-methoxy-4-oxo-4H-chromene-2-carboxylic acid ethyl ester (Reference Example 37, 690 mg, 2.11 mmol) was coupled with 1-ethyl-piperazine (243 mg, 2.13 mmol) in an analogous fashion to that described in Reference Example 40a to give 270 mg of product (35%); ¹H NMR (300.132 MHz, CDCl3), δ 7.16 (d, J=2.9 Hz, 1H), 7.10 (s, 1H), 6.83 (d, J=2.9 Hz, 1H), 4.45 (q, J=7.1 Hz, 2H), 3.88 (s, 3H), 3.36-3.22 (m, 4H), 2.82-2.66 (m, 4H), 2.53 (q, J=7.1 Hz, 2H), 1.44 (t, J=7.1 Hz, 3H), 1.15 (t, J=7.2 Hz, 3H).

### REFERENCE EXAMPLE 43B

### 6-methoxy-8-(4-ethyl-piperazin-1-yl)-chroman-2-carboxylic acid

6-methoxy-4-oxo-8-(4-ethyl-piperazin-1-yl)-4H-chromene-2-carboxylic acid ethyl ester Reference Example 43a) was reduced as described in Reference Example 40b. This gave 231 mg of product (72%); MS: m/z 321 (M+H).

### REFERENCE EXAMPLE 44

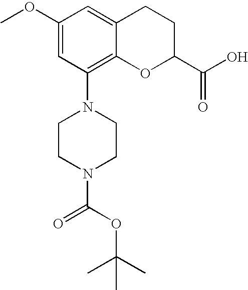

### Preparation of 4-(2-Carboxy-6-methoxy-chroman-8-yl)-piperazine-1-carboxylic acid tert-butyl ester

### REFERENCE EXAMPLE 44A

### 8-(4-Benzyl-piperazin-1-yl)-6-methoxy-4-oxo-4H-chromene-2-carboxylic acid ethyl ester

8-Bromo-6-methoxy-4-oxo-4H-chromene-2-carboxylic acid ethyl ester (Reference Example 37, 1.34 g, 4.10 mmol) was coupled with 1-benzyl-piperazine (1.34 g, 4.26 mmol) in an analogous fashion to that described in Reference Example 40a to give 1.21 g of product (70%). ¹H NMR (300.132 MHz, CDCl3), δ 7.42-7.22 (m, 5H), 7.16 (d, J=2.9 Hz, 1H), 7.09 (s, 1H), 6.82 (d, J=2.9 Hz, 1H), 4.44 (q, J=7.1 Hz, 2H), 3.87 (s, 3H), 3.62 (s, 2H), 3.35-3.18 (m, 4H), 2.83-2.65 (m, 4H), 1.43 (t, J=7.1 Hz, 3H).

### REFERENCE EXAMPLE 44B

### 6-methoxy-8-piperazin-1-yl-chroman-2-carboxylic acid

8-(4-Benzyl-piperazin-1-yl)-6-methoxy-4-oxo-4H-chromene-2-carboxylic acid ethyl ester (Reference Example 44A) was reduced as described in Reference Example 40B. Exception: reaction product was purified by RPHPLC as described in Reference Example 40A. This gave 225 mg of product (34%), MS: m/z 293 (M+H).

### REFERENCE EXAMPLE 44C

### 4-(2-Carboxy-6-methoxy-chroman-8-yl)-piperazine-1-carboxylic acid tert-butyl ester

To a stirred solution of 6-methoxy-8-piperazin-1-yl-chroman-2-carboxylic acid (Reference Example 44B, 297 mg, 1.23 mmol) and Et₃N (320 μl, 2.30 mmol) in 10 mL of DCM was added di-tert-butyl dicarbonate (272 mg, 1.24 mmol). Mixture was stirred for 18 h, diluted with 1 N HCl (10 mL), and extracted with DCM (3×20 mL). Organic layers are dried over Na₂SO₄, filtered, and solvent was evaporated under reduced pressure. Product was purified by chromatography on silica (DCM:MeOH 20:1 [symbol] 10:1) to give 190 mg of product.

MS: m/z 393 (M+H).

### REFERENCE EXAMPLE 47

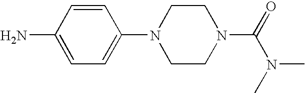

### Preparation of 4-(4-Amino-phenyl)-piperazine-1-carboxylic acid dimethylamide

A solution of 4-fluoronitrobenzene (2.7 mL, 25 mM) in EtOH was treated with piperazine (4.7 g, 55 mM) in one portion. The reaction was heated to reflux and stirred for 1 h. After cooling, the product is filtered and collected as a yellow solid. The solid was suspended in DCM and treated with DIPEA (1.7 mL, 9.4 mM) followed by neat N,N-dimethylcarbamoylchloride (0.71 mL, 7.9 mM). The reaction was stirred at reflux for 1 h. The reaction was quenched with MeOH and the solvent removed at reduced pressure. The product was recrystallized from EtOH. This solid was suspended in EtOH, treated with 10% Pd/C (0.2 g) and the reaction hydrogenated on a Parr Shaker at 50 psi for 2h. The reaction was then filtered and the solvent removed at reduced pressure to give the product as a gray solid (0.8 g, 13%). LC/MS: 249 (M+H, 100%); ¹H-NMR (300 MHz, DMSO-d₆), δ 7.03 (d, J=9.0 Hz, 2H), 6.60 (d, J=9.0 Hz, 2H), 3.75 (m, 4H), 3.72 (bs, 2H), 3.24 (m, 4H), 2.83 (s, 3H), 2.73 (s, 3H).

### EXAMPLES

### Example 2

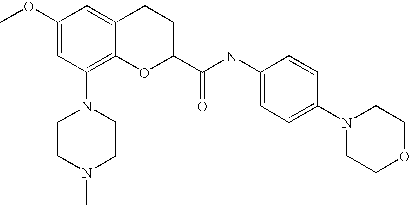

### (−)-6-methoxy-8-(4-methyl-piperazin-1-yl)-N-(4-morpholin-4-yl-phenyl)chroman-2-carboxamide

(rac)-6-methoxy-8-(4-methyl-piperazin-1-yl) chroman-2-carboxylic acid hydrochloride (Reference Example 14, 4.04 mmol) was suspended in anhydrous N,N-dimethylformamide (20 ml) with 4-morpholinoaniline (0.86 g, 4.84 mmol), and treated simultaneously with TBTU (2.60 g, 8.10 mmol) and HOBt (1.20 g, 8.88 mmol). Then N,N-diisopropylethylamine (2.82 ml, 16.2 mmol) was added, and the reaction was stirred 3 h at room temperature. The reaction mixture was poured into water (200 mL) and extracted with methylene chloride (2×150 mL). The combined organic phases are washed with water (3×100 mL), saturated sodium bicarbonate (100 mL), and saturated brine (100 mL), then dried (Na₂SO₄), filtered, and concentrated in vacuo to give the product (1.56 g). Trituration from 1:10:10 methylene chloride/ether/hexane gave 1.12 g lavendar solid. The racemic mixture was subjected to chiral HPLC resolution (Chiralpak AD, 25% ethanol/hexane), and following trituration from 4:1 ether/hexane 0.238 g (13%) (−)-6-methoxy-8-(4-methyl-piperazin-1-yl)-N-(4-morpholin-4-yl-phenyl)chroman-2-carboxamide was obtained.

(−): LC/MS (M+1), m/z: 467. ¹H NMR (300 MHz, CDCl₃), δ 8.68 (s, 1H), 7.49 (d, J=9.0 Hz, 2H), 6.90 (d, J=9.0 Hz, 2H), 6.44 (d, J=2.8 Hz, 1H), 6.30 (d, J=2.8 Hz, 1H), 4.66 (dd, J=10.0, 3.7 Hz, 1H), 3.86 (dd, J=4.8, 4.8 Hz, 4H), 3.75 (s, 3H), 3.37-3.22 (m, 2H), 3.13 (dd, J=4.8, 4.8 Hz, 4H), 3.04-2.83 (m, 3H), 2.77 (dt, J=16.1, 4.7 Hz, 1H), 2.71-2.50 (m, 5H), 2.36 (s, 3H), 2.01 (ddd, J=23.9, 10.3, 5.3 Hz, 1H). Chiral HPLC (Chiralpak AD, 25% EtOH/Hexane), RT=27.30 min, >99.0%.

### Example 17

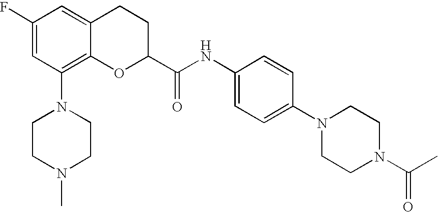 [0524] (+)-N-[4-(4-acetylpiperazin-1-yl)phenyl]-6-fluoro-8-(4-methylpiperazin-1-yl)chroman-2-carboxamide (isomer 1).

The title compound was prepared in 87% yield in the same manner as Example 2, using (+)-6-fluoro-8-(4-methyl-piperazin-1-yl)-chroman-2-carboxylic acid hydrochloride (Reference Example 12) and 4-(4-acetylpiperazin-1-yl) aniline. LC/MS (M+1), m/z: 496. ¹H-NMR (300 MHz, CDCl₃), δ 8.63 (s, 1H), 7.49 (d, 2H), 6.92 (d, 2H), 6.57 (dd, 1H), 6.48 (dd, 1H), 4.68 (dd, 1H), 3.77 (t, 2H), 3.62 (t, 2H), 3.31 (bm, 2H), 3.14 (m, 4H), 2.99-2.54 (m, 9H), 2.38 (s, 3H), 2.14 (s, 3H), 2.02 (m, 1H).

### Example 17B

(−)-N-(4-(4-acetylpiperazin-1-yl)phenyl)-6-fluoro-8-(4-methylpiperazin-1-yl)chroman-2-carboxamide (isomer 2) could be obtained in a similar manner as Example 42, but using any of the following methods and columns including: Preparative HPLC using 21×250 mm columns (˜20 mL/min flow rates) with column packings including Chiralpak AD, Chiralpak OD or Chiralpak OJ and mixtures of either EtOH and Hexane or isopropanol and hexane or supercritical fluid chromatography using columns such as Chiracel OD or Chiracel OD-H with either methanol/CO2/dimethylethylamine or ethanol/CO2/dimethylethylamine.

### Example 21

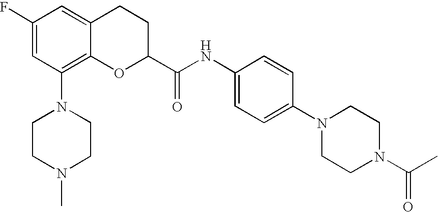

### N-[4-(4-acetylpiperazin-1-yl)phenyl]-6-fluoro-8-(4-methylpiperazin-1-yl)chroman-2-carboxamide

The title compound was prepared in 78% yield in the same manner as Example 17, but using 6-fluoro-8-(4-methyl-piperazin-1-yl) chroman-2-carboxylic acid hydrochloride (Reference Example 15) and 4-(4-acetylpiperazin-1-yl)aniline. LC/MS (M+1), m/z: 496. ^{H-NMR (}300 MHz, CDCl₃), δ 8.63 (s, 1H), 7.49 (d, 2H), 6.92 (d, 2H), 6.57 (d, 1H), 6.48 (d, 1H), 4.68 (dd, 1H), 3.77 (t, 2H), 3.62 (t, 2H), 3.31 (bm, 2H), 3.13 (m, 4H), 2.99-2.55 (m, 9H), 2.38 (s, 3H), 2.14 (s, 3H), 2.03 (m, 1H).

### Example 32

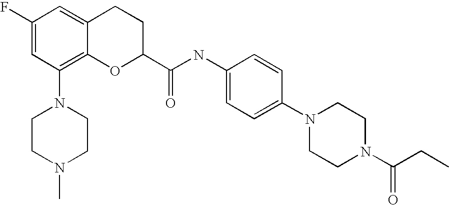

### 6-fluoro-8-(4-methylpiperazin-1-yl)-N-(4-(4-proprionyl-piperazin-1-yl)phenyl)chroman-2-carboxamide

### Example 32A

### 6-fluoro-8-(4-methylpiperazin-1-yl)-N-(4-(4-propionyl-piperazin-1-yl)phenyl)-4-oxo-4H-chromene-2-carboxamide

Prepared as in Example 31A except using 1-[4-(4-amino-phenyl)-piperazin-1-yl]-propan-1-one instead of 4-(4-methyl-piperazin-1-yl)-benzylamine.

### Example 32B

### 6-fluoro-4-hydroxy-8-(4-methylpiperazin-1-yl)-N-(4-(4-propionyl-piperazin-1-yl)phenyl)chroman-2-carboxamide

Prepared as in Example 31B except using 6-fluoro-8-(4-methylpiperazin-1-yl)-N-(4-(4-propionyl-piperazin-1-yl)phenyl)-4-oxo-4H-chromene-2-carboxamide (Example 32A) instead of 6-fluoro-8-(4-methylpiperazin-1-yl)-N-(4-(4-methylpiperazin-1-yl)benzyl)-4-oxo-4H-chromene-2-carboxamide.

### Example 32C

### 6-fluoro-8-(4-methylpiperazin-1-yl)-N-(4-(4-proprionyl-piperazin-1-yl)phenyl)chroman-2-carboxamide

A solution of 6-fluoro-4-hydroxy-8-(4-methylpiperazin-1-yl)-N-(4-(4-propionyl-piperazin-1-yl)phenyl)chroman-2-carboxamide (Example 32B, 0.738 g) in trifluroacetic acid (50 mL) was refluxed for 15 min and concentrated under reduced pressure. Resulting material was dissolved in ethanol (75 mL) and treated with 10% Pd/C and hydrogenated at 40 psi hydrogen pressure for 16 h. At the end of this period the reaction mixture was filtered through diatomaceous earth and concentrated under reduced pressure. This material was dissolved in dichloromethane and washed with aqueous potassium carbonate solution. The material thus obtained was purified by column chromatography over silica gel eluting with 9:1:0.1 mixture of dichloromethane:methanol:dimethylamine yielding the desired product (0.25 g) as a solid.

mp: 160-165 C. LC/MS (M+1), m/z=510. ¹H NMR (300.132 MHz, CDCl3), δ 8.64 (s, 1H), 7.49 (d, J=8.9 Hz, 2H), 6.92 (d, J=9.0 Hz, 2H), 6.57 (q, J=4.4 Hz, 1H), 6.48 (q, J=3.7 Hz, 1H), 4.68 (q, J=4.6 Hz, 1H), 3.75 (1, br s, 2H), 3.60 (1, br s, 2H), 3.31-3.12 (m, 6H), 2.97-2.52 (m, 9H), 2.43-2.35 (m, 4H), 2.08-1.94 (m, 1H), 1.18 (t, J=7.4 Hz, 3H).

### Examples 32D and 32E

The enantiomers could be separated using any of the following methods and columns including: Preparative HPLC using 21×250 mm columns (˜20 mL/min flow rates) with column packings including Chiralpak AD, Chiralpak OD or Chiralpak OJ and mixtures of either EtOH and Hexane or isopropanol and hexane or supercritical fluid chromatography using columns such as Chiracel OD or Chiracel OD-H with either methanol/CO2/dimethylethylamine or ethanol/CO2/dimethylethylamine yielding (+)-6-fluoro-8-(4-methylpiperazin-1-yl)-N-(4-(4-proprionyl-piperazin-1-yl)phenyl)chroman-2-carboxamide and (−)-6-fluoro-8-(4-methylpiperazin-1-yl)-N-(4-(4-proprionyl-piperazin-1-yl)phenyl)chroman-2-carboxamide.

### Example 35

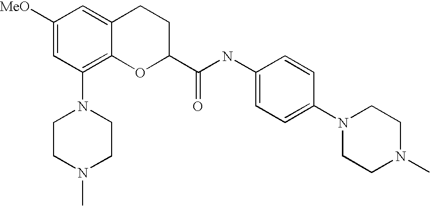

### 6-methoxy-8-(4-methylpiperazin-1-yl)-N-(4-(4-methylpiperazin-1-yl)phenyl)chroman-2-carboxamide

A solution of 6-methoxy-8-(4-methyl-piperazin-1-yl) chroman-2-carboxylic acid hydrochloride (Reference Example 14, 0.306 g) in dichloromethane (10 mL) was treated with N,N-dimethylformamide (2 drops) followed by oxalyl chloride (1.5 mL) and stirred for 16 h. At the end of this period the reaction mixture was concentrated under reduced pressure and the residue was dissolved in dichloromethane (20 mL), cooled 0 C and treated with 4-(N-methylpiperizino) aniline followed by N,N-diisopropylamine (3 mmol). Upon stirring for 16 h at room temperature the reaction mixture was diluted with dichloromethane and washed with sodium carbonate solution. The organic layer was dried over anhydrous potassium carbonate and concentrated under reduced pressure. The resulting product was purified by chromatography over silica gel. Elution with 9:1 dichloromethane: methanol afforded the desired material as a solid (0.232 g). mp 180-190° C., LC/MS 480 (M+1).

¹H NMR (300.132 MHz, DMSO), δ 9.41 (s, 1H), 7.44 (d, J=9.0 Hz, 2H), 6.92 (d, J=14.6 Hz, 2H), 6.30 (s, 2H), 4.61 (dd, J=9.4, 3.1 Hz, 1H), 3.66 (s, 2H), 3.31 (s, 4H), 3.12-2.94 (m, 7H), 2.89-2.66 (m, 3H), 2.51-2.45 (m, 8H), 2.22-2.20 (m, 4H), 2.02-1.90 (m, 1H).

### Examples 35A and 35B

The enantiomers could be separated using any of the following methods and columns including: Preparative HPLC using 21×250 mm columns (˜20 mL/min flow rates) with column packings including Chiralpak AD, Chiralpak OD or Chiralpak OJ and mixtures of either EtOH and Hexane or isopropanol and hexane or supercritical fluid chromatography using columns such as Chiracel OD or Chiracel OD-H with either methanol/CO2/dimethylethylamine or ethanol/CO2/dimethylethylamine yielding (+)-6-methoxy-8-(4-methylpiperazin-1-yl)-N-(4-(4-methylpiperazin-1-yl)phenyl)chroman-2-carboxamide and (−)-6-methoxy-8-(4-methylpiperazin-1-yl)-N-(4-(4-methylpiperazin-1-yl)phenyl)chroman-2-carboxamide.

### Example 36

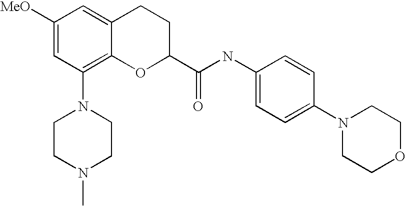

### 6-methoxy-8-(4-methylpiperazin-1-yl)-N-(4-morpholinophenyl)chroman-2-carboxamide

A solution of racemic 6-methoxy-8-(4-methyl-piperazin-1-yl) chroman-2-carboxylic acid (Reference Example 14, 0.306 g) was suspended in dichloromethane (25 mL), cooled to 0 C and treated with N,N-dimethylformamide (2 drops) followed by oxalyl chloride (2 mL). The reaction mixture was allowed to warm to room temperature and stirred for 16 h. At the end of this period, the reaction mixture was concentrated under reduced pressure, treated with toluene (10 mL) and concentrated under reduced pressure. The resulting material was dissolved in dichloromethane (20 mL), cooled to 0 C and treated with 4-morpholino aniline (0.50 g) followed by triethylamine (2.2 mL). The reaction mixture stirred for 15 min at 0 C and then allowed to warm to room temperature. Upon stirring for 1 h the reaction mixture was washed with 10% aqueous potassium carbonate and the aqueous layer was extracted with dichloromethane (60 mL). The combined organic layers dried over anhydrous potassium carbonate, concentrated under reduced pressure and the resulting material was dissolved in methanol (50 mL). Upon refluxing over activated charcoal for 30 min, the reaction mixture was filtered through diatomaceous earth and the diatomaceous earth was washed three times with methanol. The combined filtrates are concentrated under reduced pressure, purified by column chromatography over silica gel eluting with 20:1 dichloromethane:methanol yielding the desired product (0.280 g) as a solid mp>200, LC/MS 467 (M+1). ¹H NMR (300.132 MHz, DMSO), δ 9.43 (s, 1H), 7.46 (d, J=9.0 Hz, 2H), 6.92 (d, J=9.1 Hz, 2H), 6.30 (s, 2H), 4.62 (dd, J=9.4, 3.2 Hz, 1H), 3.73 (t, J=4.7 Hz, 6H), 3.66 (s, 4H), 3.66 (s, 4H), 3.07-2.94 (m, 6H), 2.86-2.67 (m, 1H), 2.51-2.46 (m, 4H), 2.03-1.90 (m, 1H).

### Example 41

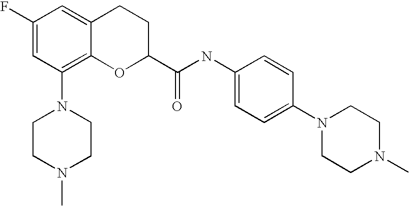

### 6-fluoro-8-(4-methylpiperazin-1-yl)-N-(4-(4-methylpiperazin-1-yl)phenyl)chroman-2-carboxamide

Prepared as Example 36 but using 4-(N-methyl-piperizino) aniline in place of 2-methoxy-5-aminopyridine and using racemic 6-Fluoro-8-(4-methyl-piperazin-1-yl)-chroman-2-carboxylic acid hydrochloride (Reference Example 15) instead of 6-methoxy-8-(4-methyl-piperazin-1-yl) chroman-2-carboxylic acid hydrochloride. Purification of the product by column chromatography over silica gel eluting with 9:1 dichloromethane:methanol yielded the title compound (0.215 g, 61% yield) as a solid. mp 150-160 C, LC/MS 468 (M+1). ¹H NMR (300.132 MHz, DMSO), δ 9.50 (s, 1H), 7.46-7.43 (m, 2H), 6.92-6.84 (m, 2H), 6.58-6.53 (m, 2H), 4.67 (dd, J=9.1, 3.2 Hz, 1H), 3.30 (s, 4H), 3.10-2.92 (m, 7H), 2.86-2.68 (m, 2H), 2.51-2.49 (m, 6H), 2.30-2.13 (m, 6H), 2.05-1.92 (m, 1H).

### Examples 41A and 41B

The enantiomers could be separated using any of the following methods and columns including: Preparative HPLC using 21×250 mm columns ˜20 mL/min flow rates) with column packings including Chiralpak AD, Chiralpak OD or Chiralpak OJ and mixtures of either EtOH and Hexane or isopropanol and hexane or supercritical fluid chromatography using columns such as Chiracel OD or Chiracel OD-H with either methanol/CO2/dimethylethylamine or ethanol/CO2/yielding (+)-6-fluoro-8-(4-methylpiperazin-1-yl)-N-(4-(4-methylpiperazin-1-yl)phenyl)chroman-2-carboxamide and (−)-6-fluoro-8-(4-methylpiperazin-1-yl)-N-(4-(4-methylpiperazin-1-yl)phenyl)chroman-2-carboxamide.

### Example 42

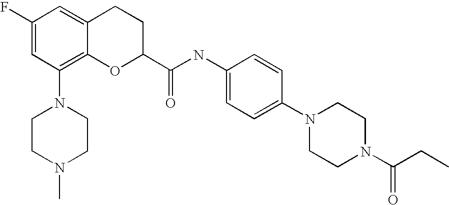

### (+)-6-fluoro-8-(4-methylpiperazin-1-yl)-N-(4-(4-propanoylpiperazin-1-yl)phenyl)chroman-2-carboxamide and (−)6-fluoro-8-(4-methylpiperazin-1-yl)-N-(4-(4-propanoylpiperazin-1-yl)phenyl)chroman-2-carboxamide

A solution of racemic 6-fluoro-8-(4-methyl-piperazin-1-yl) chroman-2-carboxylic acid hydrochloride (Reference Example 15, 2.0 g) was suspended in dichloromethane (100 mL), cooled to 0 C and treated with N,N-dimethylformamide (5 drops) followed by oxalyl chloride (10 mL). The reaction mixture was allowed to warm to the room temperature and stirred for 16 h. At the end of this period, the reaction mixture was concentrated under reduced pressure, treated with toluene (10 mL) and concentrated under reduced pressure. The resulting material was dissolved in dichloromethane (100 mL), cooled to 0 C and treated with 1-[4-(4-amino-phenyl)-piperazin-1-yl]-propan-1-one (1.54 g) followed by triethylamine (4.2 mL). The reaction mixture stirred for 15 min at 0 C and then allowed to warm to the room temperature. Following stirring for 1 h the reaction mixture was washed with 10% aqueous potassium carbonate and the aqueous layer was extracted with dichloromethane (100 mL). The combined organic layers are dried over anhydrous potassium carbonate, concentrated under reduced pressure and the resulting material was dissolved in methanol (50 mL). Upon refluxing over activated charcoal for 30 min, the reaction mixture was filtered through diatomaceous earth and the diatomaceous earth was washed three times with methanol. The combined filtrates are concentrated under reduced pressure and crystalized from methanol to afford a white solid (1.42 g).

### Examples 42A snf 42B

This material was subjected to chiral separation using supercritical fluid chromatography (Chiracel OD-H, 45% MeOH/CO₂+0.5% Dimethylethylamine) to afford 0.60 g of (+) Isomer 1. LC/MS (M+1), m/z 510 ¹H NMR (300.132 MHz, CDCl3), δ 8.66 (s, 1), 7.49 (d, J=8.9 Hz, 2H), 6.92 (d, J=9.0 Hz, 2H), 6.57 (q, J=4.4 Hz, 1H), 6.48 (q, J=3.7 Hz, 1H), 4.68 (q, J=4.6 Hz, 1H), 3.70 (d, J=48.0 Hz, 4H), 3.32-3.10 (m, 6H), 2.97-2.52 (m, 8H), 2.43-2.36 (m, 6H), 2.08-1.94 (m, 1H), 1.18 (t, J=7.4 Hz, 3H). Specific rotation=83° (EtOH, c=1).

The slower moving peak in the above experiment afforded 0.612 g of the (−) isomer. Isomer 2. LC/MS (M+1), m/z 510 ¹H NMR (300.132 MHz, CDCl3), δ 8.66 (s, 1H), 7.49 (d, J=8.9 Hz, 2H), 6.92 (d, J=9.0 Hz, 2H), 6.57 (q, J=4.4 Hz, 1H), 6.48 (q, J=3.7 Hz, 1H), 4.68 (q, J=4.6 Hz, 1H), 3.70 (d, J=48.2 Hz, 4H), 3.32-3.12 (m, 6H), 2.97-2.73 (m, 4H), 2.69-2.52 (m, 4H), 2.43-2.35 (m, 6H), 2.08-1.94 (m, 1H), 1.18 (t, J=7.4 Hz, 3H). Specific rotation=−76° (EtOH, c=1).

### Example 47

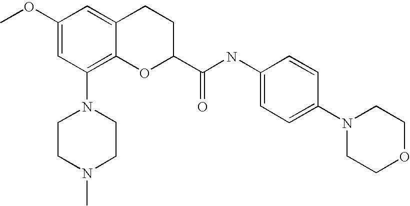

### (+)-6-methoxy-8-(4-methylpiperazin-1-yl)-N-(4-morpholinophenyl)chroman-2-carboxamide

(+)-6-methoxy-8-(4-methyl-piperazin-1-yl) chroman-2-carboxylic acid hydrochloride (Reference Example 19, 10.4 mmol) was dissolved in anhydrous N,N-dimethylformamide (30 ml) and the following are added in order: N,N-diisopropylethylamine (5.44 ml, 31.2 mmol) and TBTU (4.34 g, 13.51 mmol). After stirring for 5 min at room temperature, 4-(4-morpholinyl) aniline (1.84 g, 10.35 mmol) was added and the reaction was stirred overnight at room temperature.

The solution was concentrated in vacuo, the residue was partitioned between ethyl acetate/saturated sodium bicarbonate, extracted with (×2) ethyl acetate. Combined ethyl acetate extracts are washed with: saturated sodium bicarbonate then saturated brine. The washed ethyl acetate extract was dried (MgSO₄) and concentrated in vacuo to give the product.

The product was purified by silica gel chromatography eluting with 99:1 methylene chloride/methanol containing 0.1% ammonium hydroxide then 97:3 methylene chloride/methanol containing 0.1% ammonium hydroxide. The product was collected to give a white solid. This solid was dissolved in methylene chloride (10 ml) Add ether (100 ml) and let sit at room temperature for 3.0 hours. The crystalline solid was collected and dried under oil pump vacuum at 50° C. yielding the title compound as an off-white solid. (4.59 g, 95% yield) LC/MS (M+1), m/z=467. ¹H-NMR (300 MHz, CDCl₃), δ 8.66 (s, 1H), 7.48 (d, 2H, J=9 Hz), 6.90 (d, 2H, J=9 Hz), 6.44 (d, 1H, J=2.7 Hz), 6.29 (d, 1H, J=2.7 Hz), 4.66 (dd, 1H, J₁=9.9 Hz, J₂=3.6 Hz), 3.86 (dd, 4H, J=4.8 Hz), 3.75 (s, 3H), 3.30 (bm, 2H), 3.13 (dd, 4H, J=4.8 Hz), 2.97 (bm, 2H), 2.88 (m, 2H), 2.66 (bm, 4H), 2.59 (m, 1H), 2.38, (s, 3H), 2.01 (m, 1H).

### Example 51

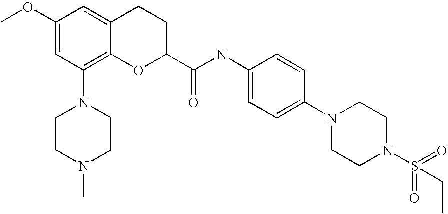

### N-(4-(4-(ethylsulfonyl)piperazin-1-yl)phenyl)-6-methoxy-8-(4-methylpiperazin-yl)chroman-2-carboxamide

6-methoxy-8-(4-methyl-piperazin-1-yl) chroman-2-carboxylic acid hydrochloride (Reference Example 14, 600 mg, 2 mmol) was dissolved in anhydrous N,N-dimethylformamide (6 ml) and the following are added in order: hydroxybenztriazole (351 mg, 2.6 mmol) and TBTU (835 mg, 2.6 mmol). After stirring for 15 min at room temperature, 4-(4-ethanesulfonyl-piperazin-1-yl)-phenylamine (Reference Example 20, 600 mg, 2.2 mmol) was added and the reaction was stirred overnight at room temperature. The solution was concentrated in vacuo, the residue was partitioned between ethyl acetate/1N HCl, extracted with (×2) 1N HCl. Methylene chloride was then added to the combined HCl extracts and small portions of solid sodium bicarbonate added until basic. The aqueous layer was then extracted (×3) with additional methylene chloride. Combined methylene chloride extracts are washed with: saturated sodium bicarbonate then saturated brine. The washed methylene chloride extract was dried (MgSO₄) and concentrated in vacuo to give the crude product. The crude product was purified by solidification in ethyl acetate (10 mL). The resulting solid was collected by filtration and recrystallized from ethyl acetate then isopropanol and finally ethyl acetate. The crystalline solid was collected and dried under oil pump vacuum at 50° C. yielding the title compound as an off-white solid. (300 mg, 27%)

LC/MS (M+1), m/z=557. ¹H-NMR (300 MHz, CDCl₃), δ 8.61 (s, 1H), 7.42 (d, 2H, J=9 Hz), 6.90 (d, 2H, J=9 Hz), 6.57 (dd, J=10.3, 2.9 Hz, 1H), 6.48 (dd, J=8.3, 2.9 Hz, 1H), 4.67 (dd, 1H, J₁=9.9 Hz, J₂=3.6 Hz), 3.76 (s, 3H), 3.46 (dd, 4H, J=5.1 Hz), 3.31 (bm, 2H), 3.22 (dd, 4H, J=5.1 Hz), 2.99 (m, 4H), 2.79 (bm, 2H), 2.65 (bm, 5H), 2.38, (s, 3H), 2.01 (m, 1H), 1.43 (t, 3H, J=7.4 Hz).

### Examples 51A and 51B

The enantiomers could be separated using any of the following methods and columns including: Preparative HPLC using 21×250 mm columns (˜20 mL/min flow rates) with column packings including Chiralpak AD, Chiralpak OD or Chiralpak OJ and mixtures of either EtOH and Hexane or isopropanol and hexane or supercritical fluid chromatography using columns such as Chiracel OD or Chiracel OD-H with either methanol/CO2/dimethylethylamine or ethanol/CO2/dimethylethylamine yielding (+)-N-(4-(4-(ethylsulfonyl)piperazin-1-yl)phenyl)-6-methoxy-8-(4-methylpiperazin-1-yl)chroman-2-carboxamide and (−)-N-(4-(4-(ethylsulfonyl)piperazin-1-yl)phenyl)-6-methoxy-8-(4-methylpiperazin-1-yl)chroman-2-carboxamide.

### Example 72

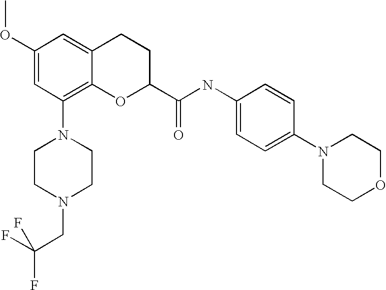

### 6-methoxy-N-(4-morpholinophenyl)-8-(4-(2,2,2-trifluoroethyl)piperazin-1-yl)chroman-2-carboxamide

To a stirred solution of 6-methoxy-8-[4-(2,2,2-trifluoro-ethyl)-piperazin-1-yl]-chroman-2-carboxylic acid (Reference Example 35, 200 mg, 0.52 mmol), 4-morpholin-4-yl-phenylamine (110 mg, 0.62 mmol), and triethylamine (0.30 mL, 2.15 mmol) in 10 mL DCM was added HATU (280 mg, 0.74 mmol). After 18 h the reaction was quenched with 20 mL aqueous 20% K₂CO₃ and extracted with DCM (3×30 mL). Organic extracts are combined, dried over Na₂SO₄, filtered, and evaporated under reduced pressure to give an amber oil that was purified by Prep RPHPLC. [Conditions: 25-90% MeCN (w/0.1% TFA) over 20 minutes, 7 min equilibration at 25% MeCN, 5 min flush at 90% MeCN. Used a 2″ diameter radial compression C8 dynamax 60 Å column]. Residue after evaporation was mixed with 15 mL 20% aqueous K₂CO₃ and extracted with DCM (3×30 mL). Extracts are combined, dried over Na₂SO₄, filtered, and evaporated under reduced pressure to give a solid (31 mg, 12%) product. MS: m/z 535 (M+H). ¹H NMR (300.132 MHz, CDCl₃), δ 8.64 (s, 1H), 7.47 (d, J=8.9 Hz, 2H), 6.90 (d, J=8.9 Hz, 2H), 6.43 (d, J=2.6 Hz, 1H), 6.31 (d, J=2.4 Hz, 1H), 4.94 (dd, J=10.0, 3.7 Hz, 1H), 3.91-3.81 (m, 4H), 3.75 (s, 3H), 3.35-3.23 (m, 2H), 3.00-2.70 (m, 14H), 2.62-2.48 (m, 1H), 2.12-1.93 (m, 1H).

### Examples 72A and 72B

The enantiomers could be separated using any of the following methods and columns including: Preparative HPLC using 21×250 mm columns (˜20 mL/min flow rates) with column packings including Chiralpak AD, Chiralpak OD or Chiralpak OJ and mixtures of either EtOH and Hexane or isopropanol and hexane or supercritical fluid chromatography using columns such as Chiracel OD or Chiracel OD-H with either methanol/CO2/dimethylethylamine or ethanol/CO2/dimethylethylamine yielding (+)-6-methoxy-N-(4-morpholinophenyl)-8-(4-(2,2,2-trifluoroethyl)piperazin-1-yl)chroman-2-carboxamide and (−)-6-methoxy-N-(4-morpholinophenyl)-8-(4-(2,2,2-trifluoroethyl)piperazin-1-yl)chroman-2-carboxamide.

### Example 73

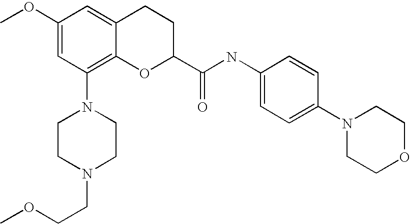

### 6-methoxy-8-(4-(2-methoxyethyl)piperazin-1-yl)-N-(4-morpholinophenyl)chroman-2-carboxamide

4-Morpholin-4-yl-phenylamine (140 mg, 0.77 mmol) was combined with 6-methoxy-8-[4-(2-methoxy-ethyl)-piperazin-1-yl]-chroman-2-carboxylic acid (Reference Example 38, 270 mg, 0.77 mmol) in an analogous fashion to that described in Example 72 to give 41 mg (11%) of product. MS: m/z 469 (M+H). ¹H-NMR (300.132 MHz, CDCl₃), δ 8.68 (s, 1H), 7.48 (d, J=8.9 Hz, 2H), 6.89 (d, J=8.9 Hz, 2H), 6.44 (d, J=2.8 Hz, 1H), 6.29 (d, J=2.7 Hz, 1H), 4.66 (dd, J=10.0, 3.7 Hz, 1H), 3.91-3.81 (m, 4H), 3.74 (s, 3H), 3.55 (t, J=5.5 Hz, 2H), 3.37 (s, 3H), 3.39-3.26 (m, 2H), 3.16-3.08 (m, 4H), 3.05-2.48 (m, 11H), 2.12-1.92 (m, 1H).

### Examples 73A and 73B

The enantiomers could be separated using any of the following methods and columns including: Preparative HPLC using 21×250 mm columns (˜20 mL/min flow rates) with column packings including Chiralpak AD, Chiralpak OD or Chiralpak OJ and mixtures of either EtOH and Hexane or isopropanol and hexane or supercritical fluid chromatography using columns such as Chiracel OD or Chiracel OD-H with either methanol/CO2/dimethylethylamine or ethanol/CO2/dimethylethylamine yielding (+)-6-methoxy-8-(4-(2-methoxyethyl)piperazin-1-yl)-N-(4-morpholinophenyl)chroman-2-carboxamide and (−)-6-methoxy-8-(4-(2-methoxyethyl)piperazin-1-yl)-N-(4-morpholinophenyl)chroman-2-carboxamide.

### Example 75

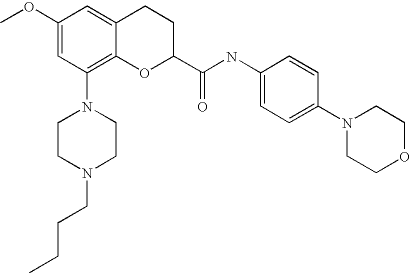

### 8-(4-butylpiperazin-1-yl)-6-methoxy-N-(4-morpholinophenyl)chroman-2-carboxamide

4-Morpholin-4-yl-phenylamine (76 mg, 0.43 mmol) was combined with 8-(4-butyl-piperazin-1-yl)-6-methoxy-chroman-2-carboxylic acid (Reference Example 40, 151 mg, 0.43 mmol) in an analogous fashion to that described in Example 51. Resulting compound needed additional purification by Prep RPHPLC. [Conditions: 25-90% MeCN (w/0.1% TFA) over 20 minutes, 7 min equilibration at 25% MeCN, 5 min flush at 90% MeCN. Used a 2″ diameter radial compression C8 dynamax 60 Å column]. Residue after evaporation was mixed with 15 mL 20% aqueous K₂CO₃ and extracted with DCM (3×30 mL). Extracts are combined, dried over Na₂SO₄, filtered, and evaporated under reduced pressure to give 33 mg (15%) of product. MS: m/z 509 (M+H); ¹H NMR (300.132 MHz, CD₃CN), δ 8.69 (s, 1H), 7.47 (d, J=9.1 Hz, 2H), 6.94 (d, J=9.0 Hz, 2H), 6.43 (d, J=2.8 Hz, 1H), 6.36 (d, J=2.7 Hz, 1H), 4.64 (dd, J=10.0, 4.0 Hz, 1H), 3.85-3.75 (m, 4H), 3.73 (s, 3H), 3.28-3.16 (m, 2H), 3.16-3.04 (m, 4H), 3.01-2.69 (m, 3H), 2.68-2.51 (m, 4H), 2.50-2.34 (m, 1H), 2.38 (t, J=7.3 Hz, 2H), 2.19-2.08 (m, 2H), 1.56-1.27 (m, 4H), 0.95 (t, J=7.2 Hz, 3H).

### Examples 75A and 75B

The enantiomers could be separated using any of the following methods and columns including: Preparative HPLC using 21×250 mm columns (˜20 mL/min flow rates) with column packings including Chiralpak AD, Chiralpak OD or Chiralpak OJ and mixtures of either EtOH and Hexane or isopropanol and hexane or supercritical fluid chromatography using columns such as Chiracel OD or Chiracel OD-H with either methanol/CO2/dimethylethylamine or ethanol/CO2/dimethylethylamine yielding (+)-8-(4-butylpiperazin-1-yl)-6-methoxy-N-(4-morpholinophenyl)chroman-2-carboxamide and (−)-8-(4-butylpiperazin-1-yl)-6-methoxy-N-(4-morpholinophenyl)chroman-2-carboxamide.

### Example 76

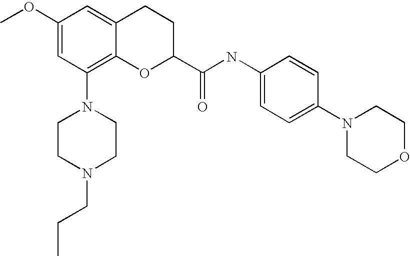

### 8-(4-propylpiperazin-1-yl)-6-methoxy-N-(4-morpholinophenyl)chroman-2-carboxamide

4-Morpholin-4-yl-phenylamine (53 mg, 0.30 mmol) was combined with 6-methoxy-8-(4-propyl-piperazin-1-yl)-chroman-2-carboxylic acid (Reference Example 41, 104 mg, 0.31 mmol) in an analogous fashion to that described in Example 75 to give 31 mg (21%) of product. MS: m/z 495 (M+H); ¹H NMR (300.132 MHz, CDCl₃), δ 8.69 (s, 1H), 7.49 (d, J=8.9 Hz, 2H), 6.90 (d, J=8.9 Hz, 2H), 6.44 (d, J=2.8 Hz, 1H), 6.29 (d, J=2.7 Hz, 1H), 4.66 (dd, J=10.0, 3.8 Hz, 1H), 3.86 (t, J=4.7 Hz, 4H), 3.75 (s, 3H), 3.40-3.23 (m, 2H), 3.19-3.07 (m, 4H), 3.05-2.49 (m, 9H), 2.38 (t, J=7.6 Hz, 2H), 2.15-1.89 (m, 1H), 1.55 (sextet, J=7.4 Hz, 2H), 0.93 (t, J=7.3 Hz, 3H).

### Examples 76A and 76B

The enantiomers could be separated using any of the following methods and columns including: Preparative HPLC using 21×250 mm columns (˜20 mL/min flow rates) with column packings including Chiralpak AD, Chiralpak OD or Chiralpak OJ and mixtures of either EtOH and Hexane or isopropanol and hexane or supercritical fluid chromatography using columns such as Chiracel OD or Chiracel OD-H with either methanol/CO2/dimethylethylamine or ethanol/CO2/dimethylethylamine yielding (+)-8-(4-propylpiperazin-1-yl)-6-methoxy-N-(4-morpholinophenyl)chroman-2-carboxamide and (−)-8-(4-propylpiperazin-1-yl)-6-methoxy-N-(4-morpholinophenyl)chroman-2-carboxamide.

### Example 77

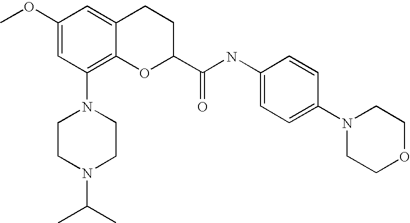

### 8-(4-Isopropylpiperazin-1-yl)-6-methoxy-N-(4-morpholinophenyl)chroman-2-carboxamide

4-Morpholin-4-yl-phenylamine (125 mg, 0.70 mmol) was combined with 8-(4-isopropyl-piperazin-1-yl)-6-methoxy-chroman-2-carboxylic acid (Reference Example 42, 242 mg, 0.72 mmol) in an analogous fashion to that described in Example 51 to give 99 mg (29%) of product. MS: m/z 495 (M+H); ¹H NMR (300.132 MHz, CDCl₃), δ 8.72 (s, 1H), 7.50 (d, J=8.7 Hz, 2H), 6.89 (d, J=8.7 Hz, 2H), 6.44 (d, J=2.5 Hz, 1H), 6.29 (d, J=2.5 Hz, 1H), 4.66 (dd, J=10.2, 3.7 Hz, 1H), 3.94-3.79 (m, 4H), 3.75 (s, 3H), 3.39-3.24 (m, 2H), 3.20-3.06 (m, 4H), 3.05-2.66 (m, 9H), 2.65-2.49 (m, 1H), 2.11-1.90 (m, 1H), 1.10 (d, J=6.4 Hz, 6H).

### Examples 77A and 77B

The enantiomers could be separated using any of the following methods and columns including: Preparative HPLC using 21×250 mm columns (˜20 mL/min flow rates) with column packings including Chiralpak AD, Chiralpak OD or Chiralpak OJ and mixtures of either EtOH and Hexane or isopropanol and hexane or supercritical fluid chromatography using columns such as Chiracel OD or Chiracel OD-H with either methanol/CO2/dimethylethylamine or ethanol/CO2/dimethylethylamine yielding (+)-8-(4-Isopropylpiperazin-1-yl)-6-methoxy-N-(4-morpholinophenyl)chroman-2-carboxamide and (−)-8-(4-Isopropylpiperazin-1-yl)-6-methoxy-N-(4-morpholinophenyl)chroman-2-carboxamide.

### Example 78

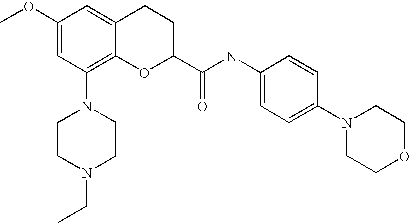

### 8-(4-Ethylpiperazin-1-yl)-6-methoxy-N-(4-morpholinophenyl)chroman-2-carboxamide

4-Morpholin-4-yl-phenylamine (130 mg, 0.73 mmol) was combined with 8-(4-ethyl-piperazin-1-yl)-6-methoxy-chroman-2-carboxylic acid (Reference Example 43, 231 mg, 0.72 mmol) in an analogous fashion to that described in Example 51 to give 70 mg (20%) of product. MS: m/z 481 (M+H); ¹H NMR (300.132 MHz, CDCl₃), δ 8.69 (s, 1H), 7.49 (d, J=8.9 Hz, 2H), 6.89 (d, J=8.9 Hz, 2H), 6.45 (d, J=2.6 Hz, 1H), 6.29 (d, J=2.6 Hz, 1H), 4.66 (dd, J=9.9, 3.6 Hz, 1H), 3.92-3.81 (m, 4H), 3.75 (s, 3H), 3.41-3.24 (m, 2H), 3.19-3.07 (m, 4H), 3.06-2.43 (m, 11H), 2.13-1.91 (m, 1H), 1.13 (t, J=7.1 Hz, 3H).

### Examples 78A and 78B

The enantiomers could be separated using any of the following methods and columns including: Preparative HPLC using 21×250 mm columns (˜20 mL/min flow rates) with column packings including Chiralpak AD, Chiralpak OD or Chiralpak OJ and mixtures of either EtOH and Hexane or isopropanol and hexane or supercritical fluid chromatography using columns such as Chiracel OD or Chiracel OD-H with either methanol/CO2/dimethylethylamine or ethanol/CO2/dimethylethylamine yielding (+)-8-(4-Ethylpiperazin-1-yl)-6-methoxy-N-(4-morpholinophenyl)chroman-2-carboxamide and (−)-8-(4-Ethylpiperazin-1-yl)-6-methoxy-N-(4-morpholinophenyl)chroman-2-carboxamide.

### Example 79

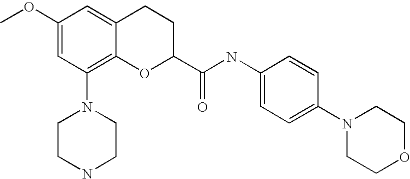

### 6-methoxy-N-(4-morpholinophenyl)-8-(piperazin-1-yl)chroman-2-carboxamide

### Example 79A

### 4-[6-methoxy-2-(4-morpholin-4-yl-phenylcarbamoyl)-chroman-8-yl]-piperazine-1-carboxylic acid tert-butyl ester

4-Morpholin-4-yl-phenylamine (85 mg, 0.48 mmol) was combined with 4-(2-carboxy-6-methoxy-chroman-8-yl)-piperazine-1-carboxylic acid tert-butyl ester (Reference Example 44, 190 mg, 0.48 mmol) in an analogous fashion (except PyAOP was substituted for TBTU) to that described in Example 51 to give 180 mg (67%) of product; ¹H NMR (300.132 MHz, CDCl₃), δ 10.21 (s, 1H), 8.01 (d, J=8.7 Hz, 2H), 7.50 (d, J=8.7 Hz, 2H), 6.81 (d, J=2.7 Hz, 1H), 6.69 (d, J=2.7 Hz, 1H), 5.12-5.05 (m, 11H), 4.63 (d, J=9.2 Hz, 1H), 4.04-3.87 (m, 4H), 3.87-3.29 (m, 8H), 3.75 (s, 3H), 3.01-2.75 (m, 6H), 2.28-1.82 (m, 1H), 1.49 (s, 9H).

### Example 79B

### 8-(Piperazin-1-yl)-6-methoxy-N-(4-morpholinophenyl)chroman-2-carboxamide

To a solution of 4-[6-methoxy-2-(4-morpholin-4-yl-phenylcarbamoyl)-chroman-8-yl]-piperazine-1-carboxylic acid tert-butyl ester (Example 79A, 80 mg, 0.14 mmol) in dichloromethane (5 mL) was added trifluoroacetic acid (1 mL). Reaction was stirred for 1 h. Solvents are evaporated under reduced pressure and product was purified by Prep RPHPLC. [Conditions: 25-90% MeCN (w/0.1% TFA) over 20 minutes; 7 min equilibration at 25% MeCN, 5 min flush at 90% MeCN. Used a 2″ diameter radial compression C8 dynamax 60 Å column]. Residue after evaporation was mixed with 15 mL 20% K₂CO₃ (aq) and extracted with DCM (3×30 mL). Extracts are combined, dried over Na₂SO₄, filtered, and evaporated under reduced pressure to give 17 mg (26%) of product. MS: m/z 453 (M+H); ¹H NMR (300.132 MHz, CD₃CN), δ 8.61 (s, 1H), 7.46 (d, J=9.0 Hz, 1H), 6.94 (d, J=9.0 Hz, 1H), 6.43 (d, J=2.9 Hz, 1H), 6.39 (d, J=2.9 Hz, 1H), 4.64 (dd, J=9.5, 3.6 Hz, 1H), 3.84-3.76 (m, 4H), 3.73 (s, 3H), 3.35-2.91 (m, 14H), 2.88 (dd, J=10.2, 5.2 Hz, 1H), 2.77 (dt, J=16.4, 4.9 Hz, 1H), 2.50-2.36 (m, 1H), 2.11-1.96 (m, 1H).

### Examples 79C and 79D

The enantiomers could be separated using any of the following methods and columns including: Preparative HPLC using 21×250 mm columns (˜20 mL/min flow rates) with column packings including Chiralpak AD, Chiralpak OD or Chiralpak OJ and mixtures of either EtOH and Hexane or isopropanol and hexane or supercritical fluid chromatography using columns such as Chiracel OD or Chiracel OD-H with either methanol/CO2/dimethylethylamine or ethanol/CO2/dimethylethylamine yielding (+)-8-(Piperazin-1-yl)-6-methoxy-N-(4-morpholinophenyl)chroman-2-carboxamide and (−)-8-(Piperazin-1-yl)-6-methoxy-N-(4-morpholinophenyl)chroman-2-carboxamide.

### Example 82

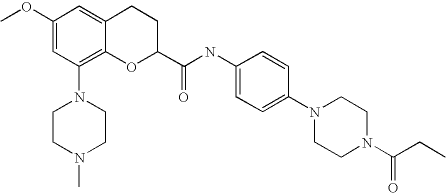

### 6-methoxy-8-(4-methylpiperazin-1-yl)-N-(4-(4-propanoylpiperazin-1-yl)phenyl)chroman-2-carboxamide

This compound was prepared from 6-methoxy-8-(4-methyl-piperazin-1-yl)-chroman-2-carboxylic acid hydrochloride (Reference Example 14) and 1-[4-4-amino-phenyl)-piperazin-1-yl-prpoan-1-one as described in Example 51.

### Examples 82A and 82B

The enantiomers are separated using chiral SFC (Chiralpak AD, 40% IPA/CO₂+0.5% Dimethylethylamine) yielding 6-methoxy-8-(4-methylpiperazin-1-yl)-N-(4-(4-propanoylpiperazin-1-yl)phenyl)chroman-2-carboxamide (Isomer 1, rt=4.40 min) and 6-methoxy-8-(4-methylpiperazin-1-yl)-N-(4-(4-propanoylpiperazin-1-yl)phenyl)chroman-2-carboxamide (Isomer 2, rt=5.22 min).

Isomer 1: LC/MS: 522 (M+H, 100%); ¹H-NMR (300 MHz, DMSO-d₆), δ 9.44 (bs, 1H), 7.26 (d, J=9.25 Hz, 2H), 6.75 (s, 1H), 6.73 (d, J=9.25 Hz, 2H), 6.49 (s, 1H), 4.59 (m, 1H), 3.85 (s, 3H), 3.83 (m, 4H), 3.21 (m, 4H), 2.96 (m, 2H), 2.94 (m, 4H), 2.72 (m, 2H), 2.53 (m, 2H), 2.34 (s, 3H) 2.32 (q, J=7.4 Hz, 2H), 0.97 (t, J=7.4 Hz, 3H).

Isomer 2: LC/MS: 522 (M+H, 100%); ¹H-NMR (300 MHz, DMSO-d₆), δ 9.44 (bs, 1H), 7.26 (d, J=9.25 Hz, 2H), 6.75 (s, 1H), 6.73 (d, J=9.25 Hz, 2H), 6.49 (s, 1H), 4.59 (m, 1H), 3.85 (s, 3H), 3.83 (m, 4H), 3.21 (m, 4H), 2.96 (m, 2H), 2.94 (m, 4H), 2.72 (m, 2H), 2.53 (m, 2H), 2.34 (s, 3H), 2.32 (q, J=7.4 Hz, 2H), 0.97 (t, J=7.4 Hz, 3H).

### Example 83

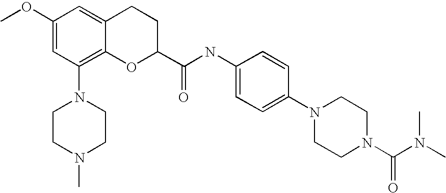

### 4-(4-(6-methoxy-8-(4-methylpiperazin-1-yl)chroman-2-carboxamido)phenyl)-N,N-dimethylpiperazine-1-carboxamide

This compound was prepared from 6-methoxy-8-(4-methyl-piperazin-1-yl)-chroman-2-carboxylic acid hydrochloride (Reference Example 14) and 4-(4-Amino-phenyl)-piperazine-1-carboxylic acid dimethylamide (Reference Example 47) as described in Example 51.

### Examples 83A and 83B

The enantiomers are separated using chiral SFC (Chiralpak AD, 40% IPA/CO₂+0.5% dimethylethylamine) yielding 4-(4-(6-methoxy-8-(4-methylpiperazin-1-yl)chroman-2-carboxamido)phenyl)-N,N-dimethylpiperazine-1-carboxamide (Isomer 1, rt=4.08 min) and 4-(4-(6-methoxy-8-(4-methylpiperazin-1-yl)chroman-2-carboxamido)phenyl)-N,N-dimethylpiperazine-1-carboxamide (Isomer 2, rt=4.98 min).

Isomer 1: LC/MS: 537 (M+H, 100%); ¹H-NMR (300 MHz, DMSO-d₆), δ 9.44 (bs, 1H), 7.26 (d, J=9.25 Hz, 2H), 6.75 (s, 1H), 6.73 (d, J=9.25 Hz, 2H), 6.49 (s, 1H), 4.59 (m, 1H), 3.85 (s, 3H), 3.75 (m, 4H), 3.21 (m, 4H), 2.96 (m, 2H), 2.94 (m, 4H), 2.83 (s, 3H), 2.73 (s, 3H), 2.72 (m, 2H), 2.53 (m, 4H), 2.34 (s, 3H).

Isomer 2: LC/MS: 537 (M+H, 100%); ¹H-NMR (300 MHz, DMSO-d₆), δ 9.44 (bs, 1H), 7.26 (d, J=9.25 Hz, 2H), 6.75 (s, 1H), 6.73 (d, J=9.25 Hz, 2H), 6.49 (s, 1H), 4.59 (m, 1H), 3.85 (s, 3H), 3.75 (m, 4H), 3.21 (m, 4H), 2.96 (m, 2H), 2.94 (m, 4H), 2.83 (s, 3H), 2.73 (s, 3H), 2.72 (m, 2H), 2.53 (m, 4H), 2.34 (s, 3H).

### Example 89

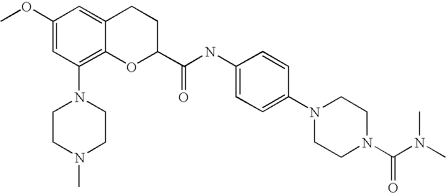

### 4-(4-(6-methoxy-8-(4-methylpiperazin-1-yl)chroman-2-carboxamido)phenyl)-N,N-dimethylpiperazine-1-carboxamide

This compound was prepared in 58% yield from 6-methoxy-8-(4-methyl-piperazin-1-yl)-chroman-2-carboxylic acid hydrochloride (Reference Example 14) and 4-(4-Amino-phenyl)-piperazine-1-carboxylic acid dimethylamide (Reference Example 47) as described in Example 51. LC/MS: 537 (M+H, 100%); ¹H-NMR (300 MHz, DMSO-d₆), δ 9.44 (bs, 1H), 7.26 (d, J=9.3 Hz, 2H), 6.75 (d, J=9.3 Hz, 2H), 6.49 (s, 1H), 4.59 (m, 1H), 3.85 (s, 3H), 3.75 (m, 4H), 3.27 (m, 4H), 3.13 (m, 4H), 2.96 (m, 2H), 2.83 (s, 3H), 2.73 (s, 3H), 2.53 (m, 4H), 2.34 (s, 3H), 2.28 (m, 2H).

### Examples 89A and 89B

The enantiomers could be separated using any of the following methods and columns including: Preparative HPLC using 21×250 mm columns (˜20 mL/min flow rates) with column packings including Chiralpak AD, Chiralpak OD or Chiralpak OJ and mixtures of either EtOH and Hexane or isopropanol and hexane or supercritical fluid chromatography using columns such as Chiracel OD or Chiracel OD-H with either methanol/CO2/dimethylethylamine or ethanol/CO2/dimethylethylamine yielding (+)-4-(4-(6-methoxy-8-(4-methylpiperazin-1-yl)chroman-2-carboxamido)phenyl)-N,N-dimethylpiperazine-1-carboxamide and (−)-4-(4-(6-methoxy-8-(4-methylpiperazin-1-yl)chroman-2-carboxamido)phenyl)-N,N-dimethylpiperazine-1-carboxamide.

### Example 90

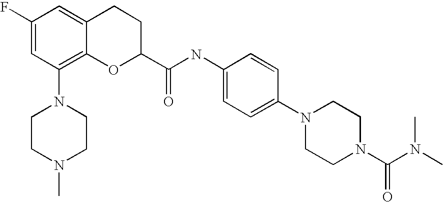

### 4-(4-(6-fluoro-8-(4-methylpiperazin-1-yl)chroman-2-carboxamido)phenyl)-N,N-dimethylpiperazine-1-carboxamide

This compound was prepared in 64% yield from 6-fluoro-8-(4-methyl-piperazin-1-yl)-chroman-2-carboxylic acid hydrochloride (Reference Example 15) and 4-(4-Amino-phenyl)-piperazine-1-carboxylic acid dimethylamide (Reference Example 47) as described in Example 51. LC/MS: 525 (M+H, 100%); ¹H-NMR (300 MHz, DMSO-d₆), δ 9.44 (bs, 1H), 7.26 (d, J=9.3 Hz, 2H), 6.75 (d, J=9.3 Hz, 2H), 6.65 (d, J=8.9 Hz, 1H), 6.41 (d, J=11.7 Hz, 1H), 4.60 (m, 1H), 3.75 (m, 4H), 3.27 (m, 4H), 3.22 (m, 4H), 3.03 (m, 2H), 2.83 (s, 3H), 2.73 (s, 3H), 2.54 (m, 4H), 2.34 (s, 3H), 2.29 (m, 2H).

### Examples 90A and 90B

The enantiomers could be separated using any of the following methods and columns including: Preparative HPLC using 21×250 mm columns (˜20 mL/min flow rates) with column packings including Chiralpak AD, Chiralpak OD or Chiralpak OJ and mixtures of either EtOH and Hexane or isopropanol and hexane or supercritical fluid chromatography using columns such as Chiracel OD or Chiracel OD-H with either methanol/CO2/dimethylethylamine or ethanol/CO2/dimethylethylamine yielding (+)-4-(4-(6-fluoro-8-(4-methylpiperazin-1-yl)chroman-2-carboxamido)phenyl)-N,N-dimethylpiperazine 1-carboxamide and (−)-4-(4-(6-fluoro-8-(4-methylpiperazin-1-yl)chroman-2-carboxamido)phenyl)-N,N-dimethylpiperazine-1-carboxamide.

### Example 93

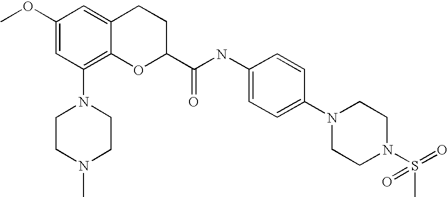

### 6-methoxy-8-(4-methylpiperazin-1-yl)-N-(4-(4-(methylsulfonyl)piperazin-1-yl)phenyl)chroman-2-carboxamide

This compound was prepared in 61% yield from 6-methoxy-8-(4-methyl-piperazin-1-yl)-chroman-2-carboxylic acid hydrochloride (Reference Example 14) and 4-(4-methanesulfonyl-piperazin-1-yl)-phenylamine as described in Example 51. LC/MS: 544 (M+H, 100%); ¹H-NMR (300 MHz, DMSO-d₆), δ 9.44 (bs, 1H), 7.26 (d, J=9.3 Hz, 2H), 6.79 (d, J=9.3 Hz, 2H), 6.65 (d, J=8.9 Hz, 1H), 6.41 (d, J=11.7 Hz, 1H), 4.60 (m, 1H), 3.85 (s, 3H), 3.59 (m, 4H), 3.51 (m, 4H), 3.22 (m, 4H), 3.03 (m, 2H), 2.93 (s, 3H), 2.73 (m, 2H), 2.54 (m, 4H), 2.34 (s, 3H).

### Examples 93A and 93B

The enantiomers could be separated using any of the following methods and columns including: Preparative HPLC using 21×250 mm columns (˜20 mL/min flow rates) with column packings including Chiralpak AD, Chiralpak OD or Chiralpak OJ and mixtures of either EtOH and Hexane or isopropanol and hexane or supercritical fluid chromatography using columns such as Chiracel OD or Chiracel OD-H with either methanol/CO2/dimethylethylamine or ethanol/CO2/yielding (+)-6-methoxy-8-(4-methylpiperazin-1-yl)-N-(4-(4-(methylsulfonyl)piperazin-1-yl)phenyl)chroman-2-carboxamide and (−)-6-methoxy-8-(4-methylpiperazin-1-yl)-N-(4-(4-(methylsulfonyl)piperazin-1-yl)phenyl)chroman-2-carboxamide.

### Example 95

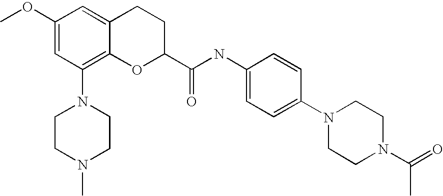

### N-(4-(4-ethanoylpiperazin-1-yl)phenyl)-6-methoxy-8-(4-methylpiperazin-1-yl)chroman-2-carboxamide

This compound was prepared from 6-methoxy-8-(4-methyl-piperazin-1-yl)-chroman-2-carboxylic acid hydrochloride (Reference Example 14) and 1-[4-(4-amino-phenyl)-piperazin-1-yl]-ethanone as described in Example 51.

### Examples 95A and 95B

The enantiomers are separated using chiral SFC (Chiralpak AD, 37% IPA/CO₂+0.5% Dimethylethylamine) yielding N-(4-(4-ethanoylpiperazin-1-yl)phenyl)-6-methoxy-8-(4-methylpiperazin-1-yl)chroman-2-carboxamide (Isomer 1, rt=5.07 min) and N-(4-(4-ethanoylpiperazin-1-yl)phenyl)-6-methoxy-8-(4-methylpiperazin-1-yl)chroman-2-carboxamide (Isomer 2, rt=6.24 min).

Isomer 1: LC/MS: 508 (M+H, 100%); ¹H-NMR (300 MHz, DMSO-d₆), δ 9.44 (bs, 1H), 7.26 (d, J=9.25 Hz, 2H), 6.75 (s, 1H), 6.73 (d, J=9.25 Hz, 2H), 6.49 (s, 1H), 4.59 (m, 1H), 3.85 (s, 3H), 3.62 (m, 2H), 3.45 (m, 2H), 3.21 (m, 4H), 3.09 (m, 2H), 2.96 (m, 4H), 2.89 (m, 2H), 2.72 (m, 2H), 2.53 (m, 2H), 2.44 (s, 3H), 2.34 (s, 3H).

Isomer 2: LC/MS: 508 (M+H, 100%); ¹H-NMR (300 MHz, DMSO-d₆), δ 9.44 (bs, 1H), 7.26 (d, J=9.25 Hz, 2H), 6.75 (s, 1H), 6.73 (d, J=9.25 Hz, 2H), 6.49 (s, 1H), 4.59 (m, 1H), 3.85 (s, 3H), 3.62 (m, 2H), 3.45 (m, 2H), 3.21 (m, 4H), 3.09 (m, 2H), 2.96 (m, 4H), 2.89 (m, 2H), 2.72 (m, 2H), 2.53 (m, 2H), 2.44 (s, 3H), 2.34 (s, 3H).

### Example 96

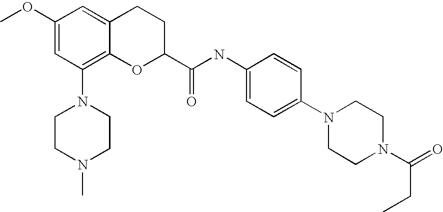

### 6-methoxy-8-(4-methylpiperazin-1-yl)-N-(4-(4-propanoylpiperazin-1-yl)phenyl)chroman-2-carboxamide

This compound was prepared in 53% yield from 6-methoxy-8-(4-methyl-piperazin-1-yl)-chroman-2-carboxylic acid hydrochloride (Reference Example 14) and 1-[4-(4-amino-phenyl)-piperazin-1-yl]-propan-1-one as described in Example 51. LC/MS: 522 (M+H, 100%); ¹H-NMR (300 MHz, DMSO-d₆), δ 9.44 (bs, 1H), 7.26 (d, J=9.25 Hz, 2H), 6.75 (s, 1H), 6.73 (d, J=9.25 Hz, 2H), 6.49 (s, 1H), 4.59 (m, 1H), 3.85 (s, 3H), 3.62 (m, 2H), 3.45 (m, 2H), 3.21 (m, 4H), 3.09 (m, 2H), 2.96 (m, 4H), 2.89 (m, 2H), 2.72 (m, 2H), 2.53 (m, 2H), 2.44 (m, 2H), 2.34 (s, 3H), 0.97 (t, J=7.4 Hz, 3H).

### Examples 96A and 96B

The enantiomers could be separated using any of the following methods and columns including: Preparative HPLC using 21×250 mm columns (˜20 mL/min flow rates) with column packings including Chiralpak AD, Chiralpak OD or Chiralpak OJ and mixtures of either EtOH and Hexane or isopropanol and hexane or supercritical fluid chromatography using columns such as Chiracel OD or Chiracel OD-H with either methanol/CO2/dimethylethylamine or ethanol/CO2/dimethylethylamine yielding (+)-6-methoxy-8-(4-methylpiperazin-1-yl)-N-(4-(4-propanoylpiperazin-1-yl)phenyl)chroman-2-carboxamide and (−)-6-methoxy-8-(4-methylpiperazin-1-yl)-N-(4-(4-propanoylpiperazin-1-yl)phenyl)chroman-2-carboxamide.

### Example 103

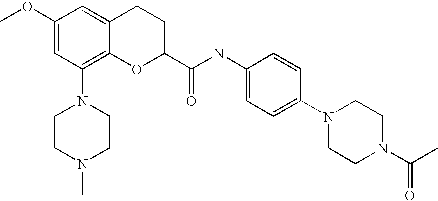

### N-(4-(4-ethanoylpiperazin-1-yl)phenyl)-6-methoxy-8-(4-methylpiperazin-1-yl)chroman-2-carboxamide

This compound was prepared in 60% yield from 6-methoxy-8-(4-methyl-piperazin-1-yl)-chroman-2-carboxylic acid hydrochloride (Reference Example 14) and 1-[4-(4-amino-phenyl)-pierazin-1-yl]-ethanone as described in Example 5. LC/MS: 508 (M+H, 100%); ¹H-NMR (300 MHz, DMSO-d₆), δ 9.44 (bs, 1H), 7.26 (d, J=9.25 Hz, 2H), 6.75 (s, 1H), 6.73 (d, J=9.25 Hz, 2H), 6.49 (s, 1H), 4.59 (m, 1H), 3.85 (s, 3H), 3.62 (m, 2H), 3.45 (m, 2H), 3.21 (m, 4H), 3.09 (m, 2H), 2.96 (m, 4H), 2.89 (m, 2H), 2.72 (m, 2H), 2.53 (m, 2H), 2.44 (s, 3H), 2.34 (s, 3H).

### Examples 103A and 103B

The enantiomers could be separated using any of the following methods and columns including: Preparative HPLC using 21×250 mm columns (˜20 mL/min flow rates) with column packings including Chiralpak AD, Chiralpak OD or Chiralpak OJ and mixtures of either EtOH and Hexane or isopropanol and hexane or supercritical fluid chromatography using columns such as Chiracel OD or Chiracel OD-H with either methanol/CO2/dimethylethylamine or ethanol/CO2/dimethylethylamine yielding (+)-N-(4-(4-ethanoylpiperazin-1-yl)phenyl)-6-methoxy-8-(4-methylpiperazin-1-yl)chroman-2-carboxamide and (−)-N-(4-(4-ethanoylpiperazin-1-yl)phenyl)-6-methoxy-8-(4-methylpiperazin-1-yl)chroman-2-carboxamide.

Compounds of the present invention include, but are not limited to, the following compositions listed in Table 1 on the following pages.

TABLE 1
Compounds.

| Ex. | | |
|---|---|---|
| No. | Structure | Compound Name |
| 2 | 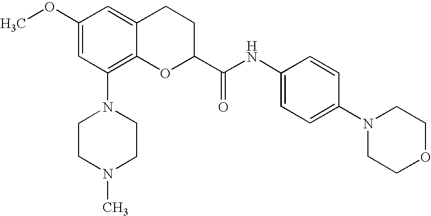 | (−)-6-methoxy-8-(4- methylpiperazin-1-yl)-N-(4- morpholinophenyl)chroman- 2-carboxamide (Isomer 2) |
| 17 | 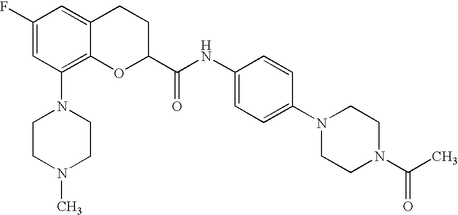 | (+)-N-(4-(4-acetylpiperazin- 1-yl)phenyl)-6-fluoro-8-(4- methylpiperazin-1- yl)chroman-2-carboxamide (Isomer 1) |
| 17B | 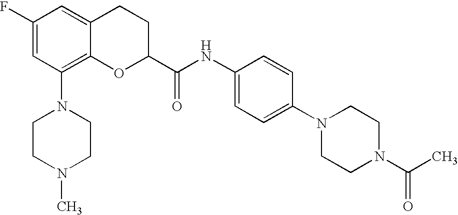 | (−)-N-(4-(4-acetylpiperazin- 1-yl)phenyl)-6-fluoro-8-(4- methylpiperazin-1- yl)chroman-2-carboxamide (Isomer 2) |
| 21 | 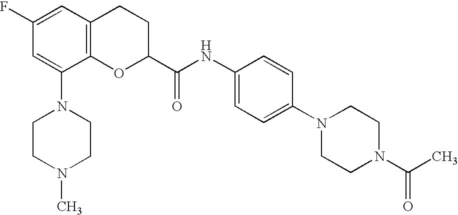 | N-(4-(4-ethanoylpiperazin-1- yl)phenyl)-6-fluoro-8-(4- methylpiperazin-1- yl)chroman-2-carboxamide |
| 32 | 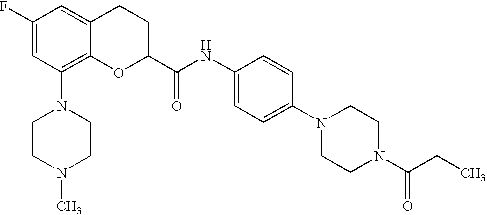 | 6-fluoro-8-(4- methylpiperazin-1-yl)-N-(4- (4-propanoylpiperazin-1- yl)phenyl)chroman-2- carboxamide |
| 32D | 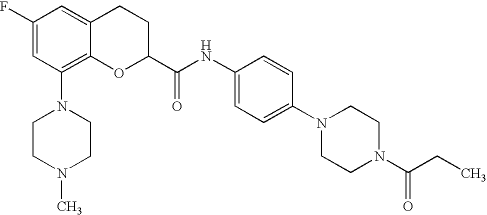 | (+)-6-fluoro-8-(4- methylpiperazin-1-yl)-N-(4- (4-propanoylpiperazin-1- yl)phenyl)chroman-2- carboxamide (Isomer 1) |
| 32E | 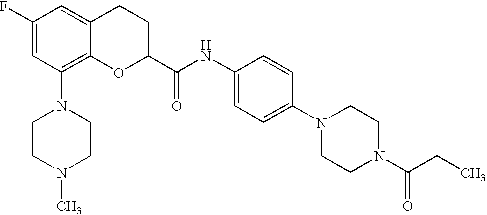 | (−)-6-fluoro-8-(4- methylpiperazin-1-yl)-N-(4- (4-propanoylpiperazin-1- yl)phenyl)chroman-2- carboxamide (Isomer 2) |
| 35 | 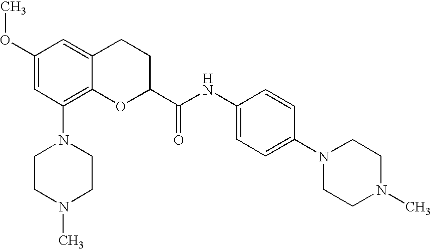 | 6-methoxy-8-(4- methylpiperazin-1-yl)-N-(4- (4-methylpiperazin-1- yl)phenyl)chroman-2- carboxamide |
| 35A | 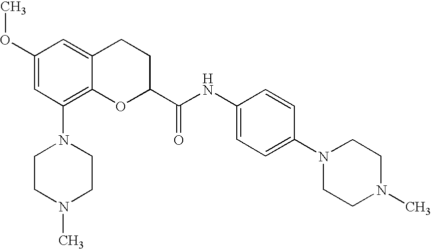 | (+)-6-methoxy-8-(4- methylpiperazin-1-yl)-N-(4- (4-methylpiperazin-1- yl)phenyl)chroman-2- carboxamide (Isomer 1) |
| 35B | 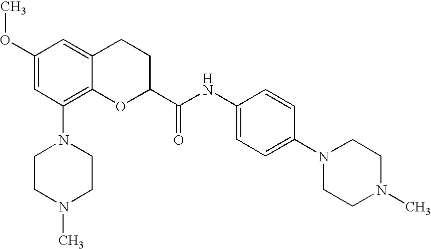 | (−)-6-methoxy-8-(4- methylpiperazin-1-yl)-N-(4- (4-methylpiperazin-1- yl)phenyl)chroman-2- carboxamide (Isomer 2) |
| 36 | 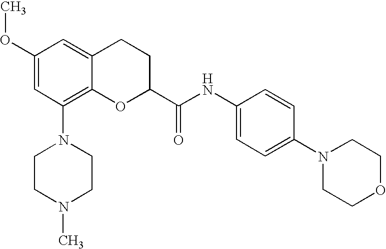 | 6-methoxy-8-(4- methylpiperazin-1-yl)-N-(4- morpholinophenyl)chroman- 2-carboxamide |
| 41 | 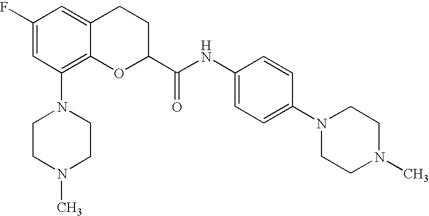 | 6-fluoro-8-(4- methylpiperazin-1-yl)-N-(4- (4-methylpiperazin-1- yl)phenyl)chroman-2- carboxamide |
| 41A | 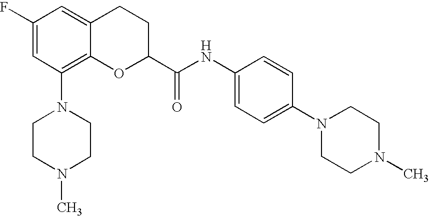 | (+)-6-fluoro-8-(4- methylpiperazin-1-yl)-N-(4- (4-methylpiperazin-1- yl)phenyl)chroman-2- carboxamide (Isomer 1) |
| 41B | 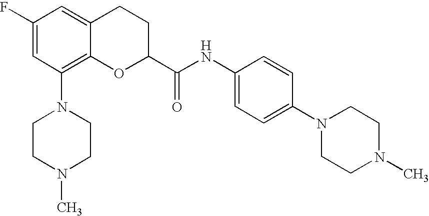 | (−)-6-fluoro-8-(4- methylpiperazin-1-yl)-N-(4- (4-methylpiperazin-1- yl)phenyl)chroman-2- carboxamide (Isomer 2) |
| 42 | 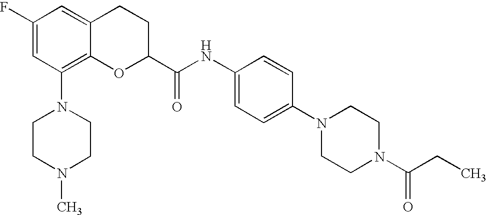 | 6-fluoro-8-(4- methylpiperazin-1-yl)-N-(4- (4-propanoylpiperazin-1- yl)phenyl)chroman-2- carboxamide |
| 42A | 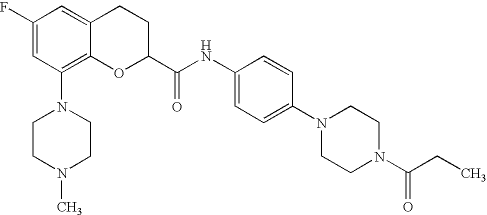 | (+)-6-fluoro-8-(4- methylpiperazin-1-yl)-N-(4- (4-propanoylpiperazin-1- yl)phenyl)chroman-2- carboxamide (Isomer 1) |
| 42B | 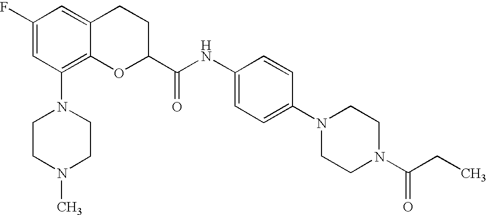 | (−)-6-fluoro-8-(4- methylpiperazin-1-yl)-N-(4- (4-propanoylpiperazin-1- yl)phenyl)chroman-2- carboxamide (Isomer 2) |
| 47 | 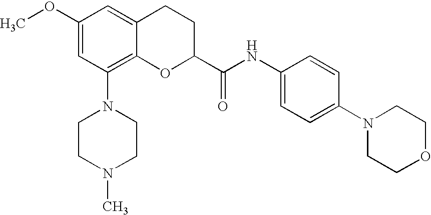 | (R)-(+)-6-methoxy-8-(4- methylpiperazin-1-yl)-N-(4- morpholinophenyl)chroman- 2-carboxamide ((+) Isomer 1) |
| 51 | 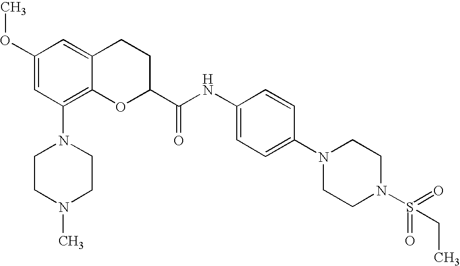 | N-(4-(4- (ethylsulfonyl)piperazin-1- yl)phenyl)-6-methoxy-8-(4- methylpiperazin-1- yl)chroman-2-carboxamide |
| 51A | 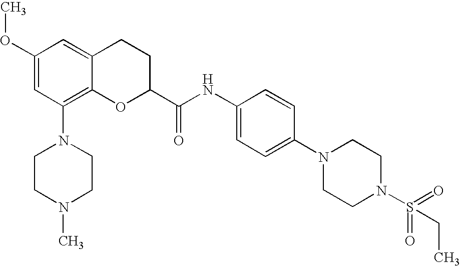 | (+)-N-(4-(4- (ethylsulfonyl)piperazin-1- yl)phenyl)-6-methoxy-8-(4- methylpiperazin-1- yl)chroman-2-carboxamide (Isomer 1) |
| 51B | 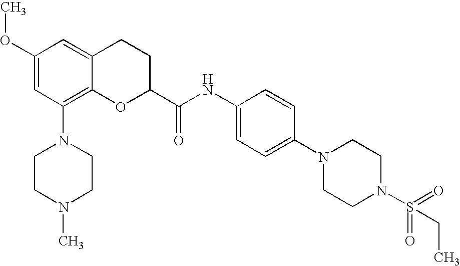 | (−)-N-(4-(4- (ethylsulfonyl)piperazin-1- yl)phenyl)-6-methoxy-8-(4- methylpiperazin-1- yl)chroman-2-carboxamide (Isomer 2) |
| 72 | 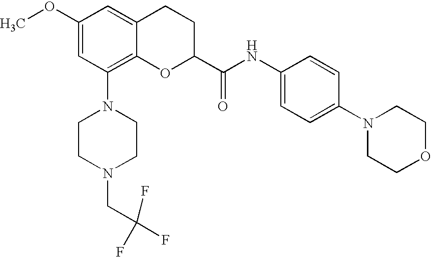 | 6-methoxy-N-(4- morpholinophenyl)-8-(4- (2,2,2- trifluoroethyl)piperazin-1- yl)chroman-2-carboxamide |
| 72A | 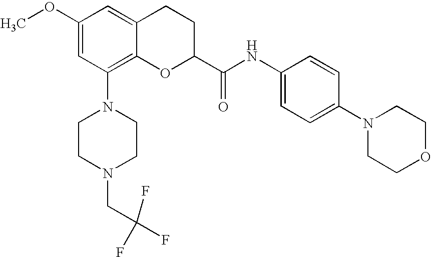 | (+)-6-methoxy-N-(4- morpholinophenyl)-8-(4- (2,2,2- trifluoroethyl)piperazin-1- yl)chroman-2-oarboxamide (Isomer 1) |
| 72B | 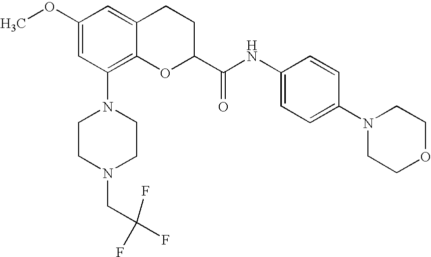 | (−)-6-methoxy-N-(4- morpholinophenyl)-8-(4- (2,2,2- trifluoroethyl)piperazin-1- yl)chroman-2-carboxamide (Isomer 2) |
| 73 | 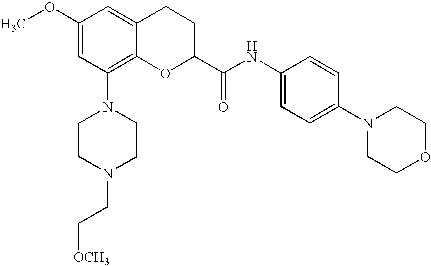 | 6-methoxy-8-(4-(2- methoxyethyl)piperazin-1- yl)-N-(4- morpholinophenyl)chroman- 2-carboxamide |
| 73A | 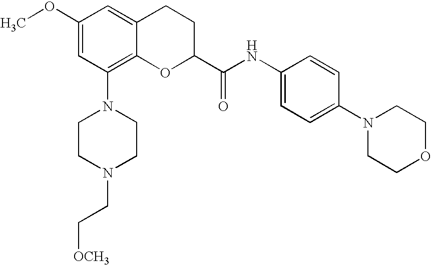 | (+)-6-methoxy-8-(4-(2- methoxyethyl)piperazin-1- yl)-N-(4- morpholinophenyl)chroman- 2-carboxamide (Isomer 1) |
| 73B | 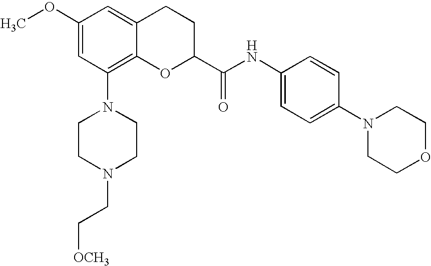 | (−)-6-methoxy-8-(4-(2- methoxyethyl)piperazin-1- yl)-N-(4- morpholinophenyl)chroman- 2-carboxamide (Isomer 2) |
| 75 | 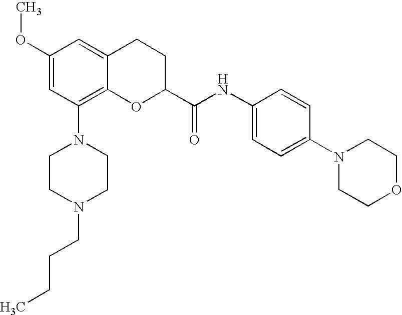 | 8-(4-butylpiperazin-1-yl)-6- methoxy-N-(4- morpholinophenyl)chroman- 2-carboxamide |
| 75A | 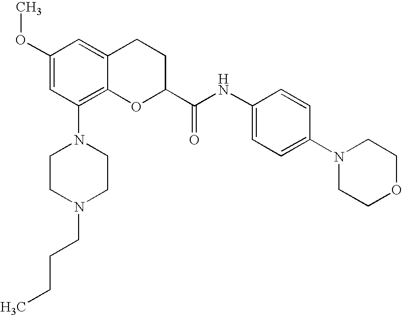 | (+)-8-(4-butylpiperazin-1-yl)- 6-methoxy-N-(4- morpholinophenyl)chroman- 2-carboxamide (Isomer 1) |
| 75B | 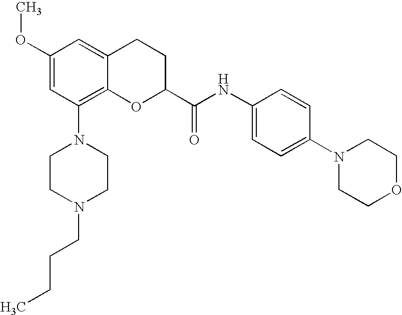 | (−)-8-(4-butylpiperazin-1-yl)- 6-methoxy-N-(4- morpholinophenyl)chroman- 2-carboxamide (Isomer 2) |
| 76 | 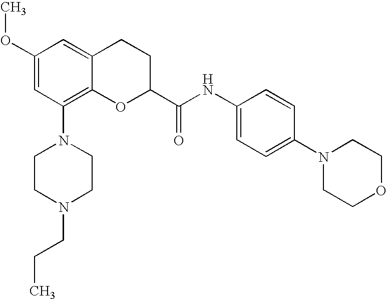 | 6-methoxy-N-(4- morpholinophenyl)-8-(4- propylpiperazin-1- yl)chroman-2-carboxamide |
| 76A | 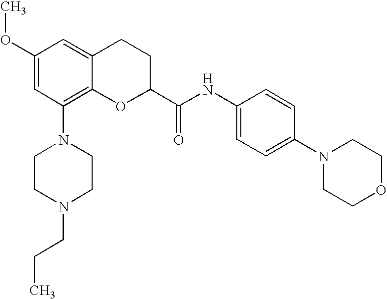 | (+)-6-methoxy-N-(4- morpholinophenyl)-8-(4- propylpiperazin-1- yl)chroman-2-carboxamide (Isomer 1) |
| 76B | 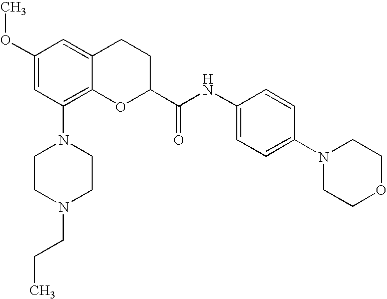 | (−)-6-methoxy-N-(4- morpholinophenyl)-8-(4- propylpiperazin-1- yl)chroman-2-carboxamide (Isomer 2) |
| 77 | 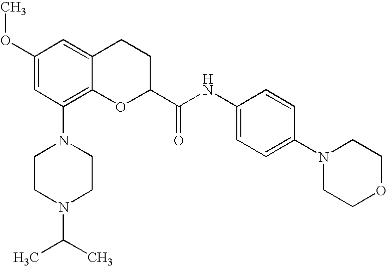 | 8-(4-isopropylpiperazin-1- yl)-6-methoxy-N-(4- morpholinophenyl)chroman- 2-carboxamide |
| 77A | 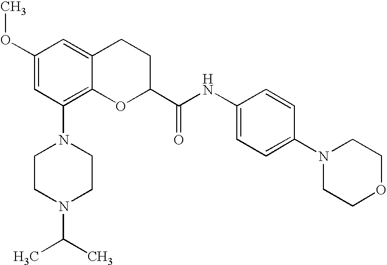 | (+)-8-(4-isopropylpiperazin- 1-yl)-6-methoxy-N-(4- morpholinophenyl)chroman- 2-carboxamide (Isomer 1) |
| 77B | 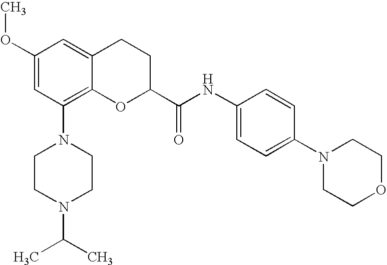 | (−)-8-(4-isopropylpiperazin- 1-yl)-6-methoxy-N-(4- morpholinophenyl)chroman- 2-carboxamide (Isomer 2) |
| 78 | 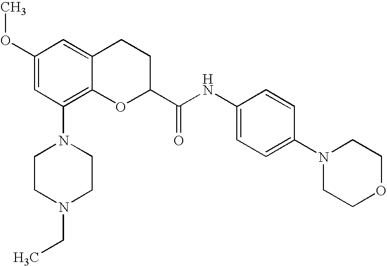 | 8-(4-ethylpiperazin-1-yl)-6- methoxy-N-(4- morpholinophenyl)chroman- 2-carboxamide |
| 78A | 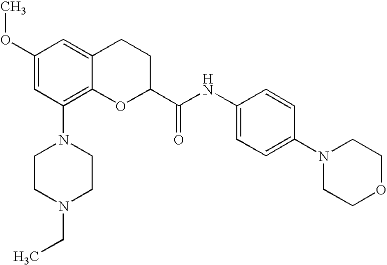 | (+)-8-(4-ethylpiperazin-1-yl)- 6-methoxy-N-(4- morpholinophenyl)chroman- 2-carboxamide (Isomer 1) |
| 78B | 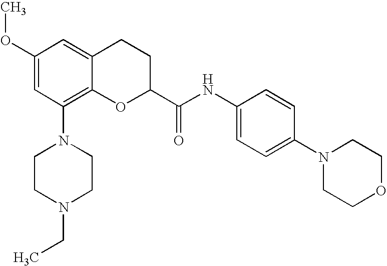 | (−)-8-(4-ethylpiperazin-1-yl)- 6-methoxy-N-(4- morpholinophenyl)chroman- 2-carboxamide (Isomer 2) |
| 79 | 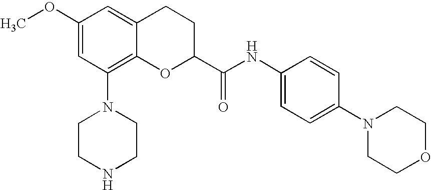 | 6-methoxy-N-(4- morpholinophenyl)-8- (piperazin-1-yl)chroman-2- carboxamide |
| 79C | 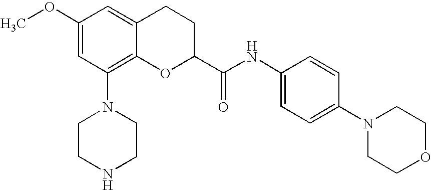 | (+)-6-methoxy-N-(4- morpholinophenyl)-8- (piperazin-1-yl)chroman-2- carboxamide (Isomer 1) |
| 79D | 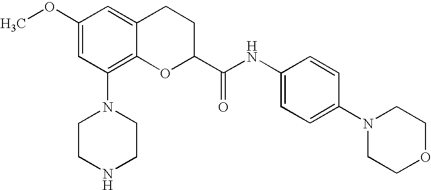 | (−)-6-methoxy-N-(4- morpholinophenyl)-8- (piperazin-1-yl)chroman-2- carboxamide (Isomer 2) |
| 82 | 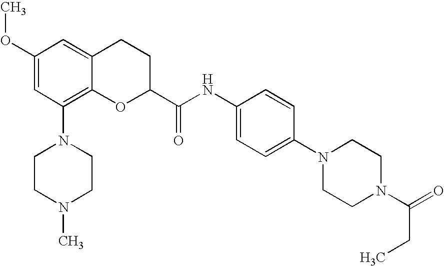 | 6-methoxy-8-(4- methylpiperazin-1-yl)-N-(4- (4-propanoylpiperazin-1- yl)phenyl)chroman-2- carboxamide |
| 82A | 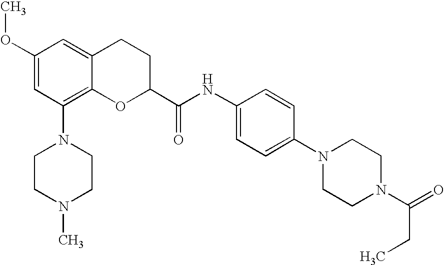 | (+)-6-methoxy-8-(4- methylpiperazin-1-yl)-N-(4- (4-propanoylpiperazin-1- yl)phenyl)chroman-2- carboxamide (Isomer 1) |
| 82B | 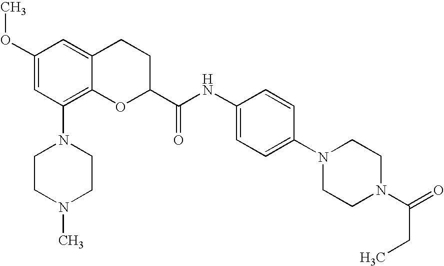 | (−)-6-methoxy-8-(4- methylpiperazin-1-yl)-N-(4- (4-propanoylpiperazin-1- yl)phenyl)chroman-2- carboxamide (Isomer 2) |
| 83 | 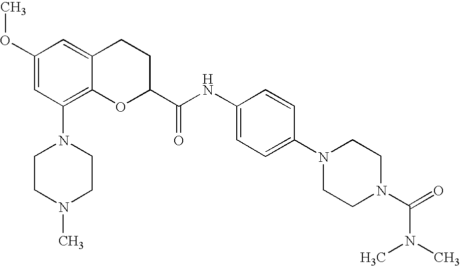 | 4-(4-(6-methoxy-8-(4- methylpiperazin-1- yl)chroman-2- carboxamido)phenyl)-N,N- dimethylpiperazine-1- carboxamide |
| 83A | 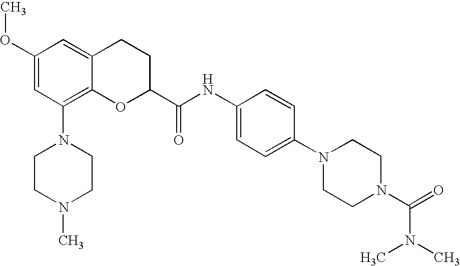 | (+)-4-(4-(6-methoxy-8-(4- methylpiperazin-1- yl)chroman-2- carboxamido)phenyl)-N,N- dimethylpiperazine-1- carboxamide (Isomer 1) |
| 83B | 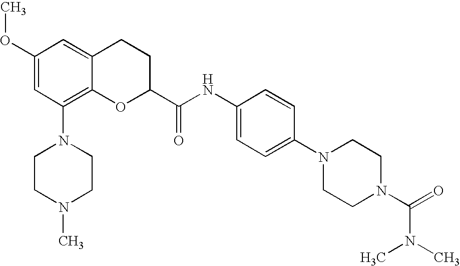 | (−)-4-(4-(6-methoxy-8-(4- methylpiperazin-1- yl)chroman-2- carboxamido)phenyl)-N,N- dimethylpiperazine-1- carboxamide (Isomer 2) |
| 89 | 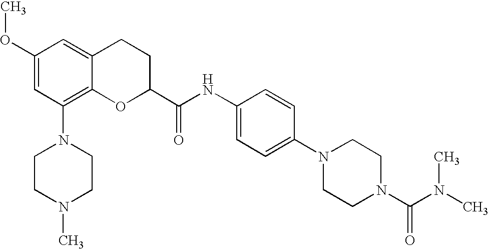 | 4-(4-(6-methoxy-8-(4- methylpiperazin-1- yl)chroman-2- carboxamido)phenyl)-N,N- dimethylpiperazine-1- carboxamide |
| 89A | 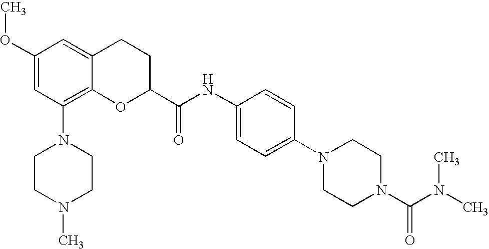 | (+)-4-(4-(6-methoxy-8-(4- methylpiperazin-1- yl)chroman-2- carboxamido)phenyl)-N,N- dimethylpiperazine-1- carboxamide (Isomer 1) |
| 89B | 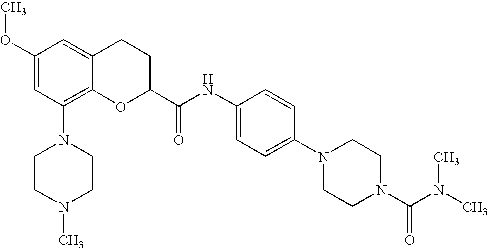 | (−)-4-(4-(6-methoxy-8-(4- methylpiperazin-1- yl)chroman-2- carboxamido)phenyl)-N,N- dimethylpiperazine-1- carboxamide (Isomer 2) |
| 90 | 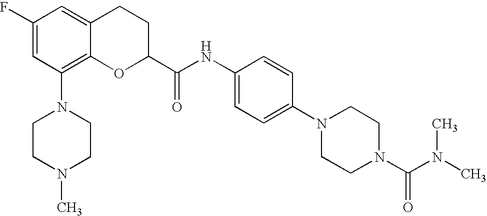 | 4-(4-(6-fluoro-8-(4- methylpiperazin-1- yl)chroman-2- carboxamido)phenyl)-N,N- dimethylpiperazine-1- carboxamide |
| 90A | 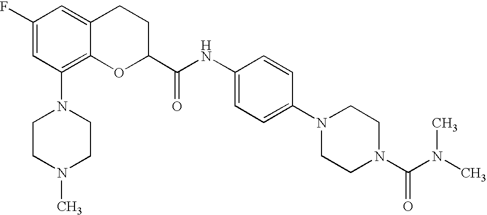 | (+)-4-(4-(6-fluoro-8-(4- methylpiperazin-1- yl)chroman-2- carboxamido)phenyl)-N,N- dimethylpiperazine-1- carboxamide (Isomer 1) |
| 90B | 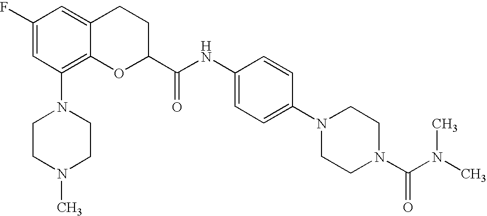 | (−)-4-(4-(6-fluoro-8-(4- methylpiperazin-1- yl)chroman-2- carboxamido)phenyl)-N,N- dimethylpiperazine-1- carboxamide (Isomer 2) |
| 93 | 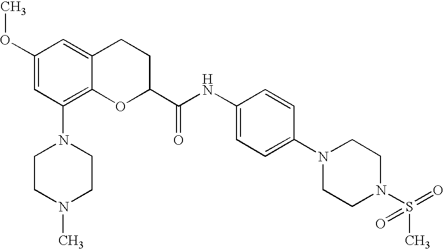 | 6-methoxy-8-(4- methylpiperazin-1-yl)-N-(4- (4-(methylsulfonyl)piperazin- 1-yl)phenyl)chroman-2- carboxamide |
| 93A | 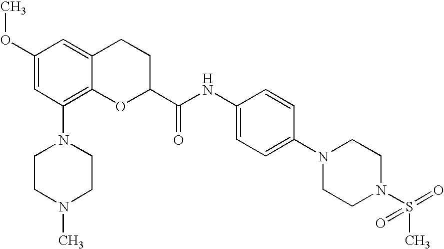 | (+)-6-methoxy-8-(4- methylpiperazin-1-yl)-N-(4- (4-(methylsulfonyl)piperazin- 1-yl)phenyl)chroman-2- carboxamide (Isomer 1) |
| 93B | 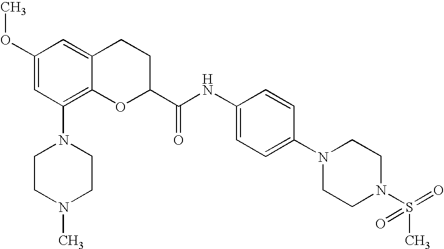 | (−)-6-methoxy-8-(4- methylpiperazin-1-yl)-N-(4- (4-(methylsulfonyl)piperazin- 1-yl)phenyl)chroman-2- carboxamide (Isomer 2) |
| 95 | 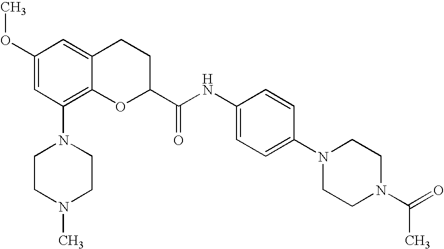 | N-(4-(4-ethanoylpiperazin-1- yl)phenyl)-6-methoxy-8-(4- methylpiperazin-1- yl)chroman-2-carboxamide |
| 95A | 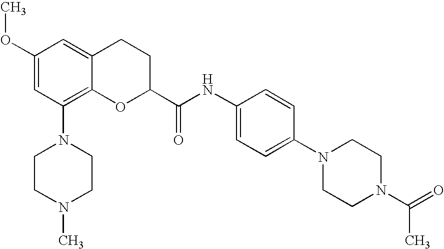 | (+)-N-(4-(4- ethanoylpiperazin-1- yl)phenyl)-6-methoxy-8-(4- methylpiperazin-1- yl)chroman-2-carboxamide (Isomer 1) |
| 95B | 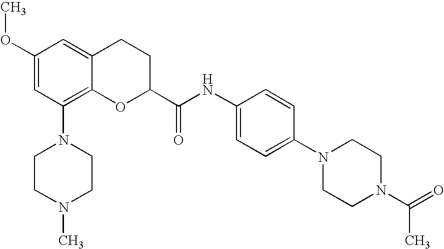 | (−)-N-(4-(4- ethanoylpiperazin-1- yl)phenyl)-6-methoxy-8-(4- methylpiperazin-1- yl)chroman-2-carboxamide (Isomer 2) |
| 96 | 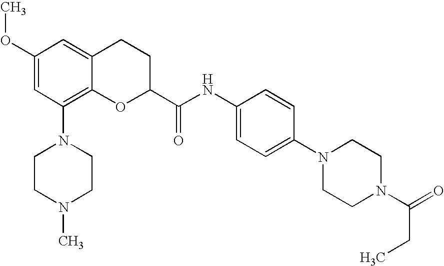 | 6-methoxy-8-(4- methylpiperazin-1-yl)-N-(4- (4-propanoylpiperazin-1- yl)phenyl)chroman-2- carboxamide |
| 96A | 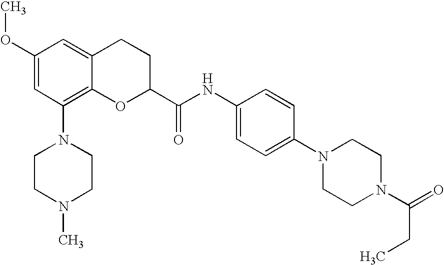 | (+) 6-methoxy-8-(4- methylpiperazin-1-yl)-N-(4- (4-propanoylpiperazin-1- yl)phenyl)chroman-2- carboxamide (Isomer 1) |
| 96B | 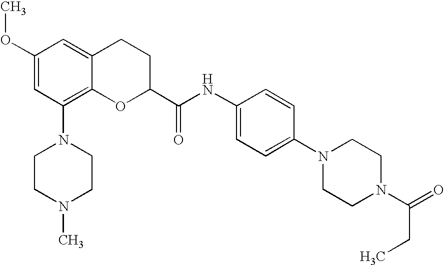 | (−)-6-methoxy-8-(4- methylpiperazin-1-yl)-N-(4- (4-propanoylpiperazin-1- yl)phenyl)chroman-2- carboxamide (Isomer 2) |
| 103 | 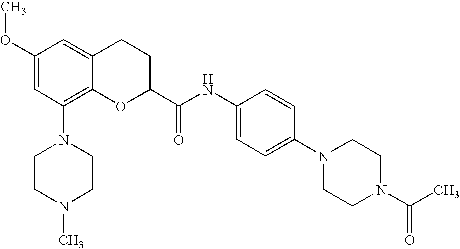 | N-(4-(4-ethanoylpiperazin-1- yl)phenyl)-6-methoxy-8-(4- methylpiperazin-1- yl)chroman-2-carboxamide |
| 103A | 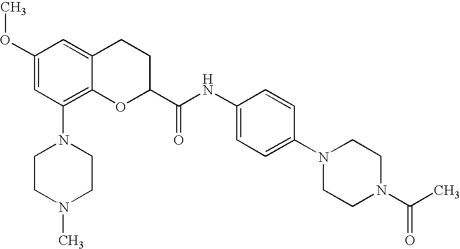 | (+)-N-(4-(4- ethanoylpiperazin-1- yl)phenyl)-6-methoxy-8-(4- methylpiperazin-1- yl)chroman-2-carboxamide (Isomer 1) |
| 103B | 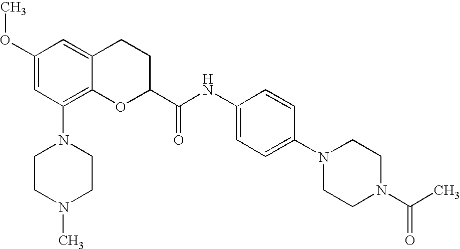 | (−)-N-(4-(4- ethanoylpiperazin-1- yl)phenyl)-6-methoxy-8-(4- methylpiperazin-1- yl)chroman-2-carboxamide (Isomer 2) |

We claim:

1. A compound according to Formula I: 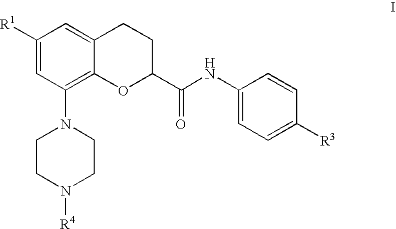
wherein:
R¹ is F or OCH₃;
R⁴is H, CH₃, C₂₋₄alkyl, CH₂CH₂OCH₃, or CH₂CF₃; and
R³is selected from morpholine or piperazine attached by nitrogen and optionally substituted on the other nitrogen (for piperazine) with —C(═O)CH₃, —CH₃, —C(═O)CH₂CH₃, —C(═O)N(CH₃)₂, —SO₂CH₂CH₃, or —SO₂CH₃;
with the proviso that when R¹ is F and R⁴ is methyl then R³ cannot be morpholine, and
with the proviso that when R¹ is F and R⁴ is methyl then R³ cannot be piperazine substituted with —SO₂CH₃, —SO₂CH₂CH₃;
or a pharmaceutically-acceptable salt thereof.

2. A compound according to claim 1 wherein R¹ is F;
or a pharmaceutically-acceptable salt thereof.

3. A compound according to claim 2 wherein R⁴ is CH₃, or C₂₋₄alkyl;
or a pharmaceutically-acceptable salt thereof.

4. A compound according to claim 2 wherein R⁴is CH₃, —CH₂CH₃, —CH₂CH₂CH₃, or —CH(CH₃)₂;
or a pharmaceutically-acceptable salt thereof.

5. A compound according to claim 2 wherein R⁴is —CH₂CH₃, —CH₂CH₂CH₃, or —CH(CH₃)₂;
or a pharmaceutically-acceptable salt thereof.

6. A compound according to claim 2 wherein R⁴ is CH₃;
or a pharmaceutically-acceptable salt thereof.

7. A compound according to claim 2 wherein R³is morpholine,
with the proviso that R⁴ is not methyl;
or a pharmaceutically-acceptable salt thereof.

8. A compound according to claim 2 wherein R³ is piperazine attached by nitrogen and optionally substituted on the other nitrogen with —C(═O)CH₃, —CH₃, —C(═O)CH₂CH₃, —C(═O)N(CH₃)₂, —SO₂CH₂CH₃, or —SO₂CH₃, or a pharmaceutically-acceptable salt thereof.

9. A compound according to claim 2 wherein R³is piperazine attached by nitrogen and optionally substituted on the other nitrogen with —C(═O)CH₃, —CH₃, —C(═O)CH₂CH₃, or —C(═O)N(CH₃)₂ or a pharmaceutically-acceptable salt thereof.

10. A compound according to claim 2 wherein R³is piperazine attached by nitrogen and optionally substituted on the other nitrogen with —C(═O)CH₃, or —C(═O)CH₂CH₃ or a pharmaceutically-acceptable salt thereof.

11. A compound according to claim 2 wherein R³is piperazine attached by nitrogen and optionally substituted on the other nitrogen with —C(═O)CH₃ or a pharmaceutically-acceptable salt thereof.

12. A compound according to claim 1 wherein R¹ is OCH₃;
or a pharmaceutically-acceptable salt thereof.

13. A compound according to claim 12 wherein R⁴is CH₃, or C₂₋₄alkyl;
or a pharmaceutically-acceptable salt thereof.

14. A compound according to claim 12 wherein R⁴is CH₃, —CH₂CH₃, —CH₂CH₂CH₃, or —CH(CH₃)₂;
or a pharmaceutically-acceptable salt thereof.

15. A compound according to claim 12 wherein R⁴is —CH₂CH₃, —CH₂CH₂CH₃, or —CH(CH₃)₂;
or a pharmaceutically-acceptable salt thereof.

16. A compound according to claim 12 wherein R⁴is CH₃;
or a pharmaceutically-acceptable salt thereof.

17. A compound according to claim 12 wherein R³ is morpholine,
or a pharmaceutically-acceptable salt thereof.

18. A compound according to claim 12 wherein R³is piperazine attached by nitrogen and optionally substituted on the other nitrogen with —C(═O)CH₃, —CH₃, —C(═O)CH₂CH₃, —C(═O)N(CH₃)₂, —SO₂CH₂CH₃, or —SO₂CH₃, or a pharmaceutically-acceptable salt thereof.

19. A compound according to claim 12 wherein R³is piperazine attached by nitrogen and optionally substituted on the other nitrogen with —C(═O)CH₃, —CH₃, —C(═O)CH₂CH₃, or —C(═O)N(CH₃)₂ or a pharmaceutically-acceptable salt thereof.

20. A compound according to claim 12 wherein R³ is piperazine attached by nitrogen and optionally substituted on the other nitrogen with —C(═O)CH₃, or —C(═O)CH₂CH₃ or a pharmaceutically-acceptable salt thereof.

21. A compound according to claim 12 wherein R³is piperazine attached by nitrogen and optionally substituted on the other nitrogen with —C(═O)CH₃;
or a pharmaceutically-acceptable salt thereof.

22. A compound according to claim 1 wherein:
R¹ is F;
R⁴ is CH₃, or —CH₂CH₃; and
R³ is piperazine attached by nitrogen and optionally substituted on the other nitrogen with —C(═O)CH₃ or a pharmaceutically-acceptable salt thereof.

23. A compound according to claim 1 wherein:
R¹ is OCH₃;
R⁴ is CH₃, or —CH₂CH₃; and
R³ is morpholine;
or a pharmaceutically-acceptable salt thereof.

24. A compound selected from:
6-methoxy-8-(4-methylpiperazin-1-yl)-N-(4-morpholinophenyl)chroman-2-carboxamide;
(+)-6-methoxy-8-(4-methylpiperazin-1-yl)-N-(4-morpholinophenyl)chroman-2-carboxamide;
(−)-6-methoxy-8-(4-methylpiperazin-1-yl)-N-(4-morpholinophenyl)chroman-2-carboxamide;
N-(4-(4-acetylpiperazin-1-yl)phenyl)-6-fluoro-8-(4-methylpiperazin-1-yl)chroman-2-carboxamide;
(+)-N-(4-(4-acetylpiperazin-1-yl)phenyl)-6-fluoro-8-(4-methylpiperazin-1-yl)chroman-2-carboxamide;
(−)-N-(4-(4-acetylpiperazin-1-yl)phenyl)-6-fluoro-8-(4-methylpiperazin-1-yl)chroman-2-carboxamide;
N-(4-(4-ethanoylpiperazin-1-yl)phenyl)-6-fluoro-8-(4-methylpiperazin-1-yl)chroman-2-carboxamide;
(+)-N-(4-(4-ethanoylpiperazin-1-yl)phenyl)-6-fluoro-8-(4-methylpiperazin-1-yl)chroman-2-carboxamide;
(−)-N-(4-(4-ethanoylpiperazin-1-yl)phenyl)-6-fluoro-8-(4-methylpiperazin-1-yl)chroman-2-carboxamide;
6-fluoro-8-(4-methylpiperazin-1-yl)-N-(4-(4-propanoylpiperazin-1-yl)phenyl)chroman-2-carboxamide;
(+)-6-fluoro-8-(4-methylpiperazin-1-yl)-N-(4-(4-propanoylpiperazin-1-yl)phenyl)chroman-2-carboxamide;
(−)-6-fluoro-8-(4-methylpiperazin-1-yl)-N-(4-(4-propanoylpiperazin-1-yl)phenyl)chroman-2-carboxamide;
6-methoxy-8-(4-methylpiperazin-1-yl)-N-(4-(4-methylpiperazin-1-yl)phenyl)chroman-2-carboxamide;
(+)-6-methoxy-8-(4-methylpiperazin-1-yl)-N-(4-(4-methylpiperazin-1-yl)phenyl)chroman-2-carboxamide;
(−)-6-methoxy-8-(4-methylpiperazin-1-yl)-N-(4-(4-methylpiperazin-1-yl)phenyl)chroman-2-carboxamide;
6-fluoro-8-(4-methylpiperazin-1-yl)-N-(4-(4-methylpiperazin-1-yl)phenyl)chroman-2-carboxamide;
(+)-6-fluoro-8-(4-methylpiperazin-1-yl)-N-(4-(4-methylpiperazin-1-yl)phenyl)chroman-2-carboxamide;
(−)-6-fluoro-8-(4-methylpiperazin-1-yl)-N-(4-(4-methylpiperazin-1-yl)phenyl)chroman-2-carboxamide;
6-fluoro-8-(4-methylpiperazin-1-yl)-N-(4-(4-propanoylpiperazin-1-yl)phenyl)chroman-2-carboxamide;
(+)-6-fluoro-8-(4-methylpiperazin-1-yl)-N-(4-(4-propanoylpiperazin-1-yl)phenyl)chroman-2-carboxamide;
(−)-6-fluoro-8-(4-methylpiperazin-1-yl)-N-(4-(4-propanoylpiperazin-1-yl)phenyl)chroman-2-carboxamide;
N-(4-(4-(ethylsulfonyl)piperazin-1-yl)phenyl)-6-methoxy-8-(4-methylpiperazin-1-yl)chroman-2-carboxamide;
(+)-N-(4-(4-(ethylsulfonyl)piperazin-1-yl)phenyl)-6-methoxy-8-(4-methylpiperazin-1-yl)chroman-2-carboxamide;
(−)-N-(4-(4-(ethylsulfonyl)piperazin-1-yl)phenyl)-6-methoxy-8-(4-methylpiperazin-1-yl)chroman-2-carboxamide;
6-methoxy-N-(4-morpholinophenyl)-8-(4-(2,2,2-trifluoroethyl)piperazin-1-yl)chroman-2-carboxamide;
(+)-6-methoxy-N-(4-morpholinophenyl)-8-(4-(2,2,2-trifluoroethyl)piperazin-1-yl)chroman-2-carboxamide;
(−)-6-methoxy-N-(4-morpholinophenyl)-8-(4-(2,2,2-trifluoroethyl)piperazin-1-yl)chroman-2-carboxamide;
6-methoxy-8-(4-(2-methoxyethyl)piperazin-1-yl)-N-(4-morpholinophenyl)chroman-2-carboxamide;
(+)-6-methoxy-8-(4-(2-methoxyethyl)piperazin-1-yl)-N-(4-morpholinophenyl)chroman-2-carboxamide;
(−)-6-methoxy-8-(4-(2-methoxyethyl)piperazin-1-yl)-N-(4-morpholinophenyl)chroman-2-carboxamide;
8-(4-butylpiperazin-1-yl)-6-methoxy-N-(4-morpholinophenyl)chroman-2-carboxamide;
(+)-8-(4-butylpiperazin-1-yl)-6-methoxy-N-(4-morpholinophenyl)chroman-2-carboxamide;
(−)-8-(4-butylpiperazin-1-yl)-6-methoxy-N-(4-morpholinophenyl)chroman-2-carboxamide;
6-methoxy-N-(4-morpholinophenyl)-8-(4-propylpiperazin-1-yl)chroman-2-carboxamide;
(+)-6-methoxy-N-(4-morpholinophenyl)-8-(4-propylpiperazin-1-yl)chroman-2-carboxamide;
(−)-6-methoxy-N-(4-morpholinophenyl)-8-(4-propylpiperazin-1-yl)chroman-2-carboxamide;
8-(4-isopropylpiperazin-1-yl)-6-methoxy-N-(4-morpholinophenyl)chroman-2-carboxamide;
(+)-8-(4-isopropylpiperazin-1-yl)-6-methoxy-N-(4-morpholinophenyl)chroman-2-carboxamide;
(−)-8-(4-isopropylpiperazin-1-yl)-6-methoxy-N-(4-morpholinophenyl)chroman-2-carboxamide;
8-(4-ethylpiperazin-1-yl)-6-methoxy-N-(4-morpholinophenyl)chroman-2-carboxamide;
(+)-8-(4-ethylpiperazin-1-yl)-6-methoxy-N-(4-morpholinophenyl)chroman-2-carboxamide;
(−)-8-(4-ethylpiperazin-1-yl)-6-methoxy-N-(4-morpholinophenyl)chroman-2-carboxamide;
6-methoxy-N-(4-morpholinophenyl)-8-(piperazin-1-yl)chroman-2-carboxamide;
(+)-6-methoxy-N-(4-morpholinophenyl)-8-(piperazin-1-yl)chroman-2-carboxamide;
(−)-6-methoxy-N-(4-morpholinophenyl)-8-(piperazin-1-yl)chroman-2-carboxamide;
6-methoxy-8-(4-methylpiperazin-1-yl)-N-(4-(4-propanoylpiperazin-1-yl)phenyl)chroman-2-carboxamide;
(+)-6-methoxy-8-(4-methylpiperazin-1-yl)-N-(4-(4-propanoylpiperazin-1-yl)phenyl)chroman-2-carboxamide;
(−)-6-methoxy-8-(4-methylpiperazin-1-yl)-N-(4-(4-propanoylpiperazin-1-yl)phenyl)chroman-2-carboxamide;
4-(4-(6-methoxy-8-(4-methylpiperazin-1-yl)chroman-2-carboxamido)phenyl)-N,N-dimethylpiperazine-1-carboxamide;
(+)-4-(4-(6-methoxy-8-(4-methylpiperazin-1-yl)chroman-2-carboxamido)phenyl)-N,N-dimethylpiperazine-1-carboxamide;
(−)-4-(4-(6-methoxy-8-(4-methylpiperazin-1-yl)chroman-2-carboxamido)phenyl)-N,N-dimethylpiperazine-1-carboxamide;
4-(4-(6-methoxy-8-(4-methylpiperazin-1-yl)chroman-2-carboxamido)phenyl)-N,N-dimethylpiperazine-1-carboxamide;
(+)-4-(4-(6-methoxy-8-(4-methylpiperazin-1-yl)chroman-2-carboxamido)phenyl)-N,N-dimethylpiperazine-1-carboxamide;
(−)-4-(4-(6-methoxy-8-(4-methylpiperazin-1-yl)chroman-2-carboxamido)phenyl)-N,N-dimethylpiperazine-1-carboxamide;
4-(4-(6-fluoro-8-(4-methylpiperazin-1-yl)chroman-2-carboxamido)phenyl)-N,N-dimethylpiperazine-1-carboxamide;
(+)-4-(4-(6-fluoro-8-(4-methylpiperazin-1-yl)chroman-2-carboxamido)phenyl)-N,N-dimethylpiperazine-1-carboxamide;
(−)-4-(4-(6-fluoro-8-(4-methylpiperazin-1-yl)chroman-2-carboxamido)phenyl)-N,N-dimethylpiperazine-1-carboxamide;
6-methoxy-8-(4-methylpiperazin-1-yl)-N-(4-(4-(methylsulfonyl)piperazin-1-yl)phenyl)chroman-2-carboxamide;
(+)-6-methoxy-8-(4-methylpiperazin-1-yl)-N-(4-(4-(methylsulfonyl)piperazin-1-yl)phenyl)chroman-2-carboxamide;
(−)-6-methoxy-8-(4-methylpiperazin-1-yl)-N-(4-(4-(methylsulfonyl)piperazin-1-yl)phenyl)chroman-2-carboxamide;
N-(4-(4-ethanoylpiperazin-1-yl)phenyl)-6-methoxy-8-(4-methylpiperazin-1-yl)chroman-2-carboxamide;
(+)-N-(4-(4-ethanoylpiperazin-1-yl)phenyl)-6-methoxy-8-(4-methylpiperazin-1-yl)chroman-2-carboxamide;
(−)-N-(4-(4-ethanoylpiperazin-1-yl)phenyl)-6-methoxy-8-(4-methylpiperazin-1-yl)chroman-2-carboxamide;
6-methoxy-8-(4-methylpiperazin-1-yl)-N-(4-(4-propanoylpiperazin-1-yl)phenyl)chroman-2-carboxamide;
(+) 6-methoxy-8-(4-methylpiperazin-1-yl)-N-(4-(4-propanoylpiperazin-1-yl)phenyl)chroman-2-carboxamide;
(−)-6-methoxy-8-(4-methylpiperazin-1-yl)-N-(4-(4-propanoylpiperazin-1-yl)phenyl)chroman-2-carboxamide;
N-(4-(4-ethanoylpiperazin-1-yl)phenyl)-6-methoxy-8-(4-methylpiperazin-1-yl)chroman-2-carboxamide;
(+)-N-(4-(4-ethanoylpiperazin-1-yl)phenyl)-6-methoxy-8-(4-methylpiperazin-1-yl)chroman-2-carboxamide; and
(−)-N-(4-(4-ethanoylpiperazin-1-yl)phenyl)-6-methoxy-8-(4-methylpiperazin-1-yl)chroman-2-carboxamide;
or a pharmaceutically-acceptable salt thereof.

25. A compound according to claim 1, wherein said compound is dextrorotatory, or a pharmaceutically-acceptable salt thereof.

26. A compound selected from:
(+)-6-methoxy-8-(4-methylpiperazin-1-yl)-N-(4-morpholinophenyl)chroman-2-carboxamide;
(+)-N-(4-(4-ethanoylpiperazin-1-yl)phenyl)-6-fluoro-8-(4-methylpiperazin-1-yl)chroman-2-carboxamide;
(+)-N-(4-(4-ethanoylpiperazin-1-yl)phenyl)-6-fluoro-8-(4-methylpiperazin-1-yl)chroman-2-carboxamide;
(+)-6-fluoro-8-(4-methylpiperazin-1-yl)-N-(4-(4-propanoylpiperazin-1-yl)phenyl)chroman-2-carboxamide;
(+)-6-methoxy-8-(4-methylpiperazin-1-yl)-N-(4-(4-methylpiperazin-1-yl)phenyl)chroman-2-carboxamide;
(+)-6-fluoro-8-(4-methylpiperazin-1-yl)-N-(4-(4-methylpiperazin-1-yl)phenyl)chroman-2-carboxamide;
(+)-6-fluoro-8-(4-methylpiperazin-1-yl)-N-(4-(4-propanoylpiperazin-1-yl)phenyl)chroman-2-carboxamide;
(+)-N-(4-(4-(ethylsulfonyl)piperazin-1-yl)phenyl)-6-methoxy-8-(4-methylpiperazin-1-yl)chroman-2-carboxamide
(+)-8-(4-butylpiperazin-1-yl)-6-methoxy-N-(4-morpholinophenyl)chroman-2-carboxamide;
(+)-6-methoxy-N-(4-morpholinophenyl)-8-(4-propylpiperazin-1-yl)chroman-2-carboxamide;
(+)-8-(4-isopropylpiperazin-1-yl)-6-methoxy-N-(4-morpholinophenyl)chroman-2-carboxamide;
(+)-8-(4-ethylpiperazin-1-yl)-6-methoxy-N-(4-morpholinophenyl)chroman-2-carboxamide;
(+)-6-methoxy-8-(4-methylpiperazin-1-yl)-N-(4-(4-propanoylpiperazin-1-yl)phenyl)chroman-2-carboxamide;
(+)-4-(4-(6-methoxy-8-(4-methylpiperazin-1-yl)chroman-2-carboxamido)phenyl)-N,N-dimethylpiperazine-1-carboxamide;
(+)-4-(4-(6-methoxy-8-(4-methylpiperazin-1-yl)chroman-2-carboxamido)phenyl)-N,N-dimethylpiperazine-1-carboxamide;
(+)-4-(4-(6-fluoro-8-(4-methylpiperazin-1-yl)chroman-2-carboxamido)phenyl)-N,N-dimethylpiperazine-1-carboxamide;
(+)-6-methoxy-8-(4-methylpiperazin-1-yl)-N-(4-(4-(methylsulfonyl)piperazin-1-yl)phenyl)chroman-2-carboxamide;
(+)-N-(4-(4-ethanoylpiperazin-1-yl)phenyl)-6-methoxy-8-(4-methylpiperazin-1-yl)chroman-2-carboxamide;
(+) 6-methoxy-8-(4-methylpiperazin-1-yl)-N-(4-(4-propanoylpiperazin-1-yl)phenyl)chroman-2-carboxamide; and
(+)-N-(4-(4-ethanoylpiperazin-1-yl)phenyl)-6-methoxy-8-(4-methylpiperazin-1-yl)chroman-2-carboxamide;
or a pharmaceutically-acceptable salt thereof.

27. A compound according to claim 1 for use in the treatment of anxiety disorders or mood disorders or cognitive disorders in a mammal.

28. A method of treating anxiety disorders in a mammal comprising administering to such mammal an effective amount of a compound of formula I 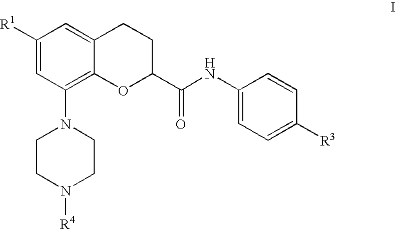
wherein:
R¹ is F or OCH₃;
R⁴ is CH₃, or C₂₋₄alkyl; and
R³ is selected from morpholine or piperazine attached by nitrogen and optionally substituted on the other nitrogen (for piperazine) with —C(═O)CH₃, —CH₃, —C(═O)CH₂CH₃, —C(═O)N(CH₃)₂, —SO₂CH₂CH₃, or —SO₂CH₃,
with the proviso that when R¹ is F and R⁴ is methyl then R³ cannot be morpholine; and
with the proviso that when R¹ is F and R⁴ is methyl then R³ cannot be piperazine substituted with —CH₂OH, —CH₂CH₂OH, —SO₂CH₃, or —SO₂CH₂CH₃;
or a pharmaceutically-acceptable salt thereof.

29. The method of claim 28 wherein the compound is selected from:
6-methoxy-8-(4-methylpiperazin-1-yl)-N-(4-morpholinophenyl)chroman-2-carboxamide;
(+)-6-methoxy-8-(4-methylpiperazin-1-yl)-N-(4-morpholinophenyl)chroman-2-carboxamide;
(−)-6-methoxy-8-(4-methylpiperazin-1-yl)-N-(4-morpholinophenyl)chroman-2-carboxamide;
N-(4-(4-ethanoylpiperazin-1-yl)phenyl)-6-fluoro-8-(4-methylpiperazin-1-yl)chroman-2-carboxamide;
(+)-N-(4-(4-ethanoylpiperazin-1-yl)phenyl)-6-fluoro-8-(4-methylpiperazin-1-yl)chroman-2-carboxamide;
(−)-N-(4-(4-ethanoylpiperazin-1-yl)phenyl)-6-fluoro-8-(4-methylpiperazin-1-yl)chroman-2-carboxamide;
N-(4-(4-ethanoylpiperazin-1-yl)phenyl)-6-fluoro-8-(4-methylpiperazin-1-yl)chroman-2-carboxamide;
(+)-N-(4-(4-ethanoylpiperazin-1-yl)phenyl)-6-fluoro-8-(4-methylpiperazin-1-yl)chroman-2-carboxamide;
(−)-N-(4-(4-ethanoylpiperazin-1-yl)phenyl)-6-fluoro-8-(4-methylpiperazin-1-yl)chroman-2-carboxamide;
6-fluoro-8-(4-methylpiperazin-1-yl)-N-(4-(4-propanoylpiperazin-1-yl)phenyl)chroman-2-carboxamide;
(+)-6-fluoro-8-(4-methylpiperazin-1-yl)-N-(4-(4-propanoylpiperazin-1-yl)phenyl)chroman-2-carboxamide;
(−)-6-fluoro-8-(4-methylpiperazin-1-yl)-N-(4-(4-propanoylpiperazin-1-yl)phenyl)chroman-2-carboxamide;
6-methoxy-8-(4-methylpiperazin-1-yl)-N-(4-(4-methylpiperazin-1-yl)phenyl)chroman-2-carboxamide;
(+)-6-methoxy-8-(4-methylpiperazin-1-yl)-N-(4-(4-methylpiperazin-1-yl)phenyl)chroman-2-carboxamide;
(−)-6-methoxy-8-(4-methylpiperazin-1-yl)-N-(4-(4-methylpiperazin-1-yl)phenyl)chroman-2-carboxamide;
6-fluoro-8-(4-methylpiperazin-1-yl)-N-(4-(4-methylpiperazin-1-yl)phenyl)chroman-2-carboxamide;
(+)-6-fluoro-8-(4-methylpiperazin-1-yl)-N-(4-(4-methylpiperazin-1-yl)phenyl)chroman-2-carboxamide;
(−)-6-fluoro-8-(4-methylpiperazin-1-yl)-N-(4-(4-methylpiperazin-1-yl)phenyl)chroman-2-carboxamide;
6-fluoro-8-(4-methylpiperazin-1-yl)-N-(4-(4-propanoylpiperazin-1-yl)phenyl)chroman-2-carboxamide;
(+)-6-fluoro-8-(4-methylpiperazin-1-yl)-N-(4-(4-propanoylpiperazin-1-yl)phenyl)chroman-2-carboxamide;
(−)-6-fluoro-8-(4-methylpiperazin-1-yl)-N-(4-(4-propanoylpiperazin-1-yl)phenyl)chroman-2-carboxamide;
N-(4-(4-(ethylsulfonyl)piperazin-1-yl)phenyl)-6-methoxy-8-(4-methylpiperazin-1-yl)chroman-2-carboxamide;
(+)-N-(4-(4-(ethylsulfonyl)piperazin-1-yl)phenyl)-6-methoxy-8-(4-methylpiperazin-1-yl)chroman-2-carboxamide;
(−)-N-(4-(4-(ethylsulfonyl)piperazin-1-yl)phenyl)-6-methoxy-8-(4-methylpiperazin-1-yl)chroman-2-carboxamide;
8-(4-butylpiperazin-1-yl)-6-methoxy-N-(4-morpholinophenyl)chroman-2-carboxamide;
(+)-8-(4-butylpiperazin-1-yl)-6-methoxy-N-(4-morpholinophenyl)chroman-2-carboxamide;
(−)-8-(4-butylpiperazin-1-yl)-6-methoxy-N-(4-morpholinophenyl)chroman-2-carboxamide;
6-methoxy-N-(4-morpholinophenyl)-8-(4-propylpiperazin-1-yl)chroman-2-carboxamide;
(+)-6-methoxy-N-(4-morpholinophenyl)-8-(4-propylpiperazin-1-yl)chroman-2-carboxamide;
(−)-6-methoxy-N-(4-morpholinophenyl)-8-(4-propylpiperazin-1-yl)chroman-2-carboxamide;
8-(4-isopropylpiperazin-1-yl)-6-methoxy-N-(4-morpholinophenyl)chroman-2-carboxamide;
(+)-8-(4-isopropylpiperazin-1-yl)-6-methoxy-N-(4-morpholinophenyl)chroman-2-carboxamide;
(−)-8-(4-isopropylpiperazin-1-yl)-6-methoxy-N-(4-morpholinophenyl)chroman-2-carboxamide;
8-(4-ethylpiperazin-1-yl)-6-methoxy-N-(4-morpholinophenyl)chroman-2-carboxamide;
(+)-8-(4-ethylpiperazin-1-yl)-6-methoxy-N-(4-morpholinophenyl)chroman-2-carboxamide;
(−)-8-(4-ethylpiperazin-1-yl)-6-methoxy-N-(4-morpholinophenyl)chroman-2-carboxamide;
6-methoxy-8-(4-methylpiperazin-1-yl)-N-(4-(4-propanoylpiperazin-1-yl)phenyl)chroman-2-carboxamide;
(+)-6-methoxy-8-(4-methylpiperazin-1-yl)-N-(4-(4-propanoylpiperazin-1-yl)phenyl)chroman-2-carboxamide;
(−)-6-methoxy-8-(4-methylpiperazin-1-yl)-N-(4-(4-propanoylpiperazin-1-yl)phenyl)chroman-2-carboxamide;
4-(4-(6-methoxy-8-(4-methylpiperazin-1-yl)chroman-2-carboxamido)phenyl)-N,N-dimethylpiperazine-1-carboxamide;
(+)-4-(4-(6-methoxy-8-(4-methylpiperazin-1-yl)chroman-2-carboxamido)phenyl)-N,N-dimethylpiperazine-1-carboxamide;
(−)-4-(4-(6-methoxy-8-(4-methylpiperazin-1-yl)chroman-2-carboxamido)phenyl)-N,N-dimethylpiperazine-1-carboxamide;
4-(4-(6-methoxy-8-(4-methylpiperazin-1-yl)chroman-2-carboxamido)phenyl)-N,N-dimethylpiperazine-1-carboxamide;
(+)-4-(4-(6-methoxy-8-(4-methylpiperazin-1-yl)chroman-2-carboxamido)phenyl)-N,N-dimethylpiperazine-1-carboxamide;
(−)-4-(4-(6-methoxy-8-(4-methylpiperazin-1-yl)chroman-2-carboxamido)phenyl)-N,N-dimethylpiperazine-1-carboxamide;
4-(4-(6-fluoro-8-(4-methylpiperazin-1-yl)chroman-2-carboxamido)phenyl)-N,N-dimethylpiperazine-1-carboxamide;
(+)-4-(4-(6-fluoro-8-(4-methylpiperazin-1-yl)chroman-2-carboxamido)phenyl)-N,N-dimethylpiperazine-1-carboxamide;
(−)-4-(4-(6-fluoro-8-(4-methylpiperazin-1-yl)chroman-2-carboxamido)phenyl)-N,N-dimethylpiperazine-1-carboxamide;
6-methoxy-8-(4-methylpiperazin-1-yl)-N-(4-(4-(methylsulfonyl)piperazin-1-yl)phenyl)chroman-2-carboxamide;
(+)-6-methoxy-8-(4-methylpiperazin-1-yl)-N-(4-(4-(methylsulfonyl)piperazin-1-yl)phenyl)chroman-2-carboxamide;
(−)-6-methoxy-8-(4-methylpiperazin-1-yl)-N-(4-(4-(methylsulfonyl)piperazin-1-yl)phenyl)chroman-2-carboxamide;
N-(4-(4-ethanoylpiperazin-1-yl)phenyl)-6-methoxy-8-(4-methylpiperazin-1-yl)chroman-2-carboxamide;
(+)-N-(4-(4-ethanoylpiperazin-1-yl)phenyl)-6-methoxy-8-(4-methylpiperazin-1-yl)chroman-2-carboxamide;
(−)-N-(4-(4-ethanoylpiperazin-1-yl)phenyl)-6-methoxy-8-(4-methylpiperazin-1-yl)chroman-2-carboxamide;
6-methoxy-8-(4-methylpiperazin-1-yl)-N-(4-(4-propanoylpiperazin-1-yl)phenyl)chroman-2-carboxamide;
(+) 6-methoxy-8-(4-methylpiperazin-1-yl)-N-(4-(4-propanoylpiperazin-1-yl)phenyl)chroman-2-carboxamide;
(−)-6-methoxy-8-(4-methylpiperazin-1-yl)-N-(4-(4-propanoylpiperazin-1-yl)phenyl)chroman-2-carboxamide;
N-(4-(4-ethanoylpiperazin-1-yl)phenyl)-6-methoxy-8-(4-methylpiperazin-1-yl)chroman-2-carboxamide;
(+)-N-(4-(4-ethanoylpiperazin-1-yl)phenyl)-6-methoxy-8-(4-methylpiperazin-1-yl)chroman-2-carboxamide; and
(−)-N-(4-(4-ethanoylpiperazin-1-yl)phenyl)-6-methoxy-8-(4-methylpiperazin-1-yl)chroman-2-carboxamide;
or a pharmaceutically-acceptable salt thereof.

30. The method of claim 28 wherein the compound is selected from:
(+)-6-methoxy-8-(4-methylpiperazin-1-yl)-N-(4-morpholinophenyl)chroman-2-carboxamide;
(+)-N-(4-(4-ethanoylpiperazin-1-yl)phenyl)-6-fluoro-8-(4-methylpiperazin-1-yl)chroman-2-carboxamide;
(+)-N-(4-(4-ethanoylpiperazin-1-yl)phenyl)-6-fluoro-8-(4-methylpiperazin-1-yl)chroman-2-carboxamide;
(+)-6-fluoro-8-(4-methylpiperazin-1-yl)-N-(4-(4-propanoylpiperazin-1-yl)phenyl)chroman-2-carboxamide;
(+)-6-methoxy-8-(4-methylpiperazin-1-yl)-N-(4-(4-methylpiperazin-1-yl)phenyl)chroman-2-carboxamide;
(+)-6-fluoro-8-(4-methylpiperazin-1-yl)-N-(4-(4-methylpiperazin-1-yl)phenyl)chroman-2-carboxamide;
(+)-6-fluoro-8-(4-methylpiperazin-1-yl)-N-(4-(4-propanoylpiperazin-1-yl)phenyl)chroman-2-carboxamide;
(+)-N-(4-(4-(ethylsulfonyl)piperazin-1-yl)phenyl)-6-methoxy-8-(4-methylpiperazin-1-yl)chroman-2-carboxamide
(+)-8-(4-butylpiperazin-1-yl)-6-methoxy-N-(4-morpholinophenyl)chroman-2-carboxamide;
(+)-6-methoxy-N-(4-morpholinophenyl)-8-(4-propylpiperazin-1-yl)chroman-2-carboxamide;
(+)-8-(4-isopropylpiperazin-1-yl)-6-methoxy-N-(4-morpholinophenyl)chroman-2-carboxamide;
(+)-8-(4-ethylpiperazin-1-yl)-6-methoxy-N-(4-morpholinophenyl)chroman-2-carboxamide;
(+)-6-methoxy-8-(4-methylpiperazin-1-yl)-N-(4-(4-propanoylpiperazin-1-yl)phenyl)chroman-2-carboxamide;
(+)-4-(4-(6-methoxy-8-(4-methylpiperazin-1-yl)chroman-2-carboxamido)phenyl)-N,N-dimethylpiperazine-1-carboxamide;
(+)-4-(4-(6-methoxy-8-(4-methylpiperazin-1-yl)chroman-2-carboxamido)phenyl)-N,N-dimethylpiperazine-1-carboxamide;
(+)-4-(4-(6-fluoro-8-(4-methylpiperazin-1-yl)chroman-2-carboxamido)phenyl)-N,N-dimethylpiperazine-1-carboxamide;
(+)-6-methoxy-8-(4-methylpiperazin-1-yl)-N-(4-(4-(methylsulfonyl)piperazin-1-yl)phenyl)chroman-2-carboxamide;
(+)-N-(4-(4-ethanoylpiperazin-1-yl)phenyl)-6-methoxy-8-(4-methylpiperazin-1-yl)chroman-2-carboxamide;
(+) 6-methoxy-8-(4-methylpiperazin-1-yl)-N-(4-(4-propanoylpiperazin-1-yl)phenyl)chroman-2-carboxamide; and
(+)-N-(4-(4-ethanoylpiperazin-1-yl)phenyl)-6-methoxy-8-(4-methylpiperazin-1-yl)chroman-2-carboxamide;
or a pharmaceutically-acceptable salt thereof.

31. A method of treating mood disorders in a mammal comprising administering to such mammal an effective amount of a compound of formula I 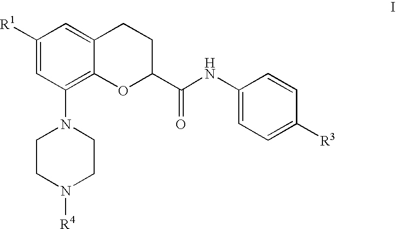
wherein:
R¹ is F or OCH₃;
R⁴ is CH₃, or C₂₋₄alkyl; and
R³ is selected from morpholine or piperazine attached by nitrogen and optionally substituted on the other nitrogen (for piperazine) with —C(═O)CH₃, —CH₃, —C(═O)CH₂CH₃, —C(═O)N(CH₃)₂, —SO₂CH₂CH₃, or —SO₂CH₃,
with the proviso that when R¹ is F and R⁴ is methyl then R³ cannot be morpholine; and
with the proviso that when R¹ is F and R⁴ is methyl then R³ cannot be piperazine substituted with —CH₂OH, —CH₂CH₂OH, —SO₂CH₃, or —SO₂CH₂CH₃;
or a pharmaceutically-acceptable salt thereof.

32. The method of claim 31 wherein the compound is selected from:
6-methoxy-8-(4-methylpiperazin-1-yl)-N-(4-morpholinophenyl)chroman-2-carboxamide;
(+)-6-methoxy-8-(4-methylpiperazin-1-yl)-N-(4-morpholinophenyl)chroman-2-carboxamide;
(−)-6-methoxy-8-(4-methylpiperazin-1-yl)-N-(4-morpholinophenyl)chroman-2-carboxamide;
N-(4-(4-ethanoylpiperazin-1-yl)phenyl)-6-fluoro-8-(4-methylpiperazin-1-yl)chroman-2-carboxamide;
(+)-N-(4-(4-ethanoylpiperazin-1-yl)phenyl)-6-fluoro-8-(4-methylpiperazin-1-yl)chroman-2-carboxamide;
(−)-N-(4-(4-ethanoylpiperazin-1-yl)phenyl)-6-fluoro-8-(4-methylpiperazin-1-yl)chroman-2-carboxamide;
N-(4-(4-ethanoylpiperazin-1-yl)phenyl)-6-fluoro-8-(4-methylpiperazin-1-yl)chroman-2-carboxamide;
(+)-N-(4-(4-ethanoylpiperazin-1-yl)phenyl)-6-fluoro-8-(4-methylpiperazin-1-yl)chroman-2-carboxamide;
(−)-N-(4-(4-ethanoylpiperazin-1-yl)phenyl)-6-fluoro-8-(4-methylpiperazin-1-yl)chroman-2-carboxamide;
6-fluoro-8-(4-methylpiperazin-1-yl)-N-(4-(4-propanoylpiperazin-1-yl)phenyl)chroman-2-carboxamide;
(+)-6-fluoro-8-(4-methylpiperazin-1-yl)-N-(4-(4-propanoylpiperazin-1-yl)phenyl)chroman-2- carboxamide;
(−)-6-fluoro-8-(4-methylpiperazin-1-yl)-N-(4-(4-propanoylpiperazin-1-yl)phenyl)chroman-2-carboxamide;
6-methoxy-8-(4-methylpiperazin-1-yl)-N-(4-(4-methylpiperazin-1-yl)phenyl)chroman-2-carboxamide;
(+)-6-methoxy-8-(4-methylpiperazin-1-yl)-N-(4-(4-methylpiperazin-1-yl)phenyl)chroman-2-carboxamide;
(−)-6-methoxy-8-(4-methylpiperazin-1-yl)-N-(4-(4-methylpiperazin-1-yl)phenyl)chroman-2-carboxamide;
6-fluoro-8-(4-methylpiperazin-1-yl)-N-(4-(4-methylpiperazin-1-yl)phenyl)chroman-2-carboxamide;
(+)-6-fluoro-8-(4-methylpiperazin-1-yl)-N-(4-(4-methylpiperazin-1-yl)phenyl)chroman-2-carboxamide;
(−)-6-fluoro-8-(4-methylpiperazin-1-yl)-N-(4-(4-methylpiperazin-1-yl)phenyl)chroman-2-carboxamide;
6-fluoro-8-(4-methylpiperazin-1-yl)-N-(4-(4-propanoylpiperazin-1-yl)phenyl)chroman-2-carboxamide;
(+)-6-fluoro-8-(4-methylpiperazin-1-yl)-N-(4-(4-propanoylpiperazin-1-yl)phenyl)chroman-2-carboxamide;
(−)-6-fluoro-8-(4-methylpiperazin-1-yl)-N-(4-(4-propanoylpiperazin-1-yl)phenyl)chroman-2-carboxamide;
N-(4-(4-(ethylsulfonyl)piperazin-1-yl)phenyl)-6-methoxy-8-(4-methylpiperazin-1-yl)chroman-2-carboxamide;
(+)-N-(4-(4-(ethylsulfonyl)piperazin-1-yl)phenyl)-6-methoxy-8-(4-methylpiperazin-1-yl)chroman-2-carboxamide;
(−)-N-(4-(4-(ethylsulfonyl)piperazin-1-yl)phenyl)-6-methoxy-8-(4-methylpiperazin-1-yl)chroman-2-carboxamide;
8-(4-butylpiperazin-1-yl)-6-methoxy-N-(4-morpholinophenyl)chroman-2-carboxamide;
(+)-8-(4-butylpiperazin-1-yl)-6-methoxy-N-(4-morpholinophenyl)chroman-2-carboxamide;
(−)-8-(4-butylpiperazin-1-yl)-6-methoxy-N-(4-morpholinophenyl)chroman-2-carboxamide;
6-methoxy-N-(4-morpholinophenyl)-8-(4-propylpiperazin-1-yl)chroman-2-carboxamide;
(+)-6-methoxy-N-(4-morpholinophenyl)-8-(4-propylpiperazin-1-yl)chroman-2-carboxamide;
(−)-6-methoxy-N-(4-morpholinophenyl)-8-(4-propylpiperazin-1-yl)chroman-2-carboxamide;
8-(4-isopropylpiperazin-1-yl)-6-methoxy-N-(4-morpholinophenyl)chroman-2-carboxamide;
(+)-8-(4-isopropylpiperazin-1-yl)-6-methoxy-N-(4-morpholinophenyl)chroman-2-carboxamide;
(−)-8-(4-isopropylpiperazin-1-yl)-6-methoxy-N-(4-morpholinophenyl)chroman-2-carboxamide;
8-(4-ethylpiperazin-1-yl)-6-methoxy-N-(4-morpholinophenyl)chroman-2-carboxamide;
(+)-8-(4-ethylpiperazin-1-yl)-6-methoxy-N-(4-morpholinophenyl)chroman-2-carboxamide;
(−)-8-(4-ethylpiperazin-1-yl)-6-methoxy-N-(4-morpholinophenyl)chroman-2-carboxamide;
6-methoxy-8-(4-methylpiperazin-1-yl)-N-(4-(4-propanoylpiperazin-1-yl)phenyl)chroman-2-carboxamide;
(+)-6-methoxy-8-(4-methylpiperazin-1-yl)-N-(4-(4-propanoylpiperazin-1-yl)phenyl)chroman-2-carboxamide;
(−)-6-methoxy-8-(4-methylpiperazin-1-yl)-N-(4-(4-propanoylpiperazin-1-yl)phenyl)chroman-2-carboxamide;
4-(4-(6-methoxy-8-(4-methylpiperazin-1-yl)chroman-2-carboxamido)phenyl)-N,N-dimethylpiperazine-1-carboxamide;
(+)-4-(4-(6-methoxy-8-(4-methylpiperazin-1-yl)chroman-2-carboxamido)phenyl)-N,N-dimethylpiperazine-1-carboxamide;
(−)-4-(4-(6-methoxy-8-(4-methylpiperazin-1-yl)chroman-2-carboxamido)phenyl)-N,N-dimethylpiperazine-1-carboxamide;
4-(4-(6-methoxy-8-(4-methylpiperazin-1-yl)chroman-2-carboxamido)phenyl)-N,N-dimethylpiperazine-1-carboxamide;
(+)-4-(4-(6-methoxy-8-(4-methylpiperazin-1-yl)chroman-2-carboxamido)phenyl)-N,N-dimethylpiperazine-1-carboxamide;
(−)-4-(4-(6-methoxy-8-(4-methylpiperazin-1-yl)chroman-2-carboxamido)phenyl)-N,N-dimethylpiperazine-1-carboxamide;
4-(4-(6-fluoro-8-(4-methylpiperazin-1-yl )chroman-2-carboxamido)phenyl)-N,N-dimethylpiperazine-1-carboxamide;
(+)-4-(4-(6-fluoro-8-(4-methylpiperazin-1-yl)chroman-2-carboxamido)phenyl)-N,N-dimethylpiperazine-1-carboxamide;
(−)-4-(4-(6-fluoro-8-(4-methylpiperazin-1-yl)chroman-2-carboxamido)phenyl)-N,N-dimethylpiperazine-1-carboxamide;
6-methoxy-8-(4-methylpiperazin-1-yl)-N-(4-(4-(methylsulfonyl)piperazin-1-yl)phenyl)chroman-2-carboxamide;
(+)-6-methoxy-8-(4-methylpiperazin-1-yl)-N-(4-(4-(methylsulfonyl)piperazin-1-yl)phenyl)chroman-2-carboxamide;
(−)-6-methoxy-8-(4-methylpiperazin-1-yl)-N-(4-(4-(methylsulfonyl)piperazin-1-yl)phenyl)chroman-2-carboxamide;
N-(4-(4-ethanoylpiperazin-1-yl)phenyl)-6-methoxy-8-(4-methylpiperazin-1-yl)chroman-2-carboxamide;
(+)-N-(4-(4-ethanoylpiperazin-1-yl)phenyl)-6-methoxy-8-(4-methylpiperazin-1-yl)chroman-2-carboxamide;
(−)-N-(4-(4-ethanoylpiperazin-1-yl)phenyl)-6-methoxy-8-(4-methylpiperazin-1-yl)chroman-2-carboxamide;
6-methoxy-8-(4-methylpiperazin-1-yl)-N-(4-(4-propanoylpiperazin-1-yl)phenyl)chroman-2-carboxamide;
(+) 6-methoxy-8-(4-methylpiperazin-1-yl)-N-(4-(4-propanoylpiperazin-1-yl)phenyl)chroman-2-carboxamide;
(−)-6-methoxy-8-(4-methylpiperazin-1-yl)-N-(4-(4-propanoylpiperazin-1-yl)phenyl)chroman-2-carboxamide;
N-(4-(4-ethanoylpiperazin-1-yl)phenyl)-6-methoxy-8-(4-methylpiperazin-1-yl)chroman-2-carboxamide;
(+)-N-(4-(4-ethanoylpiperazin-1-yl)phenyl)-6-methoxy-8-(4-methylpiperazin-1-yl)chroman-2-carboxamide; and
(−)-N-(4-(4-ethanoylpiperazin-1-yl)phenyl)-6-methoxy-8-(4-methylpiperazin-1-yl)chroman-2-carboxamide;
or a pharmaceutically-acceptable salt thereof.

33. The method of claim 31 wherein the compound is selected from:
(+)-6-methoxy-8-(4-methylpiperazin-1-yl)-N-(4-morpholinophenyl)chroman-2-carboxamide;
(+)-N-(4-(4-ethanoylpiperazin-1-yl)phenyl)-6-fluoro-8-(4-methylpiperazin-1-yl)chroman-2-carboxamide;
(+)-N-(4-(4-ethanoylpiperazin-1-yl)phenyl)-6-fluoro-8-(4-methylpiperazin-1-yl)chroman-2-carboxamide;
(+)-6-fluoro-8-(4-methylpiperazin-1-yl)-N-(4-(4-propanoylpiperazin-1-yl)phenyl)chroman-2-carboxamide;
(+)-6-methoxy-8-(4-methylpiperazin-1-yl)-N-(4-(4-methylpiperazin-1-yl)phenyl)chroman-2-carboxamide;
(+)-6-fluoro-8-(4-methylpiperazin-1-yl)-N-(4-(4-methylpiperazin-1-yl)phenyl)chroman-2-carboxamide;
(+)-6-fluoro-8-(4-methylpiperazin-1-yl)-N-(4-(4-propanoylpiperazin-1-yl)phenyl)chroman-2-carboxamide;
(+)-N-(4-(4-(ethylsulfonyl)piperazin-1-yl)phenyl)-6-methoxy-8-(4-methylpiperazin-1-yl)chroman-2-carboxamide
(+)-8-(4-butylpiperazin-1-yl)-6-methoxy-N-(4-morpholinophenyl)chroman-2-carboxamide;
(+)-6-methoxy-N-(4-morpholinophenyl)-8-(4-propylpiperazin-1-yl)chroman-2-carboxamide;
(+)-8-(4-isopropylpiperazin-1-yl)-6-methoxy-N-(4-morpholinophenyl)chroman-2-carboxamide;
(+)-8-(4-ethylpiperazin-1-yl)-6-methoxy-N-(4-morpholinophenyl)chroman-2-carboxamide;
(+)-6-methoxy-8-(4-methylpiperazin-1-yl)-N-(4-(4-propanoylpiperazin-1-yl)phenyl)chroman-2-carboxamide;
(+)-4-(4-(6-methoxy-8-(4-methylpiperazin-1-yl)chroman-2-carboxamido)phenyl)-N,N-dimethylpiperazine-1-carboxamide;
(+)-4-(4-(6-methoxy-8-(4-methylpiperazin-1-yl)chroman-2-carboxamido)phenyl)-N,N-dimethylpiperazine-1-carboxamide;
(+)-4-(4-(6-fluoro-8-(4-methylpiperazin-1-yl)chroman-2-carboxamido)phenyl)-N,N-dimethylpiperazine-1-carboxamide;
(+)-6-methoxy-8-(4-methylpiperazin-1-yl)-N-(4-(4-(methylsulfonyl)piperazin-1-yl)phenyl)chroman-2-carboxamide;
(+)-N-(4-(4-ethanoylpiperazin-1-yl)phenyl)-6-methoxy-8-(4-methylpiperazin-1-yl)chroman-2-carboxamide;
(+) 6-methoxy-8-(4-methylpiperazin-1-yl)-N-(4-(4-propanoylpiperazin-1-yl)phenyl)chroman-2-carboxamide; and
(+)-N-(4-(4-ethanoylpiperazin-1-yl)phenyl)-6-methoxy-8-(4-methylpiperazin-1-yl)chroman-2-carboxamide;
or a pharmaceutically-acceptable salt thereof.

34. A method of treating cognitive disorders in a mammal comprising administering to such mammal an effective amount of a compound of formula I 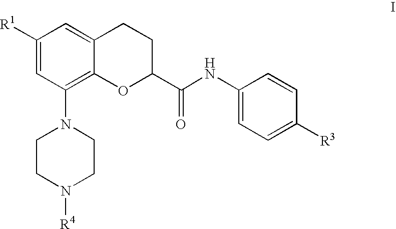
wherein:
R¹ is For OCH₃;
R⁴ is CH₃, or C₂₋₄alkyl; and
R³ is selected from morpholine or piperazine attached by nitrogen and optionally substituted on the other nitrogen (for piperazine) with —C(═O)CH₃, —CH₃, —C(═O)CH₂CH₃, —C(═O)N(CH₃)₂, —SO₂CH₂CH₃, or —SO₂CH₃,
with the proviso that when R¹ is F and R⁴ is methyl then R³ cannot be morpholine; and
with the proviso that when R¹ is F and R⁴is methyl then R³ cannot be piperazine substituted with —CH₂OH, —CH₂CH₂OH, —SO₂CH₃, or —SO₂CH₂CH₃;
or a pharmaceutically-acceptable salt thereof.

35. The method of claim 34 wherein the compound is selected from:
6-methoxy-8-(4-methylpiperazin-1-yl)-N-(4-morpholinophenyl)chroman-2-carboxamide;
(+)-6-methoxy-8-(4-methylpiperazin-1-yl)-N-(4-morpholinophenyl)chroman-2-carboxamide;
(−)-6-methoxy-8-(4-methylpiperazin-1-yl)-N-(4-morpholinophenyl)chroman-2-carboxamide;
N-(4-(4-ethanoylpiperazin-1-yl)phenyl)-6-fluoro-8-(4-methylpiperazin-1-yl)chroman-2-carboxamide;
(+)-N-(4-(4-ethanoylpiperazin-1-yl)phenyl)-6-fluoro-8-(4-methylpiperazin-1-yl)chroman-2-carboxamide;
(−)-N-(4-(4-ethanoylpiperazin-1-yl)phenyl)-6-fluoro-8-(4-methylpiperazin-1-yl)chroman-2-carboxamide;
N-(4-(4-ethanoylpiperazin-1-yl)phenyl)-6-fluoro-8-(4-methylpiperazin-1-yl)chroman-2-carboxamide;
(+)-N-(4-(4-ethanoylpiperazin-1-yl)phenyl)-6-fluoro-8-(4-methylpiperazin-1-yl)chroman-2-carboxamide;
(−)-N-(4-(4-ethanoylpiperazin-1-yl)phenyl)-6-fluoro-8-(4-methylpiperazin-1-yl)chroman-2-carboxamide;
6-fluoro-8-(4-methylpiperazin-1-yl)-N-(4-(4-propanoylpiperazin-1-yl)phenyl)chroman-2-carboxamide;
(+)-6-fluoro-8-(4-methylpiperazin-1-yl)-N-(4-(4-propanoylpiperazin-1-yl)phenyl)chroman-2-carboxamide;
(−)-6-fluoro-8-(4-methylpiperazin-1-yl)-N-(4-(4-propanoylpiperazin-1-yl)phenyl)chroman-2-carboxamide;
6-methoxy-8-(4-methylpiperazin-1-yl)-N-(4-(4-methylpiperazin-1-yl)phenyl)chroman-2-carboxamide;
(+)-6-methoxy-8-(4-methylpiperazin-1-yl)-N-(4-(4-methylpiperazin-1-yl)phenyl)chroman-2-carboxamide;
(−)-6-methoxy-8-(4-methylpiperazin-1-yl)-N-(4-(4-methylpiperazin-1-yl)phenyl)chroman-2-carboxamide;
6-fluoro-8-(4-methylpiperazin-1-yl)-N-(4-(4-methylpiperazin-1-yl)phenyl)chroman-2-carboxamide;
(+)-6-fluoro-8-(4-methylpiperazin-1-yl)-N-(4-(4-methylpiperazin-1-yl)phenyl)chroman-2-carboxamide;
(−)-6-fluoro-8-(4-methylpiperazin-1-yl)-N-(4-(4-methylpiperazin-1-yl)phenyl)chroman-2-carboxamide;
6-fluoro-8-(4-methylpiperazin-1-yl)-N-(4-(4-propanoylpiperazin-1-yl)phenyl)chroman-2-carboxamide;
(+)-6-fluoro-8-(4-methylpiperazin-1-yl)-N-(4-(4-propanoylpiperazin-1-yl)phenyl)chroman-2-carboxamide;
(−)-6-fluoro-8-(4-methylpiperazin-1-yl)-N-(4-(4-propanoylpiperazin-1-yl)phenyl)chroman-2-carboxamide;
N-(4-(4-(ethylsulfonyl)piperazin-1-yl)phenyl)-6-methoxy-8-(4-methylpiperazin-1-yl)chroman-2-carboxamide;
(+)-N-(4-(4-(ethylsulfonyl)piperazin-1-yl)phenyl)-6-methoxy-8-(4-methylpiperazin-1-yl)chroman-2-carboxamide;
(−)-N-(4-(4-(ethylsulfonyl)piperazin-1-yl)phenyl)-6-methoxy-8-(4-methylpiperazin-1-yl)chroman-2-carboxamide;
8-(4-butylpiperazin-1-yl)-6-methoxy-N-(4-morpholinophenyl)chroman-2-carboxamide;
(+)-8-(4-butylpiperazin-1-yl)-6-methoxy-N-(4-morpholinophenyl)chroman-2-carboxamide;
(−)-8-(4-butylpiperazin-1-yl)-6-methoxy-N-(4-morpholinophenyl)chroman-2-carboxamide;
6-methoxy-N-(4-morpholinophenyl)-8-(4-propylpiperazin-1-yl)chroman-2-carboxamide;
(+)-6-methoxy-N-(4-morpholinophenyl)-8-(4-propylpiperazin-1-yl)chroman-2-carboxamide;
(−)-6-methoxy-N-(4-morpholinophenyl)-8-(4-propylpiperazin-1-yl)chroman-2-carboxamide;
8-(4-isopropylpiperazin-1-yl)-6-methoxy-N-(4-morpholinophenyl)chroman-2-carboxamide;
(+)-8-(4-isopropylpiperazin-1-yl)-6-methoxy-N-(4-morpholinophenyl)chroman-2-carboxamide;
(−)-8-(4-isopropylpiperazin-1-yl)-6-methoxy-N-(4-morpholinophenyl)chroman-2-carboxamide;
8-(4-ethylpiperazin-1-yl)-6-methoxy-N-(4-morpholinophenyl)chroman-2-carboxamide;
(+)-8-(4-ethylpiperazin-1-yl)-6-methoxy-N-(4-morpholinophenyl)chroman-2-carboxamide;
(−)-8-(4-ethylpiperazin-1-yl)-6-methoxy-N-(4-morpholinophenyl)chroman-2-carboxamide;
6-methoxy-8-(4-methylpiperazin-1-yl)-N-(4-(4-propanoylpiperazin-1-yl)phenyl)chroman-2-carboxamide;
(+)-6-methoxy-8-(4-methylpiperazin-1-yl)-N-(4-(4-propanoylpiperazin-1-yl)phenyl)chroman-2-carboxamide;
(−)-6-methoxy-8-(4-methylpiperazin-1-yl)-N-(4-(4-propanoylpiperazin-1-yl)phenyl)chroman-2-carboxamide;
4-(4-(6-methoxy-8-(4-methylpiperazin-1-yl)chroman-2-carboxamido)phenyl)-N,N-dimethylpiperazine-1-carboxamide;
(+)-4-(4-(6-methoxy-8-(4-methylpiperazin-1-yl)chroman-2-carboxamido)phenyl)-N,N-dimethylpiperazine-1-carboxamide;
(−)-4-(4-(6-methoxy-8-(4-methylpiperazin-1-yl)chroman-2-carboxamido)phenyl)-N,N-dimethylpiperazine-1-carboxamide;
4-(4-(6-methoxy-8-(4-methylpiperazin-1-yl)chroman-2-carboxamido)phenyl)-N,N-dimethylpiperazine-1-carboxamide;
(+)-4-(4-(6-methoxy-8-(4-methylpiperazin-1-yl)chroman-2-carboxamido)phenyl)-N,N-dimethylpiperazine-1-carboxamide;
(−)-4-(4-(6-methoxy-8-(4-methylpiperazin-1-yl)chroman-2-carboxamido)phenyl)-N,N-dimethylpiperazine-1-carboxamide;
4-(4-(6-fluoro-8-(4-methylpiperazin-1-yl)chroman-2-carboxamido)phenyl)-N,N-dimethylpiperazine-1-carboxamide;
(+)-4-(4-(6-fluoro-8-(4-methylpiperazin-1-yl)chroman-2-carboxamido)phenyl)-N,N-dimethylpiperazine-1-carboxamide;
(−)-4-(4-(6-fluoro-8-(4-methylpiperazin-1-yl)chroman-2-carboxamido)phenyl)-N,N-dimethylpiperazine-1-carboxamide;
6-methoxy-8-(4-methylpiperazin-1-yl)-N-(4-(4-(methylsulfonyl)piperazin-1-yl)phenyl)chroman-2-carboxamide;
(+)-6-methoxy-8-(4-methylpiperazin-1-yl)-N-(4-(4-(methylsulfonyl)piperazin-1-yl)phenyl)chroman-2-carboxamide;
(−)-6-methoxy-8-(4-methylpiperazin-1-yl)-N-(4-(4-(methylsulfonyl)piperazin-1-yl)phenyl)chroman-2-carboxamide;
N-(4-(4-ethanoylpiperazin-1-yl)phenyl)-6-methoxy-8-(4-methylpiperazin-1-yl)chroman-2-carboxamide;
(+)-N-(4-(4-ethanoylpiperazin-1-yl)phenyl)-6-methoxy-8-(4-methylpiperazin-1-yl)chroman-2-carboxamide;
(−)-N-(4-(4-ethanoylpiperazin-1-yl)phenyl)-6-methoxy-8-(4-methylpiperazin-1-yl)chroman-2-carboxamide;
6-methoxy-8-(4-methylpiperazin-1-yl)-N-(4-(4-propanoylpiperazin-1-yl)phenyl)chroman-2-carboxamide;
(+) 6-methoxy-8-(4-methylpiperazin-1-yl)-N-(4-(4-propanoylpiperazin-1-yl)phenyl)chroman-2-carboxamide;
(−)-6-methoxy-8-(4-methylpiperazin-1-yl)-N-(4-(4-propanoylpiperazin-1-yl)phenyl)chroman-2-carboxamide;
N-(4-(4-ethanoylpiperazin-1-yl)phenyl)-6-methoxy-8-(4-methylpiperazin-1-yl)chroman-2-carboxamide;
(+)-N-(4-(4-ethanoylpiperazin-1-yl)phenyl)-6-methoxy-8-(4-methylpiperazin-1-yl)chroman-2-carboxamide; and
(−)-N-(4-(4-ethanoylpiperazin-1-yl)phenyl)-6-methoxy-8-(4-methylpiperazin-1-yl)chroman-2-carboxamide;
or a pharmaceutically-acceptable salt thereof.

36. The method of claim 34 wherein the compound is selected from:
(+)-6-methoxy-8-(4-methylpiperazin-1-yl)-N-(4-morpholinophenyl)chroman-2-carboxamide;
(+)-N-(4-(4-ethanoylpiperazin-1-yl)phenyl)-6-fluoro-8-(4-methylpiperazin-1-yl)chroman-2-carboxamide;
(+)-N-(4-(4-ethanoylpiperazin-1-yl)phenyl)-6-fluoro-8-(4-methylpiperazin-1-yl)chroman-2-carboxamide;
(+)-6-fluoro-8-(4-methylpiperazin-1-yl)-N-(4-(4-propanoylpiperazin-1-yl)phenyl)chroman-2-carboxamide;
(+)-6-methoxy-8-(4-methylpiperazin-1-yl)-N-(4-(4-methylpiperazin-1-yl)phenyl)chroman-2-carboxamide;
(+)-6-fluoro-8-(4-methylpiperazin-1-yl)-N-(4-(4-methylpiperazin-1-yl)phenyl)chroman-2-carboxamide;
(+)-6-fluoro-8-(4-methylpiperazin-1-yl)-N-(4-(4-propanoylpiperazin-1-yl)phenyl)chroman-2-carboxamide;
(+)-N-(4-(4-(ethylsulfonyl)piperazin-1-yl)phenyl)-6-methoxy-8-(4-methylpiperazin-1-yl)chroman-2-carboxamide
(+)-8-(4-butylpiperazin-1-yl)-6-methoxy-N-(4-morpholinophenyl)chroman-2-carboxamide;
(+)-6-methoxy-N-(4-morpholinophenyl)-8-(4-propylpiperazin-1-yl)chroman-2-carboxamide;
(+)-8-(4-isopropylpiperazin-1-yl)-6-methoxy-N-(4-morpholinophenyl)chroman-2-carboxamide;
(+)-8-(4-ethylpiperazin-1-yl)-6-methoxy-N-(4-morpholinophenyl)chroman-2-carboxamide;
(+)-6-methoxy-8-(4-methylpiperazin-1-yl)-N-(4-(4-propanoylpiperazin-1-yl)phenyl)chroman-2-carboxamide;
(+)-4-(4-(6-methoxy-8-(4-methylpiperazin-1-yl)chroman-2-carboxamido)phenyl)-N,N-dimethylpiperazine-1-carboxamide;
(+)-4-(4-(6-methoxy-8-(4-methylpiperazin-1-yl)chroman-2-carboxamido)phenyl)-N,N-dimethylpiperazine-1-carboxamide;
(+)-4-(4-(6-fluoro-8-(4-methylpiperazin-1-yl)chroman-2-carboxamido)phenyl)-N,N-dimethylpiperazine-1-carboxamide;
(+)-6-methoxy-8-(4-methylpiperazin-1-yl)-N-(4-(4-(methylsulfonyl)piperazin-1-yl)phenyl)chroman-2-carboxamide;
(+)-N-(4-(4-ethanoylpiperazin-1-yl)phenyl)-6-methoxy-8-(4-methylpiperazin-1-yl)chroman-2-carboxamide;
(+) 6-methoxy-8-(4-methylpiperazin-1-yl)-N-(4-(4-propanoylpiperazin-1-yl)phenyl)chroman-2-carboxamide; and
(+)-N-(4-(4-ethanoylpiperazin-1-yl)phenyl)-6-methoxy-8-(4-methylpiperazin-1-yl)chroman-2-carboxamide;
or a pharmaceutically-acceptable salt thereof.

37. The use of a compound according to any one of claims 1 to 27 in the preparation of a medicament for the treatment of anxiety disorders or mood disorders or cognitive disorders.

38. A pharmaceutical composition comprising a-compound according to any one of claims 1 to 27 or a pharmaceutically acceptable salt thereof and a pharmaceutically acceptable carrier.

39. A process for the preparation of a compound according to claim 1 or a pharmaceutically acceptable salt thereof, which process comprises reacting a compound of formula (II): 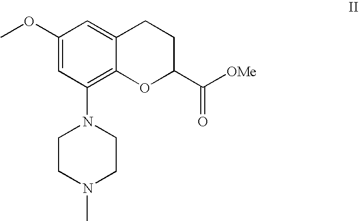
with lithium hydroxide to yield a compound of formula (III): 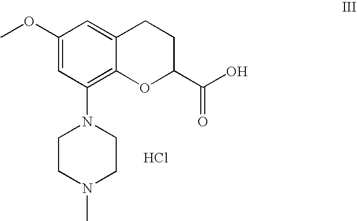
to which can be added an appropriate aniline to achieve the desired compound as shown in formula IV. [chemical structure]
